SEQUENCE LISTING (73 sequences)

SEQ ID NO: 1 - protein, 177 aa, Homo sapiens
MFHVSFRYIFGLPPLILVLLPVASSDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEH

SEQ ID NO: 2 - protein, 154 aa, Rattus norvegicus
MFHVSFRYIFGIPPLILVLLPVTSSDCHIKDKDGKAFGSVLMISINQLDKMTGTDSDCPNNEPNFFKKHLCDDTKEAAFLNRAARKLRQFLKMNISEEFNDHLLRVSDGTQTLVNCTSKEEKTIKEQKKNDPCFLKRLLREIKTCWNKILKGSI

SEQ ID NO: 3 - protein, 154 aa, Mus Musculus
MFHVSFRYIFGIPPLILVLLPVTSSECHIKDKEGKAYESVLMISIDELDKMTGTDSNCPNNEPNFFRKHVCDDTKEAAFLNRAARKLKQFLKMNISEEFNVHLLTVSQGTQTLVNCTSKEEKNVKEQKKNDACFLKRLLREIKTCWNKILKGSI

SEQ ID NO: 4 - protein, 177 aa, Cercopithecus aethiops
MFHVSFRYIFGLPPLILVLLPVASSDCDIEGKDGKQYESVLMVSIDQLLDSMKEIGSNCLNNEFNFFKRHLCDDNKEGMFLFRAARKLKQFLKMNSTGDFDLHLLKVSEGTTILLNCTGKVKGRKPAALGEPQPTKSLEENKSLKEQKKLNDSCFLKRLLQKIKTCWNKILMGTKEH

SEQ ID NO: 5 - protein, 176 aa, Bos taurus
MFHVSFRYIFGIPPLILVLLPVASSDCDISGKDGGAYQNVLMVNIDDLDNMINFDSNCLNNEPNFFKKHSCDDNKEASFLNRASRKLRQFLKMNISDDFKLHLSTVSQGTLTLLNCTSKGKGRKPPSLSEAQPTKNLEENKSSKEQKKQNDLCFLKILLQKIKTCWNKILRGIKEH

SEQ ID NO: 6 - protein, 176 aa, Ovis aries
MFHVSFRYIFGIPPLILVLLPVASSDCDFSGKDGGAYQNVLMVSIDDLDNMINFDSNCLNNEPNFFKKHSCDDNKEASFLNRAARKLKQFLKMNISDDFKLHLSTVSQGTLTLLNCTSKGKGRKPPSLGEAQPTKNLEENKSLKEQRKQNDLCFLKILLQKIKTCWNKILRGITEH

SEQ ID NO: 7 - protein, 384 aa, Artificial Sequence
  amino acid sequence of human IgD constant region (Genbank accession No. P01880)
APTKAPDVFPIISGCRHPKDNSPVVLACLITGYHPTSVTVTWYMGTQSQPQRTFPEIQRRDSYYMTSSQLSTPLQQWRQGEYKCVVQHTASKSKKEIFRWPESPKAQASSVPTAQPQAEGSLAKATTAPATTRNTGRGGEEKKKEKEKEEQEERETKTPECPSHTQPLGVYLLTPAVQDLWLRDKATFTCFVVGSDLKDAHLTWEVAGKVPTGGVEEGLLERHSNGSQSQHSRLTLPRSLWNAGTSVTCTLNHPSLPPQRLMALREPAAQAPVKLSLNLLASSDPPEAASWLLCEVSGFSPPNILLMWLEDQREVNTSGFAPARPPPQPGSTTFWAWSVLRVPAPPSPQPATYTCVVSHEDSRTLLNASRSLEVSYVTDHGPMK

SEQ ID NO: 8 - protein, 327 aa, Artificial Sequence
  amino acid sequence of Partial human IgG4 constant region (Genbank accession No. AAH25985)
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 9 - protein, 245 aa, Artificial Sequence
  amino acid sequence of hyFc
RNTGRGGEEKKKEKEKEEQEERETKTPECPSHTQPLGVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 10 - protein, 245 aa, Artificial Sequence
  amino acid sequence of hyFcM1
RNTGRGGEEKKGGKEKEEQEERETKTPECPSHTQPLGVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 11 - protein, 245 aa, Artificial Sequence
  amino acid sequence of hyFcM2
RNTGRGGEEKKGSKEKEEQEERETKTPECPSHTQPLGVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 12 - protein, 245 aa, Artificial Sequence
  amino acid sequence of hyFcM3
RNTGRGGEEKKSGKEKEEQEERETKTPECPSHTQPLGVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 13 - protein, 245 aa, Artificial Sequence
  amino acid sequence of hyFcM4
RNTGRGGEEKKSSKEKEEQEERETKTPECPSHTQPLGVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 14 - protein, 243 aa, Artificial Sequence
  amino acid sequence of mouse IgG Fc variant
ASAEPRGPTIKPCPPCKCPAPNLEGGPSVFIFPPKIKDVLMISLSPIVTCVVVDVSEDDPDVQISWFVNNVEVHTAQTQTHREDYNSTLRVVSALPIQHQDWMSGKAFACAVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGKGGGNSGS

SEQ ID NO: 15 - protein, 153 aa, Artificial Sequence
  amino acid sequence of modified IL-7(M)
MDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEH

SEQ ID NO: 16 - protein, 154 aa, Artificial Sequence
  amino acid sequence of modified IL-7(MM)
MMDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEH

SEQ ID NO: 17 - protein, 155 aa, Artificial Sequence
  amino acid sequence of modified IL-7(MMM)
MMMDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEH

SEQ ID NO: 18 - protein, 155 aa, Artificial Sequence
  amino acid sequence of modified IL-7(MGM)
MGMDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEH

SEQ ID NO: 19 - protein, 155 aa, Artificial Sequence
  amino acid sequence of modified IL-7(DDD)
DDDDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEH

SEQ ID NO: 20 - protein, 156 aa, Artificial Sequence
  amino acid sequence of modified IL-7(MMMM)
MMMMDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEH

SEQ ID NO: 21 - protein, 398 aa, Artificial Sequence
  amino acid sequence of modified IL-7(M) fused hyFc
MDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEHRNTGRGGEEKKKEKEKEEQEERETKTPECPSHTQPLGVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 22 - protein, 399 aa, Artificial Sequence
  amino acid sequence of modified IL-7(MM) fused hyFc
MMDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEHRNTGRGGEEKKKEKEKEEQEERETKTPECPSHTQPLGVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 23 - protein, 400 aa, Artificial Sequence
  amino acid sequence of modified IL-7(MMM) fused hyFc
MMMDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEHRNTGRGGEEKKKEKEKEEQEERETKTPECPSHTQPLGVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 24 - protein, 400 aa, Artificial Sequence
  amino acid sequence of modified IL-7(MGM) fused hyFc
MGMDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEHRNTGRGGEEKKKEKEKEEQEERETKTPECPSHTQPLGVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 25 - protein, 401 aa, Artificial Sequence
  amino acid sequence of modified IL-7(MMMM) fused hyFc
MMMMDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEHRNTGRGGEEKKKEKEKEEQEERETKTPECPSHTQPLGVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 26 - protein, 397 aa, Artificial Sequence
  amino acid sequence of human IL-7 fused hyFc
DCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEHRNTGRGGEEKKKEKEKEEQEERETKTPECPSHTQPLGVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 27 - protein, 395 aa, Artificial Sequence
  amino acid sequence of human IL-7 fused nonlytic mouse Fc
DCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEHASAEPRGPTIKPCPPCKCPAPNLEGGPSVFIFPPKIKDVLMISLSPIVTCVVVDVSEDDPDVQISWFVNNVEVHTAQTQTHREDYNSTLRVVSALPIQHQDWMSGKAFACAVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGKGGGNSGS

SEQ ID NO: 28 - DNA, 531 bp, Artificial Sequence
  nucleotide sequence of human IL-7
atgttccacgtgagcttcaggtacatcttcggcctgccacccctgatcctggtgctgctgcctgtggccagctccgactgcgacatcgagggaaaagacggcaagcagtacgaaagcgtgctgatggtgtccatcgaccagctgctggattctatgaaggagattgggagtaactgcctgaacaatgagttcaacttcttcaaacggcacatttgtgatgccaacaaggagggaatgttcctgtttcgggccgctagaaaactgaggcagttcctgaagatgaacagcaccggagactttgatctgcatctgctgaaagtgtctgagggcaccacaatcctgctgaactgcactgggcaggtgaaaggaaggaagcctgccgctctgggagaggctcagccaaccaagtcactggaggaaaacaaaagcctgaaggaacagaagaaactgaatgacctgtgctttctgaaacggctgctgcaggagatcaaaacatgttggaacaagattctgatgggcacaaaggaacac

SEQ ID NO: 29 - DNA, 534 bp, Artificial Sequence
  nucleotide sequence of modified IL-7(M)
atgttccacgtgagcttcagatacatcttcggcctgccccccctgatcctggtgctgctgcccgtggccagcagcatggactgcgacatcgagggcaaggacggcaagcagtacgagagcgtgctgatggtgagcatcgaccagctgctggacagcatgaaggagatcggcagcaactgcctgaacaacgagttcaacttcttcaagagacacatctgcgacgccaacaaggagggcatgttcctgttcagagccgccagaaagctgagacagttcctgaagatgaacagcaccggcgacttcgacctgcacctgctgaaggtgagcgagggcacaaccatcctgctgaactgcaccggccaggtgaagggcagaaagcccgccgccctgggcgaggcccagcccaccaagagcctggaggagaacaagagcctgaaggagcagaagaagctgaacgacctgtgcttcctgaagagactgctgcaggagatcaagacctgctggaacaagatcctgatgggcaccaaggagcac

SEQ ID NO: 30 - DNA, 537 bp, Artificial Sequence
  nucleotide sequence of modified IL-7(MM)
atgttccacgtgagcttcagatacatcttcggcctgccccccctgatcctggtgctgctgcccgtggccagcagcatgatggactgcgacatcgagggcaaggacggcaagcagtacgagagcgtgctgatggtgagcatcgaccagctgctggacagcatgaaggagatcggcagcaactgcctgaacaacgagttcaacttcttcaagagacacatctgcgacgccaacaaggagggcatgttcctgttcagagccgccagaaagctgagacagttcctgaagatgaacagcaccggcgacttcgacctgcacctgctgaaggtgagcgagggcacaaccatcctgctgaactgcaccggccaggtgaagggcagaaagcccgccgccctgggcgaggcccagcccaccaagagcctggaggagaacaagagcctgaaggagcagaagaagctgaacgacctgtgcttcctgaagagactgctgcaggagatcaagacctgctggaacaagatcctgatgggcaccaaggagcac

SEQ ID NO: 31 - DNA, 540 bp, Artificial Sequence
  nucleotide sequence of modified IL-7(MMM)
atgttccacgtgagcttcagatacatcttcggcctgccccccctgatcctggtgctgctgcccgtggccagcagcatgatgatggactgcgacatcgagggcaaggacggcaagcagtacgagagcgtgctgatggtgagcatcgaccagctgctggacagcatgaaggagatcggcagcaactgcctgaacaacgagttcaacttcttcaagagacacatctgcgacgccaacaaggagggcatgttcctgttcagagccgccagaaagctgagacagttcctgaagatgaacagcaccggcgacttcgacctgcacctgctgaaggtgagcgagggcacaaccatcctgctgaactgcaccggccaggtgaagggcagaaagcccgccgccctgggcgaggcccagcccaccaagagcctggaggagaacaagagcctgaaggagcagaagaagctgaacgacctgtgcttcctgaagagactgctgcaggagatcaagacctgctggaacaagatcctgatgggcaccaaggagcac

SEQ ID NO: 32 - DNA, 540 bp, Artificial Sequence
  nucleotide sequence of modified IL-7(MGM)
atgttccacgtgagcttcaggtacatcttcggcctgccacccctgatcctggtgctgctgcctgtggccagctccatggggatggactgcgacatcgagggaaaagacggcaagcagtacgaaagcgtgctgatggtgtccatcgaccagctgctggattctatgaaggagattgggagtaactgcctgaacaatgagttcaacttcttcaaacggcacatttgtgatgccaacaaggagggaatgttcctgtttcgggccgctagaaaactgaggcagttcctgaagatgaacagcaccggagactttgatctgcatctgctgaaagtgtctgagggcaccacaatcctgctgaactgcactgggcaggtgaaaggaaggaagcctgccgctctgggagaggctcagccaaccaagtcactggaggaaaacaaaagcctgaaggaacagaagaaactgaatgacctgtgctttctgaaacggctgctgcaggagatcaaaacatgttggaacaagattctgatgggcaccaaggagcac

SEQ ID NO: 33 - DNA, 540 bp, Artificial Sequence
  nucleotide sequence of modified IL-7(DDD)
atgttccacgtgagcttcagatacatcttcggcctgccccccctgatcctggtgctgctgcccgtggccagcagcgacgatgacgactgcgacatcgagggcaaggacggcaagcagtacgagagcgtgctgatggtgagcatcgaccagctgctggacagcatgaaggagatcggcagcaactgcctgaacaacgagttcaacttcttcaagagacacatctgcgacgccaacaaggagggcatgttcctgttcagagccgccagaaagctgagacagttcctgaagatgaacagcaccggcgacttcgacctgcacctgctgaaggtgagcgagggcacaaccatcctgctgaactgcaccggccaggtgaagggcagaaagcccgccgccctgggcgaggcccagcccaccaagagcctggaggagaacaagagcctgaaggagcagaagaagctgaacgacctgtgcttcctgaagagactgctgcaggagatcaagacctgctggaacaagatcctgatgggcaccaaggagcac

SEQ ID NO: 34 - DNA, 543 bp, Artificial Sequence
  nucleotide sequence of modified IL-7(MMMM)
atgttccacgtgagcttcagatacatcttcggcctgccccccctgatcctggtgctgctgcccgtggccagcagcatgatgatgatggactgcgacatcgagggcaaggacggcaagcagtacgagagcgtgctgatggtgagcatcgaccagctgctggacagcatgaaggagatcggcagcaactgcctgaacaacgagttcaacttcttcaagagacacatctgcgacgccaacaaggagggcatgttcctgttcagagccgccagaaagctgagacagttcctgaagatgaacagcaccggcgacttcgacctgcacctgctgaaggtgagcgagggcacaaccatcctgctgaactgcaccggccaggtgaagggcagaaagcccgccgccctgggcgaggcccagcccaccaagagcctggaggagaacaagagcctgaaggagcagaagaagctgaacgacctgtgcttcctgaagagactgctgcaggagatcaagacctgctggaacaagatcctgatgggcaccaaggagcac

SEQ ID NO: 35 - DNA, 1284 bp, Artificial Sequence
  nucleotide sequence of modified IL-7(M) fused hyFc
atgttccacgtgagcttcagatacatcttcggcctgccccccctgatcctggtgctgctgcccgtggccagcagcatggactgcgacatcgagggcaaggacggcaagcagtacgagagcgtgctgatggtgagcatcgaccagctgctggacagcatgaaggagatcggcagcaactgcctgaacaacgagttcaacttcttcaagagacacatctgcgacgccaacaaggagggcatgttcctgttcagagccgccagaaagctgagacagttcctgaagatgaacagcaccggcgacttcgacctgcacctgctgaaggtgagcgagggcacaaccatcctgctgaactgcaccggccaggtgaagggcagaaagcccgccgccctgggcgaggcccagcccaccaagagcctggaggagaacaagagcctgaaggagcagaagaagctgaacgacctgtgcttcctgaagagactgctgcaggagatcaagacctgctggaacaagatcctgatgggcaccaaggagcacaggaacacaggcagaggcggcgaggagaagaagaaggagaaggagaaggaggagcaggaggaaagagagaccaagacccccgagtgccccagccacacccagcccctgggcgtgttcctgttccctcccaagcccaaggacaccctgatgatcagcagaacccccgaggtgacctgcgtggtcgtggatgtgagccaggaagatcccgaagtgcagttcaactggtacgtggatggcgtggaagtgcacaacgccaagaccaagcccagagaagagcagttcaactccacctacagagtggtgagcgtgctgaccgtgctgcaccaggactggctgaacggcaaggagtacaagtgcaaggtgtccaacaaaggcctgcccagctccatcgagaagaccatcagcaaagccaaaggccagcccagagaaccccaggtgtacaccctgcctcccagccaggaagagatgaccaagaaccaggtgtccctgacctgcctggtgaaaggcttctaccccagcgacatcgccgtggagtgggaaagcaacggccagcccgagaacaattacaagacaacccctcccgtgctggatagcgatggcagcttctttctgtacagcagactgaccgtggacaagagcagatggcaggaaggcaacgtgttcagctgcagcgtgatgcacgaagccctgcacaaccactacacccagaagagcctgtccctgagcctgggcaagtgactcgagtctaga

SEQ ID NO: 36 - DNA, 1272 bp, Artificial Sequence
  nucleotide sequence of modified IL-7(MM) fused hyFc
atgttccacgtgagcttcagatacatcttcggcctgccccccctgatcctggtgctgctgcccgtggccagcagcatgatggactgcgacatcgagggcaaggacggcaagcagtacgagagcgtgctgatggtgagcatcgaccagctgctggacagcatgaaggagatcggcagcaactgcctgaacaacgagttcaacttcttcaagagacacatctgcgacgccaacaaggagggcatgttcctgttcagagccgccagaaagctgagacagttcctgaagatgaacagcaccggcgacttcgacctgcacctgctgaaggtgagcgagggcacaaccatcctgctgaactgcaccggccaggtgaagggcagaaagcccgccgccctgggcgaggcccagcccaccaagagcctggaggagaacaagagcctgaaggagcagaagaagctgaacgacctgtgcttcctgaagagactgctgcaggagatcaagacctgctggaacaagatcctgatgggcaccaaggagcacaggaacacaggcagaggcggcgaggagaagaagaaggagaaggagaaggaggagcaggaggaaagagagaccaagacccccgagtgccccagccacacccagcccctgggcgtgttcctgttccctcccaagcccaaggacaccctgatgatcagcagaacccccgaggtgacctgcgtggtcgtggatgtgagccaggaagatcccgaagtgcagttcaactggtacgtggatggcgtggaagtgcacaacgccaagaccaagcccagagaagagcagttcaactccacctacagagtggtgagcgtgctgaccgtgctgcaccaggactggctgaacggcaaggagtacaagtgcaaggtgtccaacaaaggcctgcccagctccatcgagaagaccatcagcaaagccaaaggccagcccagagaaccccaggtgtacaccctgcctcccagccaggaagagatgaccaagaaccaggtgtccctgacctgcctggtgaaaggcttctaccccagcgacatcgccgtggagtgggaaagcaacggccagcccgagaacaattacaagacaacccctcccgtgctggatagcgatggcagcttctttctgtacagcagactgaccgtggacaagagcagatggcaggaaggcaacgtgttcagctgcagcgtgatgcacgaagccctgcacaaccactacacccagaagagcctgtccctgagcctgggcaag

SEQ ID NO: 37 - DNA, 1275 bp, Artificial Sequence
  nucleotide sequence of modified IL-7(MMM) fused hyFc
atgttccacgtgagcttcagatacatcttcggcctgccccccctgatcctggtgctgctgcccgtggccagcagcatgatgatggactgcgacatcgagggcaaggacggcaagcagtacgagagcgtgctgatggtgagcatcgaccagctgctggacagcatgaaggagatcggcagcaactgcctgaacaacgagttcaacttcttcaagagacacatctgcgacgccaacaaggagggcatgttcctgttcagagccgccagaaagctgagacagttcctgaagatgaacagcaccggcgacttcgacctgcacctgctgaaggtgagcgagggcacaaccatcctgctgaactgcaccggccaggtgaagggcagaaagcccgccgccctgggcgaggcccagcccaccaagagcctggaggagaacaagagcctgaaggagcagaagaagctgaacgacctgtgcttcctgaagagactgctgcaggagatcaagacctgctggaacaagatcctgatgggcaccaaggagcacaggaacacaggcagaggcggcgaggagaagaagaaggagaaggagaaggaggagcaggaggaaagagagaccaagacccccgagtgccccagccacacccagcccctgggcgtgttcctgttccctcccaagcccaaggacaccctgatgatcagcagaacccccgaggtgacctgcgtggtcgtggatgtgagccaggaagatcccgaagtgcagttcaactggtacgtggatggcgtggaagtgcacaacgccaagaccaagcccagagaagagcagttcaactccacctacagagtggtgagcgtgctgaccgtgctgcaccaggactggctgaacggcaaggagtacaagtgcaaggtgtccaacaaaggcctgcccagctccatcgagaagaccatcagcaaagccaaaggccagcccagagaaccccaggtgtacaccctgcctcccagccaggaagagatgaccaagaaccaggtgtccctgacctgcctggtgaaaggcttctaccccagcgacatcgccgtggagtgggaaagcaacggccagcccgagaacaattacaagacaacccctcccgtgctggatagcgatggcagcttctttctgtacagcagactgaccgtggacaagagcagatggcaggaaggcaacgtgttcagctgcagcgtgatgcacgaagccctgcacaaccactacacccagaagagcctgtccctgagcctgggcaag

SEQ ID NO: 38 - DNA, 1275 bp, Artificial Sequence
  nucleotide sequence of modified IL-7(MGM) fused hyFc
atgttccacgtgagcttcaggtacatcttcggcctgccacccctgatcctggtgctgctgcctgtggccagctccatggggatggactgcgacatcgagggaaaagacggcaagcagtacgaaagcgtgctgatggtgtccatcgaccagctgctggattctatgaaggagattgggagtaactgcctgaacaatgagttcaacttcttcaaacggcacatttgtgatgccaacaaggagggaatgttcctgtttcgggccgctagaaaactgaggcagttcctgaagatgaacagcaccggagactttgatctgcatctgctgaaagtgtctgagggcaccacaatcctgctgaactgcactgggcaggtgaaaggaaggaagcctgccgctctgggagaggctcagccaaccaagtcactggaggaaaacaaaagcctgaaggaacagaagaaactgaatgacctgtgctttctgaaacggctgctgcaggagatcaaaacatgttggaacaagattctgatgggcacaaaggaacaccgcaatactgggcggggcggggaggaaaagaaaaaggagaaggaaaaggaggaacaggaggaaagagagactaagaccccagaatgtcccagccatactcagcccctgggggtgttcctgtttccccctaaacctaaggataccctgatgatcagcaggacacccgaggtgacctgcgtggtcgtggatgtgagccaggaagatcccgaagtgcagttcaactggtacgtggatggcgtggaagtgcacaacgccaagaccaagcccagagaagagcagttcaactccacctacagagtggtgagcgtgctgaccgtgctgcaccaggactggctgaacggcaaggagtacaagtgcaaggtgtccaacaaaggcctgcccagctccatcgagaagaccatcagcaaagccaaaggccagcccagagaaccccaggtgtacaccctgcctcccagccaggaagagatgaccaagaaccaggtgtccctgacctgcctggtgaaaggcttctaccccagcgacatcgccgtggagtgggaaagcaacggccagcccgagaacaattacaagacaacccctcccgtgctggatagcgatggcagcttctttctgtacagcagactgaccgtggacaagagcagatggcaggaaggcaacgtgttcagctgcagcgtgatgcacgaagccctgcacaaccactacacccagaagagcctgtccctgagcctgggcaag

SEQ ID NO: 39 - DNA, 1278 bp, Artificial Sequence
  nucleotide sequence of modified IL-7(MMMM) fused hyFc
atgttccacgtgagcttcagatacatcttcggcctgccccccctgatcctggtgctgctgcccgtggccagcagcatgatgatgatggactgcgacatcgagggcaaggacggcaagcagtacgagagcgtgctgatggtgagcatcgaccagctgctggacagcatgaaggagatcggcagcaactgcctgaacaacgagttcaacttcttcaagagacacatctgcgacgccaacaaggagggcatgttcctgttcagagccgccagaaagctgagacagttcctgaagatgaacagcaccggcgacttcgacctgcacctgctgaaggtgagcgagggcacaaccatcctgctgaactgcaccggccaggtgaagggcagaaagcccgccgccctgggcgaggcccagcccaccaagagcctggaggagaacaagagcctgaaggagcagaagaagctgaacgacctgtgcttcctgaagagactgctgcaggagatcaagacctgctggaacaagatcctgatgggcaccaaggagcacaggaacacaggcagaggcggcgaggagaagaagaaggagaaggagaaggaggagcaggaggaaagagagaccaagacccccgagtgccccagccacacccagcccctgggcgtgttcctgttccctcccaagcccaaggacaccctgatgatcagcagaacccccgaggtgacctgcgtggtcgtggatgtgagccaggaagatcccgaagtgcagttcaactggtacgtggatggcgtggaagtgcacaacgccaagaccaagcccagagaagagcagttcaactccacctacagagtggtgagcgtgctgaccgtgctgcaccaggactggctgaacggcaaggagtacaagtgcaaggtgtccaacaaaggcctgcccagctccatcgagaagaccatcagcaaagccaaaggccagcccagagaaccccaggtgtacaccctgcctcccagccaggaagagatgaccaagaaccaggtgtccctgacctgcctggtgaaaggcttctaccccagcgacatcgccgtggagtgggaaagcaacggccagcccgagaacaattacaagacaacccctcccgtgctggatagcgatggcagcttctttctgtacagcagactgaccgtggacaagagcagatggcaggaaggcaacgtgttcagctgcagcgtgatgcacgaagccctgcacaaccactacacccagaagagcctgtccctgagcctgggcaag

SEQ ID NO: 40 - protein, 4 aa, Artificial Sequence
  synthetic oligopeptide fragment
MMMM

SEQ ID NO: 41 - protein, 4 aa, Artificial Sequence
  synthetic oligopeptide fragment
MGGM

SEQ ID NO: 42 - protein, 4 aa, Artificial Sequence
  synthetic oligopeptide fragment
MMGG

SEQ ID NO: 43 - protein, 4 aa, Artificial Sequence
  synthetic oligopeptide fragment
GGMM

SEQ ID NO: 44 - protein, 4 aa, Artificial Sequence
  synthetic oligopeptide fragment
MGMG

SEQ ID NO: 45 - protein, 4 aa, Artificial Sequence
  synthetic oligopeptide fragment
GMMG

SEQ ID NO: 46 - protein, 4 aa, Artificial Sequence
  synthetic oligopeptide fragment
GGGM

SEQ ID NO: 47 - protein, 4 aa, Artificial Sequence
  synthetic oligopeptide fragment
MGGG

SEQ ID NO: 48 - protein, 4 aa, Artificial Sequence
  synthetic oligopeptide fragment
GMGG

SEQ ID NO: 49 - protein, 4 aa, Artificial Sequence
  synthetic oligopeptide fragment
GGMG

SEQ ID NO: 50 - protein, 5 aa, Artificial Sequence
  synthetic oligopeptide fragment
GGMMM

SEQ ID NO: 51 - protein, 5 aa, Artificial Sequence
  synthetic oligopeptide fragment
GGGMM

SEQ ID NO: 52 - protein, 5 aa, Artificial Sequence
  synthetic oligopeptide fragment
GGGGM

SEQ ID NO: 53 - protein, 5 aa, Artificial Sequence
  synthetic oligopeptide fragment
MGMMM

SEQ ID NO: 54 - protein, 5 aa, Artificial Sequence
  synthetic oligopeptide fragment
MGGMM

SEQ ID NO: 55 - protein, 5 aa, Artificial Sequence
  synthetic oligopeptide fragment
MGGGM

SEQ ID NO: 56 - protein, 5 aa, Artificial Sequence
  synthetic oligopeptide fragment
MMGMM

SEQ ID NO: 57 - protein, 5 aa, Artificial Sequence
  synthetic oligopeptide fragment
MMGGM

SEQ ID NO: 58 - protein, 5 aa, Artificial Sequence
  synthetic oligopeptide fragment
MMGGG

SEQ ID NO: 59 - protein, 5 aa, Artificial Sequence
  synthetic oligopeptide fragment
MMMGM

SEQ ID NO: 60 - protein, 5 aa, Artificial Sequence
  synthetic oligopeptide fragment
MGMGM

SEQ ID NO: 61 - protein, 5 aa, Artificial Sequence
  synthetic oligopeptide fragment
GMGMG

SEQ ID NO: 62 - protein, 5 aa, Artificial Sequence
  synthetic oligopeptide fragment
GMMMG

SEQ ID NO: 63 - protein, 5 aa, Artificial Sequence
  synthetic oligopeptide fragment
GGMGM

SEQ ID NO: 64 - protein, 5 aa, Artificial Sequence
  synthetic oligopeptide fragment
GGMMG

SEQ ID NO: 65 - protein, 5 aa, Artificial Sequence
  synthetic oligopeptide fragment
GMMGM

SEQ ID NO: 66 - protein, 5 aa, Artificial Sequence
  synthetic oligopeptide fragment
MGMMG

SEQ ID NO: 67 - protein, 5 aa, Artificial Sequence
  synthetic oligopeptide fragment
GMGGM

SEQ ID NO: 68 - protein, 5 aa, Artificial Sequence
  synthetic oligopeptide fragment
MMGMG

SEQ ID NO: 69 - protein, 5 aa, Artificial Sequence
  synthetic oligopeptide fragment
GMMGG

SEQ ID NO: 70 - protein, 5 aa, Artificial Sequence
  synthetic oligopeptide fragment
GMGGG

SEQ ID NO: 71 - protein, 5 aa, Artificial Sequence
  synthetic oligopeptide fragment
GGMGG

SEQ ID NO: 72 - protein, 5 aa, Artificial Sequence
  synthetic oligopeptide fragment
GGGGG

SEQ ID NO: 73 - protein, 501 aa, Artificial Sequence
  anti-PD-L1/CD3 bispecific antibody
EQKLISEEDLDIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKGGGGSGGGGSGGGGSEVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSSGGGGSEVQLVESGGGLVQPGKSLKLSCEASGFTFSGYGMHWVRQAPGRGLESVAYITSSSINIKYADAVKGRFTVSRDNAKNLLFLQMNILKSEDTAMYYCARFDWDKNYWGQGTMVTVSSGGGGSGGGGSGGGGSDIQMTQSPSSLPASLGDRVTINCQASQDISNYLNWYQQKPGKAPKLLIYYTNKLADGVPSRFSGSGSGRDSSFTISSLESEDIGSYYCQQYYNYPWTFGPGTKLEIKHHHHHHHV

(19) United States
(12) Patent Application Publication
(10) Pub. No.: US 2023/0057939 A1
(43) Pub. Date: Feb. 23, 2023

(54) METHOD OF TREATING A TUMOR WITH A COMBINATION OF IL-7 PROTEIN AND A BISPECIFIC ANTIBODY

(71) Applicant: NeoImmuneTech, Inc., Rockville, MD (US)

(72) Inventors: Seung-Woo LEE, Pohang-si (KR); Donghoon CHOI, Seongnam-si (KR); Byung Ha LEE, Rockville, MD (US); Se Hwan YANG, Seongnam-si (KR); Ji-Hae KIM, Pohang-si (KR); Sujeong PARK, Pohang-si (KR)

(73) Assignee: NeoImmuneTech, Inc., Rockville, MD (US)

(21) Appl. No.: 17/792,663

(22) PCT Filed: Jan. 12, 2021
(86) PCT No.: PCT/US2021/013074

§ 371 Date: Jul. 13, 2022

Related U.S. Application Data
(60) Provisional application No. 63/109,475, filed on Nov. 4, 2020.
(60) Provisional application No. 63/108,760, filed on Nov. 2, 2020.
(60) Provisional application No. 62/960,593, filed on Jan. 13, 2020.

(51) Int. Cl. A61K 38/20; A61P 35/00; A61P 1/00; A61K 39/395

(52) U.S. Cl. CPC A61K 38/2046; A61P 35/00; A61P 1/00; A61K 39/39558; A61K 39/3955; A61K 2039/545

(57) ABSTRACT

The present disclosure relates to methods of treating a cancer (or a tumor) with an IL-7 protein in combination with a multispecific (e.g., bispecific) antibody.

### CROSS-REFERENCE TO RELATED APPLICATION

This PCT application claims the priority benefit of U.S. Provisional Application Nos. 62/960,593, filed Jan. 13, 2020; 63/108,760, filed Nov. 2, 2020; and 63/109,475, filed Nov. 4, 2020, each of which is herein incorporated by reference in its entirety.

### REFERENCE TO SEQUENCE LISTING SUBMITTED ELECTRONICALLY VIA EFS-WEB

The content of the electronically submitted sequence listing in ASCII text file (Name: 4241_004PC03_SequenceListing_ST25.txt; Size: 87,920 bytes; and Date of Creation: Jan. 12, 2021) filed with the application is herein incorporated by reference in its entirety.

### BACKGROUND OF THE DISCLOSURE

Cancer immunotherapy (e.g., antibodies) has become well-established in recent years and is now one of the more successful treatment options available for many cancer patients. Scott, A. M., et al., Cancer Immun 12:14 (2012). Aside from targeting antigens that are involved in cancer cell proliferation and survival, antibodies can also activate or antagonize immunological pathways that are important in cancer immune surveillance. And, intensive efforts have led to the successful development of several immune checkpoint pathway inhibitors, some of which have been approved by the Food and Drug Administration, e.g., anti-CTLA-4 antibody: ipilimumab (YERVOY®); anti-PD-1 antibody: nivolumab (OPDIVO®), pembrolizumab (KEYTRUDA®), cemiplimab (LIBTAYO®); and anti-PD-L1 antibody: atezolizumab (TECENTRIQ®), durvalumab (IMFINZI®), avelumab (BAVENCIO®).

Despite such advances, patients with certain malignant tumors (e.g., metastatic or refractory solid tumors) continue to have very poor prognosis. Only a subset of such patients actually experience long-term cancer remission, with many patients either not responding or initially responding but eventually developing resistance to the antibodies. Sharma, P., et al., Cell 168(4): 707-723 (2017). Moreover, many cancer patients are lymphopenic, as many of the available standard of care cancer treatments (e.g., chemotherapy and radiation therapy) are known to cause lymphopenia. Grossman, S. A., et al., J Natl Compr Canc Netw 13(10):1225-31 (2015). Checkpoint inhibitors, such as anti-PD-1 antibodies, have been shown to have limited efficacy in such cancer patients. Yarchoan, M., et al., J Clin Oncol 35:e14512 (2017). Accordingly, there remains a need for new treatment options with acceptable safety profile and high efficacy in cancer patients, including those with lymphopenia.

SUMMARY OF THE DISCLOSURE

Provided herein is a method of treating a tumor in a subject in need thereof, comprising administering to the subject an effective amount of a modified IL-7 protein and a bispecific antibody. In some aspects, a tumor volume is reduced in the subject after the administration compared to a reference tumor volume (e.g., tumor volume in the subject prior to administration and/or tumor volume in a subject after administration of either the modified IL-7 protein or the bispecific antibody alone). In certain aspects, the tumor volume is reduced by at least about 5%, at least about 10%, at least about 20%, at least about 30%, at least about 40%, at least about 50%, at least about 60%, at least about 70%, at least about 80%, at least about 90%, or at least about 100% after the administration compared to the reference tumor volume.

In some aspects, a body weight of the subject is not decreased after the administration compared to a reference body weight (e.g., body weight of the subject prior to administration and/or body weight of a subject after administration of either the modified IL-7 protein or the bispecific antibody alone). In certain aspects, the body weight of the subject is not decreased by more than about 1%, more than about 2%, more than about 3%, more than about 4%, more than about 5%, more than about 6%, more than about 7%, more than about 8%, more than about 9%, or more than about 10% after the administration compared to the reference body weight.

Also provided herein is a method of enhancing an anti-tumor activity of a bispecific antibody in a subject in need thereof, comprising administering to the subject an effective amount of a bispecific antibody in combination with a modified IL-7 protein. In some aspects, the anti-tumor activity comprises a reduction in tumor volume and/or lack of loss of body weight in the subject.

In some aspects, the tumor volume is reduced by at least about 5%, at least about 10%, at least about 20%, at least about 30%, at least about 40%, at least about 50%, at least about 60%, at least about 70%, at least about 80%, at least about 90%, or at least about 100% after the administration compared to a reference tumor volume (e.g., tumor volume in the subject prior to administration and/or tumor volume in a subject after administration of either the modified IL-7 protein or the bispecific antibody alone).

In some aspects, the body weight of the subject is not decreased by more than about 1%, more than about 2%, more than about 3%, more than about 4%, more than about 5%, more than about 6%, more than about 7%, more than about 8%, more than about 9%, or more than about 10% after the administration compared to a reference body weight (e.g., body weight of the subject prior to administration and/or body weight of a subject after administration of either the modified IL-7 protein or the bispecific antibody alone).

In some aspects, the modified IL-7 protein comprises an oligopeptide consisting of 1 to 10 amino acid residues. In certain aspects, the oligopeptide comprises methionine (M), glycine (G), methionine-methionine (MM), glycine-glycine (GG), methionine-glycine (MG), glycine-methionine (GM), methionine-methionine-methionine (MMM), methionine-methionine-glycine (MMG), methionine-glycine-methionine (MGM), glycine-methionine-methionine (GMM), methionine-glycine-glycine (MGG), glycine-methionine-glycine (GMG), glycine-glycine-methionine (GGM), glycine-glycine-glycine (GGG), methionine-glycine-glycine-methionine (MGGM) (SEQ ID NO: 41), methionine-methionine-glycine-glycine (MMGG) (SEQ ID NO: 42), glycine-glycine-methionine-methionine (GGMM) (SEQ ID NO: 43), methionine-glycine-methionine-glycine (MGMG) (SEQ ID NO: 44), glycine-methionine-methionine-glycine (GMMG) (SEQ ID NO: 45), glycine-glycine-glycine-methionine (GGGM) (SEQ ID NO: 46), methionine-glycine-glycine-glycine (MGGG) (SEQ ID NO: 47), glycine-methionine-glycine-glycine (GMGG) (SEQ ID NO: 48), glycine-glycine-methionine-glycine (GGMG) (SEQ ID NO: 49), glycine-glycine-methionine-methionine-methionine (GGMMM) (SEQ ID NO: 50), glycine-glycine-glycine-methionine-methionine (GGGMM) (SEQ ID NO: 51), glycine-glycine-glycine-glycine-methionine (GGGGM) (SEQ ID NO: 52), methionine-glycine-methionine-methionine-methionine (MGMMM) (SEQ ID NO: 53), methionine-glycine-glycine-methionine-methionine (MGGMM) (SEQ ID NO: 54), methionine-glycine-glycine-glycine-methionine (MGGGM) (SEQ ID NO: 55), methionine-methionine-glycine-methionine-methionine (MMGMM) (SEQ ID NO: 56), methionine-methionine-glycine-glycine-methionine (MMGGM) (SEQ ID NO: 57), methionine-methionine-glycine-glycine-glycine (MMGGG) (SEQ ID NO: 58), methionine-methionine-methionine-glycine-methionine (MMMGM) (SEQ ID NO: 59), methionine-glycine-methionine-glycine-methionine (MGMGM) (SEQ ID NO: 60), glycine-methionine-glycine-methionine-glycine (GMGMG) (SEQ ID NO: 61), glycine-methionine-methionine-methionine-glycine (GMMMG) (SEQ ID NO: 62), glycine-glycine-methionine-glycine-methionine (GGMGM) (SEQ ID NO: 63), glycine-glycine-methionine-methionine-glycine (GGMMG) (SEQ ID NO: 64), glycine-methionine-methionine-glycine-methionine (GMMGM) (SEQ ID NO: 65), methionine-glycine-methionine-methionine-glycine (MGMMG) (SEQ ID NO: 66), glycine-methionine-glycine-glycine-methionine (GMGGM) (SEQ ID NO: 67), methionine-methionine-glycine-methionine-glycine (MMGMG) (SEQ ID NO: 68), glycine-methionine-methionine-glycine-glycine (GMMGG) (SEQ ID NO: 69), glycine-methionine-glycine-glycine-glycine (GMGGG) (SEQ ID NO: 70), glycine-glycine-methionine-glycine-glycine (GGMGG) (SEQ ID NO: 71), glycine-glycine-glycine-glycine-glycine (GGGGG) (SEQ ID NO: 72), or combinations thereof. In certain aspects, the oligopeptide is methionine-glycine-methionine (MGM).

In some aspects, the modified IL-7 protein comprises a half-life extending moiety. In certain aspects, the half-life extending moiety comprises an Fc, albumin, an albumin-binding polypeptide, Pro/Ala/Ser (PAS), a C-terminal peptide (CTP) of the β subunit of human chorionic gonadotropin, polyethylene glycol (PEG), long unstructured hydrophilic sequences of amino acids (XTEN), hydroxyethyl starch (HES), an albumin-binding small molecule, or a combination thereof.

In some aspects, the half-life extending moiety is an Fc. In further aspects, the Fc is a hybrid Fc, comprising a hinge region, a CH2 domain, and a CH3 domain, wherein the hinge region comprises a human IgD hinge region, wherein the CH2 domain comprises a part of human IgD CH2 domain and a part of human IgG4 CH2 domain, and wherein the CH3 domain comprises a part of human IgG4 CH3 domain.

In some aspects, the modified IL-7 protein comprises an amino acid sequence having a sequence identity of at least about 70%, at least about 75%, at least about 80%, at least about 85%, at least about 90%, at least about 95%, at least about 96%, at least about 97%, at least about 98%, at least about 99%, or about 100% to SEQ ID NOs: 1-6 and 15-25.

In some aspects, the bispecific antibody comprises a T-cell engager (e.g., bispecific T-cell engager (BsAb) antibody), dual-affinity retargeting molecule (DART), CrossMAb antibody, DutaMab™ antibody, DuoBody antibody, Triomab, TandAb, bispecific NanoBody, Tandem scFv, diabody, single chain diabody, HSA body, (scFv)2 HSA Antibody, scFv-IgG antibody, Dock and Lock bispecific antibody, DVD-IgG antibody, TBTI DVD-IgG, IgG-fynomer, Tetravalent bispecific tandem IgG antibody, dual-targeting domain antibody, chemically linked bispecific (Fab′)2 molecule, crosslinked mAb, Dual-action Fab IgG (DAF-IgG), orthoFab-IgG, bispecific CovX-Body, bispecific hexavalent trimerbody, 2 scFv linked to diphtheria toxin, ART-Ig, IgM T-cell engager, or combinations thereof. In certain aspects, the bispecific antibody comprises a T-cell engager (e.g., bispecific T-cell engager (BiTE) antibody).

In some aspects, the bispecific antibody binds to a tumor antigen and an antigen expressed on an immune cell. In certain aspects, the antigen expressed on an immune cell comprises CD2, CD3, CD4, CD5, CD8, CD11b, CD14, CD16, CD19, CD28, CD32, CD45, CD56, CD64, KLRG-1, NKG2D, NKp30, DNAM-1, or combinations thereof. In further aspects, the tumor antigen comprises guanylate cyclase C (GC-C), epidermal growth factor receptor (EGFR or erbB-1), human epidermal growth factor receptor 2 (HER2 or erbB2), erbB-3, erbB-4, MUC-1, melanoma-associated chondroitin sulfate proteoglycan (MCSP), mesothelin (MSLN), folate receptor 1 (FOLR1), CD4, CD19, CD20, CD22, CD30, CD33, CD38, CD44, CD44v6, CD44v7/8, CD70, CD123, CD138, CD171, CEA, CSPG4, CXCRS, c-Met, HERV-envelope protein, eriostin, Bigh3, SPARC, BCR, CD79, CD37, EGFRvIII, EGP2, EGP40, IGFr, L1CAM, AXL, Tissue Factor (TF), CD74, EpCAM, EphA2, MRP3cadherin 19 (CDH19), epidermal growth factor 2 (HER2), 5T4, 8H9, αᵥβ₆ integrin, BCMA, B7-H3, B7-H6, CAIX, CA9, FAP, FBP, fetal AchR, FRcc, GD2, GD3, Glypican-1 (GPC1), Glypican-2 (GPC2), Glypican-3 (GPC3), HLA-A1+MAGE1, HLA-A1+NY-ESO-1, IL-13Rcc2, Lewis-Y, KDR, MCSP, Mesothelin, Muc1, Muc16, NCAM, NKG2D ligands, NY-ESO-1, PRAIVIE, PSC1, PSCA, PSMA, ROR1, ROR2, SP17, surviving, TAG72, TEMs, carcinoembryonic antigen, HMW-MAA, VEGF, CLDN18.2, or combinations thereof.

In some aspects, the tumor antigen comprises an immune checkpoint molecule. In certain aspects, the immune checkpoint molecule comprises a PD-1 ligand (e.g., PD-L1), LAG3 ligand, TIM-3 ligand (e.g., galectin 9), CTLA-4 ligand (e.g., CD28), OX40 ligand, CD28 ligand (e.g., B7H3 or B7H4), or combinations thereof.

In some aspects, the immune cell comprises a T-cell. In certain aspects, the T-cell comprises a tumor-infiltrating lymphocyte (TIL).

In some aspects, the modified IL-7 protein and the bispecific antibody are administered concurrently. In certain aspects, the modified IL-7 protein and the bispecific antibody are administered sequentially. In further aspects, the IL-7 protein is administered to the subject prior to administering the bispecific antibody.

In some aspects, the modified IL-7 protein is administered at a dose of greater than about 600 μg/kg, greater than about 700 μg/kg, greater than about 800 μg/kg, greater than about 900 μg/kg, greater than about 1,000 μg/kg, greater than about 1,100 μg/kg, greater than about 1,200 μg/kg, greater than about 1,300 μg/kg, greater than about 1,400 μg/kg, greater than about 1,500 μg/kg, greater than about 1,600 μg/kg, greater than about 1,700 μg/kg, greater than about 1,800 μg/kg, greater than about 1,900 μg/kg, or greater than about 2,000 μg/kg.

In some aspects, the modified IL-7 protein is administered at a dose of between about 610 μg/kg and about 1,200 μg/kg, between about 650 μg/kg and about 1,200 μg/kg, between about 700 μg/kg and about 1,200 μg/kg, between about 750 μg/kg and about 1,200 μg/kg, between about 800 μg/kg and about 1,200 μg/kg, between about 850 μg/kg and about 1,200 μg/kg, between about 900 μg/kg and about 1,200 μg/kg, between about 950 μg/kg and about 1,200 μg/kg, between about 1,000 μg/kg and about 1,200 μg/kg, between about 1,050 μg/kg and about 1,200 μg/kg, between about 1,100 μg/kg and about 1,200 μg/kg, between about 1,200 μg/kg and about 2,000 μg/kg, between about 1,300 μg/kg and about 2,000 μg/kg, between about 1,500 μg/kg and about 2,000 μg/kg, between about 1,700 μg/kg and about 2,000 μg/kg, between about 610 μg/kg and about 1,000 μg/kg, between about 650 μg/kg and about 1,000 μg/kg, between about 700 μg/kg and about 1,000 μg/kg, between about 750 μg/kg and about 1,000 μg/kg, between about 800 μg/kg and about 1,000 μg/kg, between about 850 μg/kg and about 1,000 μg/kg, between about 900 μg/kg and about 1,000 μg/kg, or between about 950 μg/kg and about 1,000 μg/kg.

In some aspects, the modified IL-7 protein is administered at a dose of between about 700 μg/kg and about 900 μg/kg, between about 750 μg/kg and about 950 μg/kg, between about 700 μg/kg and about 850 μg/kg, between about 750 μg/kg and about 850 μg/kg, between about 700 μg/kg and about 800 μg/kg, between about 800 μg/kg and about 900 μg/kg, between about 750 μg/kg and about 850 μg/kg, or between about 850 μg/kg and about 950 μg/kg.

In some aspects, the modified IL-7 protein is administered at a dose of about 650 μg/kg, about 680 μg/kg, about 700 μg/kg, about 720 μg/kg, about 740 μg/kg, about 750 μg/kg, about 760 μg/kg, about 780 μg/kg, about 800 μg/kg, about 820 μg/kg, about 840 μg/kg, about 850 μg/kg, about 860 μg/kg, about 880 μg/kg, about 900 μg/kg, about 920 μg/kg, about 940 μg/kg, about 950 μg/kg, about 960 μg/kg, about 980 μg/kg, about 1000 μg/kg, about 1100 μg/kg, about 1200 μg/kg, about 1,300 μg/kg, about 1,400 μg/kg, about 1,440 μg/kg, about 1,500 μg/kg, about 1,600 μg/kg, about 1,700 μg/kg, about 1,800 μg/kg, about 1,900 μg/kg, or about 2,000 μg/kg.

In some aspects, the modified IL-7 protein is administered at a dosing frequency of once a week, once in two weeks, once in three weeks, once in four weeks, once in five weeks, once in six weeks, once in seven weeks, once in eight weeks, once in nine weeks, once in 10 weeks, once in 11 weeks, or once in 12 weeks.

In some aspects, the bispecific antibody is administered to the subject at a dose of about 0.1 mg/kg to about 20 mg/kg.

In some aspects, the modified IL-7 protein is administered to the subject parenthetically, intramuscularly, subcutaneously, ophthalmic, intravenously, intraperitoneally, intradermally, intraorbitally, intracerebrally, intracranially, intraspinally, intraventricular, intrathecally, intracistemally, intracapsularly, or intratumorally. In some aspects, the bispecific antibody is administered to the subject parenthetically, intramuscularly, subcutaneously, ophthalmic, intravenously, intraperitoneally, intradermally, intraorbitally, intracerebrally, intracranially, intraspinally, intraventricular, intrathecally, intracistemally, intracapsularly, or intratumorally. In certain aspects, the bispecific antibody is administered intratumorally.

In some aspects, a method of treating a tumor in a subject or a method of enhancing an anti-tumor activity of a bispecific antibody in a subject disclosed herein further comprises administering at least one additional therapeutic agent to the subject.

In some aspects, the tumor is derived from a cancer comprising a breast cancer, head and neck cancer, uterine cancer, brain cancer, skin cancer, renal cancer, lung cancer, colorectal cancer, prostate cancer, liver cancer, bladder cancer, kidney cancer, pancreatic cancer, thyroid cancer, esophageal cancer, eye cancer, stomach (gastric) cancer, gastrointestinal cancer, ovarian cancer, carcinoma, sarcoma, leukemia, lymphoma, myeloma, or a combination thereof.

### BRIEF DESCRIPTION OF THE FIGURES

FIGS. 1A, 1B, and 1C show the effect of anti-PD-L1/CD3 bi-specific T-cell engager (“BsAb”) administration on a mouse adenocarcinoma model. FIG. 1A is a diagram of the schedule of tumor inoculation and treatment administration. FIGS. 1B and 1C are graphs showing comparative results of tumor volume (mm³) and body weight, respectively, in the different treatment groups after treatment with the BsAb. The treatment groups included: (1) control group phosphate buffered saline; (2) 0.2 μg BsAb; (3) 1.0 μg BsAb; and (4) 5.0 μg BsAb. The data are shown as mean±S.E.M. All comparisons were performed using two-way ANOVA with Bonferroni posts-tests. “*,” “**,” and “***” indicate a statistically significant difference (p<0.05, p<0.001, and p<0.0001, respectively) compared to the control animals.

FIGS. 1D, 1E, and 1F show the effect of anti-PD-L1/CD3 BsAb on the cytotoxicity and activation status of CD8+ T cells cultured in vitro with either wild-type or PD-L1-deficient MC38 tumor cells. FIG. 1D provides comparison of PD-L1 expression on the wild-type (left graph) and PD-L1-deficient (right graph) MC38 tumor cells as measured by flow cytometry. FIG. lE provides a comparison of the ability of the CD8+ T cells to kill the tumor cells (i.e., cytotoxicity) at varying concentrations of the BsAb. Cytotoxicity was calculated as follows: [1−live target cells(sample)/live target cells(control)]×100. FIG. 1F provides a comparison of CD69 (top row) and CD25 (bottom row) (i.e., activation markers) expression on the CD8+ T cells when cultured in varying concentrations of BsAb (i.e., 0 ng, 0.1 ng, 1 ng, 10 ng, and 100 ng) with: (i) no tumor cells (left column); (ii) wild-type MC-38 tumor cells (middle column); and (iii) PD-L1-deficient MC-38 tumor cells (right column).

FIGS. 2A, 2B, and 2C show the effect of IL-7 protein administration on the anti-tumor effects of anti-PD-L1/CD3 BsAb administered intravenously in a mouse adenocarcinoma model. FIG. 2A is a diagram of the schedule of tumor inoculation and treatment administration. FIGS. 2B and 2C are graphs showing comparative results of tumor volume (mm³) and body weight (% of initial), respectively, in the different treatment groups. The treatment groups included: (1) buffer+PBS; (2) IL-7 protein+PBS; (3) Buffer+1.0 μg anti-PD-L1/CD3 BsAb; (4) IL-7 protein+0.04 μg of anti-PD-L1/CD3 BsAb; (5) IL-7 protein+0.2 μg of anti-PD-L1/CD3 BsAb; and (6) IL-7 protein+1.0 μg Anti-PD-L1/CD3 BsAb. The data are shown as mean±S.E.M. All comparisons were performed using two-way ANOVA with Bonferroni posts-tests. “*,” “**,” and “***” indicate a statistically significant difference (p<0.05, p<0.001, and p<0.0001, respectively) compared to the control animals.

FIGS. 3A, 3B, 3C, 3D, 3E, and 3F provide comparison of tumor-infiltrating T cells observed in a mouse adenocarcinoma model treated with one of the following: (1) buffer+PBS; (2) IL-7 protein+PBS; (3) Buffer+0.2 μg anti-PD-L1/CD3 BsAb; and (4) IL-7 protein+0.2 μg of anti-PD-L1/CD3 BsAb. The legend for the groups shown in FIGS. 3B-3F is provided in FIG. 3B. FIG. 3A provides a schematic of the tumor inoculation and treatment administration schedule. FIG. 3B provides a comparison of the frequency of the following T cells in the tumors of the animals from the different treatment groups: (i) CD8+ T cells, (ii) FoxP3− CD4+ helper T cells; and (iii) Foxp3+ CD4+ regulatory T cells. The frequency of the different T cell populations is shown as a percentage of total CD45+ cells. FIG. 3C provides a comparison of FoxP3+ cells among total CD4+ T cells observed in the tumors of the animals from the different treatment groups. FIG. 3D provides a ratio of the CD8+ T cells to the regulatory T cells for the animals from the different treatment groups. FIG. 3E provides a comparison of the frequency of PD-1− cells among total CD8+ T cells observed in the tumors of the different animals. FIG. 3F shows the frequency of Granzyme B expressing cells among PD-1+ or PD-1− CD8+ T cells observed in the tumors of animals from the different treatment groups. Data are represented as mean +SD. Statistical significance was analyzed by one-way ANOVA with Bonferroni's multiple comparisons. *P<0.05;**P<0.001;***P<0.0001.

FIGS. 4A, 4B, and 4C show the effect of IL-7 protein administration on the anti-tumor effects of anti-PD-L1/CD3 BsAb administered intratumorally in a mouse adenocarcinoma model. FIG. 4A is a diagram of the schedule of tumor inoculation and treatment administration. FIGS. 4B and 4C are graphs showing comparative results of tumor volume (mm³) and body weight (% of initial), respectively, in the different treatment groups. The treatment groups included: (1) buffer +PBS; (2) IL-7 protein+PBS; (3) Buffer+1 μg anti-PD-L1/CD3 BsAb; (4) IL-7 protein+0.2 μg of anti-PD-L1/CD3 BsAb; and (5) IL-7 protein+1.0 μg Anti-PD-L1/CD3 BsAb (Group 5). The data are shown as mean±S.E.M. All comparisons were performed using two-way ANOVA with Bonferroni posts-tests. “*,” “**,” and “***” indicate a statistically significant difference (p<0.05, p<0.001, and p<0.0001, respectively) compared to the control animals.

FIGS. 5A, 5B, and 5C show the effect of anti-PD-L1/CD3 BsAb after intratumoral administration in a mouse adenocarcinoma model. FIG. 5A provides a schematic of tumor inoculation and BsAb administration schedule. FIGS. 5B and 5C are graphs showing comparative results of tumor volume (mm³) and body weight (% of initial), respectively, in the different treatment groups. The treatment groups included: (1) PBS alone; (2) 0.2 μg of anti-PD-L1/CD3 BsAb; (3) 1 μg of anti-PD-L1/CD3 BsAb; and (4) 5 μg of anti-PD-L1/CD3 BsAb. The legend for the groups shown in FIGS. 5B and 5C is provided in FIG. 5B. Data are represented as mean±SEM. Statistical significance was analyzed by two-way ANOVA with Bonferroni's multiple comparisons for tumor growth graphs. *P<0.05;**P<0.001;***P<0.0001.

### DETAILED DESCRIPTION OF THE INVENTION

### I. Definitions

In order that the present disclosure can be more readily understood, certain terms are first defined. As used in this application, except as otherwise expressly provided herein, each of the following terms shall have the meaning set forth below. Additional definitions are set forth throughout the application.

Throughout this disclosure, the term “a” or “an” entity refers to one or more of that entity; for example, “an antibody,” is understood to represent one or more antibodies. As such, the terms “a” (or “an”), “one or more,” and “at least one” can be used interchangeably herein.

Furthermore, “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

It is understood that wherever aspects are described herein with the language “comprising,” otherwise analogous aspects described in terms of “consisting of” and/or “consisting essentially of” are also provided.

Unless defined otherwise, all technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary Of Biochemistry And Molecular Biology, Revised, 2000, Oxford University Press, provide one of skill with a general dictionary of many of the terms used in this disclosure.

Units, prefixes, and symbols are denoted in their Système International de Unites (SI) accepted form. Numeric ranges are inclusive of the numbers defining the range. Unless otherwise indicated, amino acid sequences are written left to right in amino to carboxy orientation. The headings provided herein are not limitations of the various aspects of the disclosure, which can be had by reference to the specification as a whole. Accordingly, the terms defined immediately below are more fully defined by reference to the specification in its entirety.

The term “about” is used herein to mean approximately, roughly, around, or in the regions of. When the term “about” is used in conjunction with a numerical range, it modifies that range by extending the boundaries above and below the numerical values set forth. In general, the term “about” can modify a numerical value above and below the stated value by a variance of, e.g., 10 percent, up or down (higher or lower).

As used herein, “administering” refers to the physical introduction of a therapeutic agent or a composition comprising a therapeutic agent to a subject, using any of the various methods and delivery systems known to those skilled in the art. The different routes of administration for a therapeutic agent described herein include intravenous, intraperitoneal, intramuscular, subcutaneous, spinal or other parenteral routes of administration, for example by injection or infusion. The phrase “parenteral administration” as used herein means modes of administration other than enteral and topical administration, usually by injection, and includes, without limitation, intravenous, intraperitoneal, intramuscular, intraarterial, intrathecal, intralymphatic, intralesional, intracapsular, intraorbital, intracardiac, intradermal, transtracheal, intratracheal, pulmonary, subcutaneous, subcuticular, intraarticular, subcapsular, subarachnoid, intraventricle, intravitreal, epidural, and intrasternal injection and infusion, as well as in vivo electroporation. Alternatively, a therapeutic agent described herein can be administered via a non-parenteral route, such as a topical, epidermal, or mucosal route of administration, for example, intranasally, orally, vaginally, rectally, sublingually, or topically. Administering can also be performed, for example, once, a plurality of times, and/or over one or more extended periods.

As used herein, the term “antigen” refers to any natural or synthetic immunogenic substance, such as a protein, peptide, or hapten.

The terms “antibody” and “antibodies” are terms of art and can be used interchangeably herein and refer to a molecule with an antigen binding site that specifically binds an antigen. The terms as used to herein include whole antibodies and any antigen binding fragments (i.e., “antigen-binding portions”) or single chains thereof. An “antibody” refers, in some aspects, to a glycoprotein comprising at least two heavy (H) chains and two light (L) chains inter-connected by disulfide bonds, or an antigen-binding portion thereof. In another aspect, an “antibody” refers to a single chain antibody comprising a single variable domain, e.g., VHH domain. Each heavy chain is comprised of a heavy chain variable region (abbreviated herein as VH) and a heavy chain constant region. In certain naturally-occurring antibodies, the heavy chain constant region is comprised of three domains, CH1, CH2 and CH3. In certain naturally-occurring antibodies, each light chain is comprised of a light chain variable region (abbreviated herein as VL) and a light chain constant region. The light chain constant region is comprised of one domain, CL.

The VH and VL regions can be further subdivided into regions of hypervariability, termed complementarity determining regions (CDRs), interspersed with regions that are more conserved, termed framework regions (FR). Each VH and VL comprises three CDRs and four FRs, arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies can mediate the binding of the immunoglobulin to host tissues or factors, including various cells of the immune system (e.g., effector cells) and the first component (C1q) of the classical complement system.

Antibodies typically bind specifically to their cognate antigen with high affinity, reflected by a dissociation constant (K_{D}) of 10⁻⁵ to 10⁻¹¹M or less. Any K_{D} greater than about 10⁻⁴M is generally considered to indicate nonspecific binding. As used herein, an antibody that “binds specifically” to an antigen refers to an antibody that binds to the antigen and substantially identical antigens with high affinity, which means having a K_{D} of 10⁻⁷M or less, 10⁻⁸M or less, 5×10⁻⁹M or less, or between 10⁻⁸M and 10⁻¹⁰M or less, but does not bind with high affinity to unrelated antigens. An antigen is “substantially identical” to a given antigen if it exhibits a high degree of sequence identity to the given antigen, for example, if it exhibits at least 80%, at least 90%, at least 95%, at least 97%, or at least 99% sequence identity to the sequence of the given antigen. By way of example, an antibody that binds specifically to PD-1 can, in certain aspects, also have cross-reactivity with PD-1 antigens from certain primate species (e.g., cynomolgus anti-PD-1 antibody), but cannot cross-react with PD-1 molecules from other species or with a molecule other than PD-1.

An immunoglobulin can be derived from any of the commonly known isotypes, including but not limited to IgA, secretory IgA, IgG and IgM. IgG subclasses are also well known to those in the art and include but are not limited to human IgG1, IgG2, IgG3 and IgG4. “Isotype” refers to the antibody class or subclass (e.g., IgM or IgG1) that is encoded by the heavy chain constant region genes. In certain aspects, one or more amino acids of the isotype can be mutated to alter effector function. The term “antibody” includes, by way of example, both naturally occurring and non-naturally occurring Abs; monoclonal and polyclonal Abs; chimeric and humanized Abs; human or nonhuman Abs; wholly synthetic Abs; and single chain antibodies. A nonhuman antibody can be humanized by recombinant methods to reduce its immunogenicity in man. Where not expressly stated, and unless the context indicates otherwise, the term “antibody” also includes an antigen-binding fragment or an antigen-binding portion of any of the aforementioned immunoglobulins, and includes a monovalent and a divalent fragment or portion, and a single chain antibody.

An “isolated antibody” refers to an antibody that is substantially free of other antibodies having different antigenic specificities (e.g., an isolated antibody that binds specifically to PD-1 is substantially free of antibodies that bind specifically to antigens other than PD-1). An isolated antibody that binds specifically to PD-1 can, however, have cross-reactivity to other antigens, such as PD-1 molecules from different species. Moreover, an isolated antibody can be substantially free of other cellular material and/or chemicals.

The term “monoclonal antibody” (“mAb”) refers to a non-naturally occurring preparation of antibody molecules of single molecular composition, i.e., antibody molecules whose primary sequences are essentially identical, and which exhibits a single binding specificity and affinity for a particular epitope. A mAb is an example of an isolated antibody. MAbs can be produced by hybridoma, recombinant, transgenic or other techniques known to those skilled in the art.

A “human” antibody (HuMAb) refers to an antibody having variable regions in which both the framework and CDR regions are derived from human germline immunoglobulin sequences. Furthermore, if the antibody contains a constant region, the constant region also is derived from human germline immunoglobulin sequences. The human antibodies of the invention can include amino acid residues not encoded by human germline immunoglobulin sequences (e.g., mutations introduced by random or site-specific mutagenesis in vitro or by somatic mutation in vivo). However, the term “human antibody,” as used herein, is not intended to include antibodies in which CDR sequences derived from the germline of another mammalian species, such as a mouse, have been grafted onto human framework sequences. The terms “human” antibodies and “fully human” antibodies and are used synonymously.

A “humanized antibody” refers to an antibody in which some, most or all of the amino acids outside the CDR domains of a non-human antibody are replaced with corresponding amino acids derived from human immunoglobulins. In one aspect of a humanized form of an antibody, some, most or all of the amino acids outside the CDR domains have been replaced with amino acids from human immunoglobulins, whereas some, most or all amino acids within one or more CDR regions are unchanged. Small additions, deletions, insertions, substitutions or modifications of amino acids are permissible as long as they do not abrogate the ability of the antibody to bind to a particular antigen. A “humanized” antibody retains an antigenic specificity similar to that of the original antibody.

A “chimeric antibody” refers to an antibody in which the variable regions are derived from one species and the constant regions are derived from another species, such as an antibody in which the variable regions are derived from a mouse antibody and the constant regions are derived from a human antibody.

As used herein, the term “multispecific antibody” refers to an antibody having the ability to bind to multiple (e.g., more than one) distinct epitopes. As used herein, the term “bispecific antibody” refers to a multispecific antibody that is capable of binding to two distinct epitopes. In some aspects, the multiple distinct epitopes are on a single antigen (e.g., the bispecific antibody binds to two different regions of a single protein). In other aspects, the multiple distinct epitopes are on different antigens (e.g., the bispecific antibody binds to an epitope on Protein A and an epitope on Protein B). Unless indicated otherwise, the terms “multispecific antibody” and “bispecific antibody” are used interchangeably in the present disclosure. Accordingly, disclosure relating to bispecific antibodies can equally apply to multispecific antibodies.

Non-limiting examples of bispecific antibodies that can be used with the present disclosure include a T-cell engager (e.g., bispecific T-cell engager (BsAb) antibody), dual-affinity retargeting molecule (DART), CrossMAb antibody, DutaMab™ antibody, DuoBody antibody, Triomab, TandAb, bispecific NanoBody, Tandem scFv, diabody, single chain diabody, HSA body, (scFv)2 HSA Antibody, scFv-IgG antibody, Dock and Lock bispecific antibody, DVD-IgG antibody, TBTI DVD-IgG, IgG-fynomer, Tetravalent bispecific tandem IgG antibody, dual-targeting domain antibody, chemically linked bispecific (Fab′)2 molecule, crosslinked mAb, Dual-action Fab IgG (DAF-IgG), orthoFab-IgG, bispecific CovX-Body, bispecific hexavalent trimerbody, 2 scFv linked to diphtheria toxin, ART-Ig, IgM T-cell engager, or combinations thereof.

Bispecific antibodies can be made by a variety of methods known in the art, e.g., disulfide cleavage and reformation of mixtures of whole IgG or F(ab′)2 fragments, fusions of more than one hybridoma to form polyomas that produce antibodies having more than one specificity, and by genetic-engineering. Bispecific antibodies have been prepared by oxidative cleavage of Fab′ fragments resulting from reductive cleavage of different antibodies. This is advantageously carried out by mixing two different F(ab′)2 fragments produced by pepsin digestion of two different antibodies, reductive cleavage to form a mixture of Fab′ fragments, followed by oxidative reformation of the disulfide linkages to produce a mixture of F(ab′)2 fragments including bispecific antibodies containing a Fab′ portion specific to each of the original epitopes (e.g., an antigen expressed on an immune cell and a tumor antigen disclosed herein). General techniques for the preparation of multivalent antibodies may be found, for example, in Nisonhoff et al., Arch Biochem. Biophys. 93: 470 (1961), Hammerling et al., J. Exp. Med. 128: 1461 (1968), and U.S. Pat. No. 4,331,647.

Alternatively, such bispecific antibodies can be produced by fusing two hybridoma cell lines that produce an antibody against an antigen on an immune cell (e.g., CD3) and an antibody against a tumor antigen (e.g., PD-L1). Techniques for producing tetradomas are described, for example, by Milstein et al., Nature 305: 537 (1983) and Pohl et al., Int. J. Cancer 54: 418 (1993).

Finally, such bispecific antibodies can be produced by genetic engineering. For example, plasmids containing DNA coding for variable domains of an antibody against an antigen on an immune cell (e.g., anti-CD3) can be introduced into hybridomas that secrete anti-tumor antigen antibodies. The resulting “transfectomas” produce bispecific antibodies that bind the antigen on an immune cell and a tumor antigen. Alternatively, chimeric genes can be designed that encode both the binding domain against an antigen expressed on immune cells and a tumor antigen. General techniques for producing bispecific antibodies by genetic engineering are described, for example, by Songsivilai et al., Biochem. Biophys. Res. Commun. 164: 271 (1989); Traunecker et al., EMBO J. 10: 3655 (1991); and Weiner et al., J. Immunol. 147: 4035 (1991).

A higher order multivalent, multispecific molecule can be obtained by adding various antibody components to a bispecific antibody, produced as above. For example, a bispecific antibody can be reacted with 2-iminothiolane to introduce one or more sulfhydryl groups for use in coupling the bispecific antibody to a further antibody derivative that binds an the same or a different epitope of the target antigen, using the bis-maleimide activation procedure described above. These techniques for producing multivalent antibodies are well known to those of skill in the art. See, for example, U.S. Pat. No. 4,925,648, and Goldenberg, international publication No. WO 92/19273, which are incorporated by reference.

As used herein, the term “epitope” or “antigenic determinant” refers to a site on an antigen to which an antibody (e.g., bispecific antibody disclosed herein) binds. Epitopes can be formed both from contiguous amino acids (linear epitope) or noncontiguous amino acids juxtaposed by tertiary folding of a protein (conformational epitopes). Epitopes formed from contiguous amino acids are typically retained on exposure to denaturing solvents, whereas epitopes formed by tertiary folding are typically lost on treatment with denaturing solvents. An epitope typically includes at least 3, and more usually, at least 5 or 8-10 amino acids in a unique spatial conformation. Methods of determining spatial conformation of epitopes include, for example, x-ray crystallography and 2-dimensional nuclear magnetic resonance. See, e.g., Epitope Mapping Protocols in Methods in Molecular Biology, Vol. 66, Glenn E. Morris, Ed (1996).

An “anti-antigen” antibody refers to an antibody that binds specifically to the antigen. For example, an anti-PD-L1 antibody binds specifically to PD-L1, and an anti-CD3ε antibody binds specifically to CD3ε.

An “antigen-binding portion” of an antibody (also called an “antigen-binding fragment”) refers to one or more fragments of an antibody that retain the ability to bind specifically to the antigen bound by the whole antibody.

As used herein, the terms “specific binding,” “selective binding,” “selectively binds,” and “specifically binds,” refer to antibody binding to an epitope on a predetermined antigen. Typically, the antibody (i) binds with an equilibrium dissociation constant (K_{D}) of approximately less than 10⁻⁷ M, such as approximately less than 10⁻⁸ M, 10⁻⁹ M or 10⁻¹⁰ M or even lower when determined by, e.g., surface plasmon resonance (SPR) technology in a BIACORE™ 2000 instrument using the predetermined antigen as the analyte and the antibody as the ligand, or Scatchard analysis of binding of the antibody to antigen positive cells, and (ii) binds to the predetermined antigen with an affinity that is at least two-fold greater than its affinity for binding to a non-specific antigen (e.g., BSA, casein) other than the predetermined antigen or a closely-related antigen.

The term “naturally-occurring” as used herein as applied to an object refers to the fact that an object can be found in nature. For example, a polypeptide or polynucleotide sequence that is present in an organism (including viruses) that can be isolated from a source in nature and which has not been intentionally modified by man in the laboratory is naturally-occurring.

A “polypeptide” refers to a chain comprising at least two consecutively linked amino acid residues, with no upper limit on the length of the chain. One or more amino acid residues in the protein can contain a modification such as, but not limited to, glycosylation, phosphorylation or disulfide bond formation. A “protein” can comprise one or more polypeptides. Unless otherwise specified, the terms “protein” and “polypeptide” can be used interchangeably.

The term “nucleic acid molecule,” as used herein, is intended to include DNA molecules and RNA molecules. A nucleic acid molecule can be single-stranded or double-stranded, and can be cDNA.

“Conservative amino acid substitutions” refer to substitutions of an amino acid residue with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g., threonine, valine, isoleucine) and aromatic side chains (e.g., tyrosine, phenylalanine, tryptophan, histidine). In certain aspects, a predicted nonessential amino acid residue in an antibody is replaced with another amino acid residue from the same side chain family. Methods of identifying nucleotide and amino acid conservative substitutions which do not eliminate antigen binding are well-known in the art (see, e.g., Brummell et at, Biochem. 32: 1180-1187 (1993); Kobayashi et al. Protein Eng. 12(10):879-884 (1999); and Burks et al. Proc. Natl. Acad. Sci. USA 94:412-417 (1997)).

For nucleic acids, the term “substantial homology” indicates that two nucleic acids, or designated sequences thereof, when optimally aligned and compared, are identical, with appropriate nucleotide insertions or deletions, in at least about 80% of the nucleotides, at least about 90% to 95%, or at least about 98% to 99.5% of the nucleotides. Alternatively, substantial homology exists when the segments will hybridize under selective hybridization conditions, to the complement of the strand.

For polypeptides, the term “substantial homology” indicates that two polypeptides, or designated sequences thereof, when optimally aligned and compared, are identical, with appropriate amino acid insertions or deletions, in at least about 80% of the amino acids, at least about 90% to 95%, or at least about 98% to 99.5% of the amino acids.

The percent identity between two sequences is a function of the number of identical positions shared by the sequences (i.e., % homology=# of identical positions/total # of positions×100), taking into account the number of gaps, and the length of each gap, which need to be introduced for optimal alignment of the two sequences. The comparison of sequences and determination of percent identity between two sequences can be accomplished using a mathematical algorithm, e.g., as described in the non-limiting examples below.

The percent identity between two nucleotide sequences can be determined using the GAP program in the GCG software package (available at worldwideweb.gcg.com), using a NWSgapdna.CMP matrix and a gap weight of 40, 50, 60, 70, or 80 and a length weight of 1, 2, 3, 4, 5, or 6. The percent identity between two nucleotide or amino acid sequences can also be determined using the algorithm of E. Meyers and W. Miller (CABIOS, 4: 11-17 (1989)) which has been incorporated into the ALIGN program (version 2.0), using a PAM120 weight residue table, a gap length penalty of 12 and a gap penalty of 4. In addition, the percent identity between two amino acid sequences can be determined using the Needleman and Wunsch (J. Mol. Biol. (48):444-453 (1970)) algorithm which has been incorporated into the GAP program in the GCG software package (available at worldwideweb.gcg.com), using either a Blossum 62 matrix or a PAM250 matrix, and a gap weight of 16, 14, 12, 10, 8, 6, or 4 and a length weight of 1, 2, 3, 4, 5, or 6.

The nucleic acid and protein sequences described herein can further be used as a “query sequence” to perform a search against public databases to, for example, identify related sequences. Such searches can be performed using the NBLAST and)(BLAST programs (version 2.0) of Altschul, et al. (1990) J. Mol. Biol. 215:403-10. BLAST nucleotide searches can be performed with the NBLAST program, score=100, wordlength=12 to obtain nucleotide sequences homologous to the nucleic acid molecules described herein. BLAST protein searches can be performed with the XBLAST program, score=50, wordlength=3 to obtain amino acid sequences homologous to the protein molecules described herein. To obtain gapped alignments for comparison purposes, Gapped BLAST can be utilized as described in Altschul et al., (1997) Nucleic Acids Res. 25(17):3389-3402. When utilizing BLAST and Gapped BLAST programs, the default parameters of the respective programs (e.g.,)(BLAST and NBLAST) can be used. See worldwideweb.ncbi.nlm.nih.gov.

The nucleic acids can be present in whole cells, in a cell lysate, or in a partially purified or substantially pure form. A nucleic acid is “isolated” or “rendered substantially pure” when purified away from other cellular components or other contaminants, e.g., other cellular nucleic acids (e.g., the other parts of the chromosome) or proteins, by standard techniques, including alkaline/SDS treatment, CsCl banding, column chromatography, agarose gel electrophoresis and others well known in the art. See, F. Ausubel, et al., ed. Current Protocols in Molecular Biology, Greene Publishing and Wiley Interscience, New York (1987).

Nucleic acids, e.g., cDNA, can be mutated, in accordance with standard techniques to provide gene sequences. For coding sequences, these mutations, can affect amino acid sequence as desired. In particular, DNA sequences substantially homologous to or derived from native V, D, J, constant, switches and other such sequences described herein are contemplated (where “derived” indicates that a sequence is identical or modified from another sequence).

The term “vector,” as used herein, is intended to refer to a nucleic acid molecule capable of transporting another nucleic acid to which it has been linked. One type of vector is a “plasmid,” which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome. Moreover, certain vectors are capable of directing the expression of genes to which they are operatively linked. Such vectors are referred to herein as “recombinant expression vectors” (or simply, “expression vectors”) In general, expression vectors of utility in recombinant DNA techniques are often in the form of plasmids. In the present specification, “plasmid” and “vector” can be used interchangeably as the plasmid is the most commonly used form of vector. However, also included are other forms of expression vectors, such as viral vectors (e.g., replication defective retroviruses, adenoviruses and adeno-associated viruses), which serve equivalent functions.

The term “recombinant host cell” (or simply “host cell”), as used herein, is intended to refer to a cell that comprises a nucleic acid that is not naturally present in the cell, and can be a cell into which a recombinant expression vector has been introduced. It should be understood that such terms are intended to refer not only to the particular subject cell but to the progeny of such a cell. Because certain modifications can occur in succeeding generations due to either mutation or environmental influences, such progeny cannot, in fact, be identical to the parent cell, but are still included within the scope of the term “host cell” as used herein.

As used herein, the term “linked” refers to the association of two or more molecules. The linkage can be covalent or non-covalent. The linkage also can be genetic (i.e., recombinantly fused). Such linkages can be achieved using a wide variety of art recognized techniques, such as chemical conjugation and recombinant protein production.

A “cancer” refers a broad group of various diseases characterized by the uncontrolled growth of abnormal cells in the body. Unregulated cell division and growth results in the formation of malignant tumors that invade neighboring tissues and can also metastasize to distant parts of the body through the lymphatic system or bloodstream. “Cancer” as used herein refers to primary, metastatic and recurrent cancers.

The term “fusion protein” refers to proteins created through the joining of two or more genes that originally coded for separate proteins. Translation of this fusion gene results in a single polypeptide or multiple polypeptides with functional properties derived from each of the original proteins. In some aspects, the two or more genes can comprise a substitution, a deletion, and/or an addition in its nucleotide sequence.

An “Fc receptor” or “FcR” is a receptor that binds to the Fc region of an immunoglobulin. FcRs that bind to an IgG antibody comprise receptors of the FcγR family, including allelic variants and alternatively spliced forms of these receptors. The FcγR family consists of three activating (FcγRI, FcγRIII, and FcγRIV in mice; FcγRIA, FcγRIIA, and FcγRIIIA in humans) and one inhibitory (FcγRIIB) receptor. Various properties of human FcγRs are known in the art. The majority of innate effector cell types coexpress one or more activating FcγR and the inhibitory FcγRIIB, whereas natural killer (NK) cells selectively express one activating Fc receptor (FcγRIII in mice and FcγRIIIA in humans) but not the inhibitory FcγRIIB in mice and humans. Human IgG1 binds to most human Fc receptors and is considered equivalent to murine IgG2a with respect to the types of activating Fc receptors that it binds to.

An “Fc region” (fragment crystallizable region) or “Fc domain” or “Fc” refers to the C-terminal region of the heavy chain of an antibody that mediates the binding of the immunoglobulin to host tissues or factors, including binding to Fc receptors located on various cells of the immune system (e.g., effector cells) or to the first component (Clq) of the classical complement system. Thus, an Fc region comprises the constant region of an antibody excluding the first constant region immunoglobulin domain (e.g., CH1 or CL). In IgG, IgA and IgD antibody isotypes, the Fc region comprises two identical protein fragments, derived from the second (CH2) and third (CH3) constant domains of the antibody's two heavy chains; IgM and IgE Fc regions comprise three heavy chain constant domains (CH domains 2-4) in each polypeptide chain. For IgG, the Fc region comprises immunoglobulin domains CH2 and CH3 and the hinge between CH1 and CH2 domains. Although the definition of the boundaries of the Fc region of an immunoglobulin heavy chain might vary, as defined herein, the human IgG heavy chain Fc region is defined to stretch from an amino acid residue D221 for IgG1, V222 for IgG2, L221 for IgG3 and P224 for IgG4 to the carboxy-terminus of the heavy chain, wherein the numbering is according to the EU index as in Kabat. The CH2 domain of a human IgG Fc region extends from amino acid 237 to amino acid 340, and the CH3 domain is positioned on C-terminal side of a CH2 domain in an Fc region, i.e., it extends from amino acid 341 to amino acid 447 or 446 (if the C-terminal lysine residue is absent) or 445 (if the C-terminal glycine and lysine residues are absent) of an IgG. As used herein, the Fc region can be a native sequence Fc, including any allotypic variant, or a variant Fc (e.g., a non-naturally occurring Fc). Fc can also refer to this region in isolation or in the context of an Fc-comprising protein polypeptide such as a “binding protein comprising an Fc region,” also referred to as an “Fc fusion protein” (e.g., an antibody or immunoadhesion).

A “native sequence Fc region” or “native sequence Fc” comprises an amino acid sequence that is identical to the amino acid sequence of an Fc region found in nature. Native sequence human Fc regions include a native sequence human IgG1 Fc region; native sequence human IgG2 Fc region; native sequence human IgG3 Fc region; and native sequence human IgG4 Fc region as well as naturally occurring variants thereof. Native sequence Fc include the various allotypes of Fcs (see, e.g., Jefferis et al. (2009) mAbs 1: 1).

Additionally, an Fc (native or variant) of the present invention can be in the form of having native sugar chains, increased sugar chains, or decreased sugar chains compared to the native form, or may be in a deglycosylated form. The immunoglobulin Fc sugar chains can be modified by conventional methods such as a chemical method, an enzymatic method, and a genetic engineering method using a microorganism. The removal of sugar chains from an Fc fragment results in a sharp decrease in binding affinity to the C1q part of the first complement component C1, and a decrease or loss of ADCC or CDC, thereby not inducing any unnecessary immune responses in vivo. In this regard, an immunoglobulin Fc region in a deglycosylated or aglycosylated form may be more suitable to the object of the present invention as a drug carrier. As used herein, the term “deglycosylation” refers to an Fc region in which sugars are removed enzymatically from an Fc fragment. Additionally, the term “aglycosylation” means that an Fc fragment is produced in an unglycosylated form by a prokaryote, and preferably in E. coli.

As used herein, the term “immune response” refers to a biological response within a vertebrate against foreign agents, which response protects the organism against these agents and diseases caused by them. An immune response is mediated by the action of a cell of the immune system (e.g., a T lymphocyte, B lymphocyte, natural killer (NK) cell, macrophage, eosinophil, mast cell, dendritic cell or neutrophil) and soluble macromolecules produced by any of these cells or the liver (including antibodies, cytokines, and complement) that results in selective targeting, binding to, damage to, destruction of, and/or elimination from the vertebrate's body of invading pathogens, cells or tissues infected with pathogens, cancerous or other abnormal cells, or, in cases of autoimmunity or pathological inflammation, normal human cells or tissues. An immune reaction includes, e.g., activation or inhibition of a T cell, e.g., an effector T cell or a Th cell, such as a CD4⁺ or CD8⁺ T cell, or the inhibition of a Treg cell.

An “immunomodulator” or “immunoregulator” refers to an agent, e.g., a component of a signaling pathway, that can be involved in modulating, regulating, or modifying an immune response. “Modulating,” “regulating,” or “modifying” an immune response refers to any alteration in a cell of the immune system or in the activity of such cell (e.g., an effector T cell). Such modulation includes stimulation or suppression of the immune system which can be manifested by an increase or decrease in the number of various cell types, an increase or decrease in the activity of these cells, or any other changes which can occur within the immune system. Both inhibitory and stimulatory immunomodulators have been identified, some of which can have enhanced function in a tumor microenvironment. In preferred aspects, the immunomodulator is located on the surface of a T cell. An “immunomodulatory target” or “immunoregulatory target” is an immunomodulator that is targeted for binding by, and whose activity is altered by the binding of, a substance, agent, moiety, compound or molecule. Immunomodulatory targets include, for example, receptors on the surface of a cell (“immunomodulatory receptors”) and receptor ligands (“immunomodulatory ligands”).

The term “immunotherapy” refers to the treatment of a subject afflicted with, or at risk of contracting or suffering a recurrence of, a disease by a method comprising inducing, enhancing, suppressing or otherwise modifying an immune response. “Treatment” or “therapy” of a subject refers to any type of intervention or process performed on, or the administration of an active agent to, the subject with the objective of reversing, alleviating, ameliorating, inhibiting, slowing down or preventing the onset, progression, development, severity or recurrence of a symptom, complication or condition, or biochemical indicia associated with a disease.

“Immunostimulating therapy” or “immunostimulatory therapy” refers to a therapy that results in increasing (inducing or enhancing) an immune response in a subject for, e.g., treating cancer.

The term “effector T cells” (Teff) refers to T cells (e.g., CD4⁺ and CD8⁺ T cells) with cytolytic activities as well as T helper (Th) cells, which secrete cytokines and activate and direct other immune cells, but does not include regulatory T cells (Treg cells). Combination of an IL-7 protein and an immune checkpoint inhibitor (e.g., an anti-PD-1 antibody) activate and/or increase the frequency of Teff cells, e.g., CD4⁺ and CD8⁺ T cells, in a tumor or blood of a subject.

As used herein, the term “regulatory T cells” (Tregs) refer to a population of T cells with the ability to reduce or suppress the induction and proliferation of effector T cells, and thereby, modulate an immune response. In some aspects, Tregs can suppress an immune response by secreting anti-inflammatory cytokines, such as IL-10, TGF-β, and IL-35, which can interfere with the activation and differentiation of naïve T cells into effector T cells. In some aspects, Tregs can also produce cytolytic molecules, such as Granzyme B, which can induce the apoptosis of effector T cells. In some aspects, the regulatory T cells are natural regulatory T cells (nTregs) (i.e., developed within the thymus). In some aspects, the regulatory T cells are induced regulatory T cells (iTregs) (i.e., naïve T cells that differentiate into Tregs in the peripheral tissue upon exposure to certain stimuli). Methods for identifying Tregs are known in the art. For example, Tregs express certain phenotypic markers (e.g., CD25, Foxp3, or CD39) that can be measured using flow cytometry. See, e.g., International Publication No. WO 2017/062035 A1; Gu J., et al., Cell Mol Immunol 14(6): 521-528 (2017). In some aspects, the Tregs are CD45RA⁻CD39⁺ T cells.

As used herein, the term “tumor infiltrating lymphocytes” or “TILs” refers to lymphocytes (e.g., effector T cells) that have migrated from the periphery (e.g., from the blood) into a tumor. In some aspects, the tumor infiltrating lymphocytes are CD4+ TILs. In other aspects, the tumor infiltrating lymphocytes are CD8+ TILs.

An increased ability to stimulate an immune response or the immune system, can result from an enhanced agonist activity of T cell costimulatory receptors and/or an enhanced antagonist activity of inhibitory receptors. An increased ability to stimulate an immune response or the immune system can be reflected by a fold increase of the EC50 or maximal level of activity in an assay that measures an immune response, e.g., an assay that measures changes in cytokine or chemokine release, cytolytic activity (determined directly on target cells or indirectly via detecting CD107a or granzymes) and proliferation. The ability to stimulate an immune response or the immune system activity can be enhanced by at least 10%, 30%, 50%, 75%, 2 fold, 3 fold, 5 fold or more.

As used herein, the term “interleukin-7” or “IL-7” refers to IL-7 polypeptides and derivatives and analogs thereof having substantial amino acid sequence identity to wild-type mature mammalian IL-7s and substantially equivalent biological activity, e.g., in standard bioassays or assays of IL-7 receptor binding affinity. For example, IL-7 refers to an amino acid sequence of a recombinant or non-recombinant polypeptide having an amino acid sequence of: i) a native or naturally-occurring allelic variant of an IL-7 polypeptide, ii) a biologically active fragment of an IL-7 polypeptide, iii) a biologically active polypeptide analog of an IL-7 polypeptide, or iv) a biologically active variant of an IL-7 polypeptide. IL-7 polypeptides of the invention can be obtained from any species, e.g., human, cow or sheep. IL-7 nucleic acid and amino acid sequences are well known in the art. For example, the human IL-7 amino acid sequence has a Genbank accession number of P13232 (SEQ ID NO: 1); the mouse IL-7 amino acid sequence has a Genbank accession number of P10168 (SEQ ID NO: 3); the rat IL-7 amino acid sequence has a Genbank accession number of P56478 (SEQ ID NO: 2); the monkey IL-7 amino acid sequence has a Genbank accession number of NP_001279008 (SEQ ID NO: 4); the cow IL-7 amino acid sequence has a Genbank accession number of P26895 (SEQ ID NO: 5); and the sheep IL-7 amino acid sequence has a Genbank accession number of Q28540 (SEQ ID NO: 6). In some aspects, an IL-7 polypeptide of the present disclosure is a variant of an IL-7 protein.

A “variant” of an IL-7 protein is defined as an amino acid sequence that is altered by one or more amino acids. The variant can have “conservative” changes, wherein a substituted amino acid has similar structural or chemical properties, e.g., replacement of leucine with isoleucine. More rarely, a variant can have “nonconservative” changes, e.g., replacement of a glycine with a tryptophan. Similar minor variations can also include amino acid deletions or insertions, or both. Guidance in determining which and how many amino acid residues may be substituted, inserted or deleted without abolishing biological activity can be found using computer programs well known in the art, for example software for molecular modeling or for producing alignments. The variant IL-7 proteins included within the invention include IL-7 proteins that retain IL-7 activity. IL-7 polypeptides which also include additions, substitutions or deletions are also included within the invention as long as the proteins retain substantially equivalent biological IL-7 activity. For example, truncations of IL-7 which retain comparable biological activity as the full length form of the IL-7 protein are included within the invention. The activity of the IL-7 protein can be measured using in vitro cellular proliferation assays such as described in Example 6 below. The activity of IL-7 variants of the invention maintain biological activity of at least 10%, 20%, 40%, 60%, but more preferably 80%, 90%, 95% and even more preferably 99% as compared to wild type IL-7.

Variant IL-7 proteins also include polypeptides that have at least about 70%, 75%, 80%, 85%, 90%, 92%, 95%, 96%, 97%, 98%, 99%, or more sequence identity with wild-type IL-7. To determine the percent identity of two amino acid sequences or of two nucleic acids, the sequences are aligned for optimal comparison purposes (e.g., gaps can be introduced in the sequence of a first amino acid or nucleic acid sequence for optimal alignment with a second amino acid or nucleic acid sequence). The percent identity between the two sequences is a function of the number of identical positions shared by the sequences (i.e., % homology=# of identical positions/total # of positions·times·100). The determination of percent homology between two sequences can be accomplished using a mathematical algorithm. A preferred, non-limiting example of a mathematical algorithm utilized for the comparison of two sequences is the algorithm of Karlin and Altschul (1990) Proc. Natl. Acad. Sci. USA 87:2264-68, modified as in Karlin and Altschul (1993) Proc. Natl. Acad. Sci. USA 90:5873-77. Such an algorithm is incorporated into the NBLAST and XBLAST programs of Altschul, et al., (1990) J. Mol. Biol. 215:403-10. BLAST nucleotide searches can be performed with the NBLAST program, score=100, wordlength=12. BLAST protein searches can be performed with the) (BLAST program, score=50, wordlength=3. To obtain gapped alignments for comparison purposes, Gapped BLAST can be utilized as described in Altschul et al., (1997) Nucleic Acids Research 25(17):3389-3402. When utilizing BLAST and Gapped BLAST programs, the default parameters of the respective programs (e.g.,)(BLAST and NBLAST) can be used.

As used herein, the term “Programmed Death-1 (PD-1)” refers to an immunoinhibitory receptor belonging to the CD28 family. PD-1 is expressed predominantly on previously activated T cells in vivo, and binds to two ligands, PD-L1 and PD-L2. The term “PD-1” as used herein includes human PD-1 (hPD-1), variants, isoforms, and species homologs of hPD-1, and analogs having at least one common epitope with hPD-1. The complete hPD-1 sequence can be found under GenBank Accession No. U64863.

As used herein, the term “Programmed Death Ligand-1 (PD-L1)” refers to one of two cell surface glycoprotein ligands for PD-1 (the other being PD-L2) that downregulate T cell activation and cytokine secretion upon binding to PD-1. The term “PD-L1” as used herein includes human PD-L1 (hPD-L1), variants, isoforms, and species homologs of hPD-L1, and analogs having at least one common epitope with hPD-L1. The complete hPD-L1 sequence can be found under GenBank Accession No. Q9NZQ7.

As used herein, the term “guanylate cyclase C (GC-C)” refers to a membrane-bound enzyme present primarily within the intestinal lumen of a mammal (e.g., human) and contributes to the maintenance of normal physiological functioning of the gastrointestinal tract. Ligands for GC-C are known in the art. See Waldman, S. A., et al., Gut 67(8): 1543-1552 (2018). In some aspects, GC-C serves as a receptor for heat-stable toxin (ST) peptides secreted by enteric bacteria. In some aspects, GC-C serves as a receptor for guanylin and uroguanylin peptides, which are produced naturally within the gastrointestinal tract. GC-C is also known as guanylate cyclase 2C, intestinal guanylate cyclase, guanylate cyclase-C receptor, or heat-stable enterotoxin receptor (hSTAR). The full-length sequence of human GC-C is known and can be found under GenBank Accession No. P25092.2.

A “subject” includes any human or nonhuman animal. The term “nonhuman animal” includes, but is not limited to, vertebrates such as nonhuman primates, sheep, dogs, and rodents such as mice, rats and guinea pigs. In some aspects, the subject is a human. The terms “subject” and “patient” are used interchangeably herein.

The term “therapeutically effective amount” or “therapeutically effective dosage” refers to an amount of an agent that provides the desired biological, therapeutic, and/or prophylactic result. That result can be reduction, amelioration, palliation, lessening, delaying, and/or alleviation of one or more of the signs, symptoms, or causes of a disease, or any other desired alteration of a biological system. In reference to solid tumors, an effective amount comprises an amount sufficient to cause a tumor to shrink and/or to decrease the growth rate of the tumor (such as to suppress tumor growth) or to prevent or delay other unwanted cell proliferation. In some aspects, an effective amount is an amount sufficient to delay tumor development. In some aspects, an effective amount is an amount sufficient to prevent or delay tumor recurrence. An effective amount can be administered in one or more administrations. The effective amount of the drug or composition can: (i) reduce the number of cancer cells; (ii) reduce tumor size; (iii) inhibit, retard, slow to some extent and can stop cancer cell infiltration into peripheral organs; (iv) inhibit (i.e., slow to some extent and can stop tumor metastasis; (v) inhibit tumor growth; (vi) prevent or delay occurrence and/or recurrence of tumor; and/or (vii) relieve to some extent one or more of the symptoms associated with the cancer. In some aspects, a “therapeutically effective amount” is the amount of IL-7 protein and the amount of a bispecific antibody, in combination, clinically proven to affect a significant decrease in cancer or slowing of progression (regression) of cancer, such as an advanced solid tumor. The ability of a therapeutic agent to promote disease regression can be evaluated using a variety of methods known to the skilled practitioner, such as in human subjects during clinical trials, in animal model systems predictive of efficacy in humans, or by assaying the activity of the agent in in vitro assays.

The term “dosing frequency” refers to the number of times a therapeutic agent (e.g., an IL-7 protein or bispecific antibody) is administered to a subject within a specific time period. Dosing frequency can be indicated as the number of doses per a given time, for example, once per day, once a week, or once in two weeks. As used herein, “dosing frequency” is applicable where a subject receives multiple (or repeated) administrations of a therapeutic agent.

As used herein, the term “standard of care” refers to a treatment that is accepted by medical experts as a proper treatment for a certain type of disease and that is widely used by healthcare professionals. The term can be used interchangeable with any of the following terms: “best practice,” “standard medical care,” and “standard therapy.”

As used herein, the term “drug” refers to any bioactive agent (e.g., an IL-7 protein or a bispecific antibody disclosed herein) intended for administration to a human or non-human mammal to prevent or treat a disease or other undesirable condition. Drugs include hormones, growth factors, proteins, peptides and other compounds. In some aspects, a drug disclosed herein is an anti-cancer agent.

By way of example, an “anti-cancer agent” promotes cancer regression in a subject or prevents further tumor growth. In certain aspects, a therapeutically effective amount of the drug promotes cancer regression to the point of eliminating the cancer. “Promoting cancer regression” means that administering an effective amount of the drug, alone or in combination with an anti-neoplastic agent, results in a reduction in tumor growth or size, necrosis of the tumor, a decrease in severity of at least one disease symptom, an increase in frequency and duration of disease symptom-free periods, or a prevention of impairment or disability due to the disease affliction. In addition, the terms “effective” and “effectiveness” with regard to a treatment includes both pharmacological effectiveness and physiological safety. Pharmacological effectiveness refers to the ability of the drug to promote cancer regression in the patient. Physiological safety refers to the level of toxicity, or other adverse physiological effects at the cellular, organ and/or organism level (adverse effects) resulting from administration of the drug.

By way of example, for the treatment of tumors, a therapeutically effective amount of an anti-cancer agent can inhibit cell growth or tumor growth by at least about 10%, at least about 20%, by at least about 40%, by at least about 60%, or by at least about 80% relative to untreated subjects or, in certain aspects, relative to patients treated with a standard-of-care therapy. In other aspects of the invention, tumor regression can be observed and continue for a period of at least about 20 days, at least about 40 days, or at least about 60 days. Notwithstanding these ultimate measurements of therapeutic effectiveness, evaluation of immunotherapeutic drugs must also make allowance for “immune-related” response patterns.

As used herein, the term “immune checkpoint inhibitor” refers to molecules that totally or partially reduce, inhibit, interfere with or modulate one or more immune checkpoint proteins. Immune checkpoint proteins regulate T-cell activation or function. Numerous checkpoint proteins are known, such as CTLA-4 and its ligands CD80 and CD86; and PD-1 with its ligands PD-L1 and PD-L2. Pardoll, D. M., Nat Rev Cancer 12(4):252-64 (2012). These proteins are responsible for co-stimulatory or inhibitory interactions of T-cell responses. Immune checkpoint proteins regulate and maintain self-tolerance and the duration and amplitude of physiological immune responses. Immune checkpoint inhibitors include antibodies or are derived from antibodies.

The term “reference,” as used herein, refers to a corresponding subject (e.g., a cancer subject) who did not receive a combination of an IL-7 protein and a bispecific antibody, e.g., a subject who received an IL-7 protein alone or a bispecific antibody alone. In some aspects, the reference subject received neither an IL-7 protein nor a bispecific antibody. The term “reference” can also refer to a same cancer subject but prior to the administration of a combination of an IL-7 protein and a bispecific antibody. In certain aspects, the term “reference” refers to an average of a population of subjects (e.g., cancer subjects).

As used herein, the terms “ug” and “uM” are used interchangeably with “μg” and “μM,” respectively.

Various aspects described herein are described in further detail in the following subsections.

### II. Methods of the Disclosure

The present disclosure is directed to a method for treating a tumor (or a cancer) in a subject in need thereof, comprising administering to the subject an effective amount of an interleukin-7 (IL-7) protein in combination with an effective amount of a bispecific antibody. In some aspects, a bispecific antibody that is useful for the present disclosure can bind to a tumor antigen and an antigen expressed on an immune cell. In certain aspects, administering an IL-7 protein in combination with a bispecific antibody can increase the anti-cancer effects of the bispecific antibody. In some aspects, administering an IL-7 protein in combination with a bispecific antibody improves one or more symptoms associated with a tumor (or a cancer) as described herein. Additional disclosure relating to exemplary bispecific antibodies that can be used with the present disclosure are also provided elsewhere herein.

In some aspects, a combination of IL-7 protein and a bispecific antibody disclosed herein inhibits and/or reduces tumor growth (e.g., tumor volume or weight) in a subject. In some aspects, the tumor growth is reduced by at least about 5%, at least about 10%, at least about 20%, at least about 30%, at least about 40%, at least about 50%, at least about 60%, at least about 70%, at least about 80%, at least about 90%, or about 100% compared to a reference (e.g., tumor volume in a corresponding subject after administration of IL-7 protein alone or bispecific antibody alone).

In some aspects, a combination of IL-7 protein and bispecific antibody does not cause toxicity when administered to a subject. In certain aspects, the lack of toxicity can correspond to minimal loss of body weight loss. Accordingly, in some aspects, a combination of an IL-7 protein and a bispecific antibody does not cause loss of body weight in a subject after the administration of the IL-7 protein and bispecific antibody, compared to a reference body weight (e.g., body weight of the subject prior to administration and/or body weight of a subject after administration of either the IL-7 protein or the bispecific antibody alone). In certain aspects, the body weight of a subject that received an IL-7 protein in combination with a bispecific antibody does not decrease by more than about 1%, about 2%, about 3%, about 4%, about 5%, about 6%, about 7%, about 8%, about 9%, about 10%, about 15%, or about 20%, compared to the reference body weight (e.g., body weight of the subject prior to administration).

In some aspects, a combination of IL-7 protein and a bispecific antibody disclosed herein can increase the absolute lymphocyte count in a subject when administered to the subject. In certain aspects, the absolute lymphocyte count is increased by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 100% or more, compared to a reference (e.g., value in a corresponding subject after administration of IL-7 protein alone or bispecific antibody alone).

In some aspects, a combination of IL-7 protein and a bispecific antibody disclosed herein can promote and/or enhance an immune response against a tumor antigen. In some aspects, administering a combination of IL-7 protein and a bispecific antibody increases the number and/or percentage of tumor-infiltrating lymphocytes (TILs) (e.g., CD4⁺ or CD8⁺) in a tumor of a subject. In some aspects, the number and/or percentage of TILs is increased by at least about 10%, at least about 20%, at least about 30%, at least about 40%, at least about 50%, at least about 60%, at least about 70%, at least about 80%, at least about 90%, at least about 100%, at least about 125%, at least about 150%, at least about 200%, at least about 250%, or at least about 300% after the administration compared to a reference (e.g., value in a corresponding subject after administration of IL-7 protein alone or bispecific antibody alone).

In some aspects, administering a combination of IL-7 protein and a bispecific antibody reduces the number and/or percentage of regulatory T cells (Tregs) in a tumor of a subject. In some aspects, the regulatory T cells are CD4⁺ regulatory T cells. In some aspects, the regulatory T cells are Foxp3⁺. In certain aspects, the number and/or percentage of regulatory T cells in a tumor is decreased by at least about 5%, at least about 10%, at least about 20%, at least about 30%, at least about 40%, at least about 50%, at least about 60%, at least about 70%, at least about 80%, at least about 90%, or about 100% compared to a reference (e.g., value in a corresponding subject after administration of IL-7 protein alone or bispecific antibody alone).

In some aspects, administering a combination of an IL-7 protein and a bispecific antibody increases the ratio of CD8⁺ TILs to Tregs in a tumor of a subject. In certain aspects, the ratio of CD8⁺ TILs to Tregs is increased by at least about 5%, at least about 10%, at least about 20%, at least about 30%, at least about 40%, at least about 50%, at least about 60%, at least about 70%, at least about 80%, at least about 90%, at least about 100%, at least about 125%, at least about 150%, at least about 200%, at least about 250%, or at least about 300% after the administration compared to a reference (e.g., value in a corresponding subject after administration of IL-7 protein alone or bispecific antibody alone).

In some aspects, administering a combination of an IL-7 protein and an bispecific antibody reduces the expression of an immune checkpoint inhibitor molecule (e.g., PD-1) on TILs in a subject. In some aspects, administering a combination of an IL-7 protein and a bispecific antibody increases the expression of markers associated with effector (e.g., anti-tumor) activity on TILs in a subject. Non-limiting examples of markers associated with effector activity includes CD69, CD25, and granzyme B.

As described supra, many cancer patients are lymphopenic, as many of the available standard of care cancer treatments (e.g., chemotherapy and radiation therapy) are known to cause lymphopenia. Accordingly, methods disclosed herein can also be used to treat a cancer in a lymphopenic subject.

As used herein, the term “lymphopenic subject” refers to a subject with lymphopenia. As used herein, the terms “lymphopenia” and “lymphocytopenia” are used interchangeably and refer to a condition characterized by abnormally low number of circulating immune cells (e.g., lymphocytes). Peripheral circulation of all types of lymphocytes or subpopulations of lymphocytes (e.g., CD4⁺ T cells) can be depleted or abnormally low in a patient suffering from lymphopenia. See, e.g., Lymphopenia Description, The Merck Manual (18th Edition, 2006, Merck & Co.). In some aspects, compared to a normal subject (e.g., healthy individual), a lymphopenic subject has reduced number of T-lymphocytes (“T-lymphopenia”), B-lymphocytes (“B-lymphopenia”), and/or NK cells (“NK lymphopenia”).

Quantitatively, lymphopenia can be described by various cutoffs. In some aspects, a lymphopenic subject has a circulating blood total lymphocyte count that is less than by at least about 5%, at least about 10%, at least about 20%, at least about 30%, at least about 40%, at least about 50%, at least about 60%, at least about 70%, at least about 80%, at least about 90%, or at least about 100% compared to a circulating blood total lymphocyte count in a corresponding subject who does not exhibit a lymphopenia. In some aspects, a subject has lymphopenia if the subject has a circulating blood total lymphocyte count of less than about 1,500 lymphocytes/μL, less than about 1,000 lymphocytes/μL, less than about 800 lymphocytes/μL, less than about 500 lymphocytes/μL, or less than about 200 lymphocytes/μL.

Lymphocytopenia has a wide range of possible causes. In some aspects, a lymphopenia is caused by or associated with a tumor. In some aspects, a lymphopenia is caused by or associated with a previous therapy for a tumor (e.g., chemotherapy or radiation therapy). In some aspects, a lymphopenia is caused by or associated with an infection, including viral (e.g., HIV or hepatitis infection), bacterial (e.g., active tuberculosis infection), and fungal infections; chronic failure of the right ventricle of the heart, Hodgkin's disease and cancers of the lymphatic system, leukemia, a leak or rupture in the thoracic duct, side effects of prescription medications including anticancer agents, antiviral agents, and glucocorticoids, malnutrition resulting from diets that are low in protein, radiation therapy, uremia, autoimmune disorders, immune deficiency syndromes, high stress levels, and trauma.

In some aspects, a lymphopenia is idiopathic (i.e., has unknown etiology). Non-limiting examples of idiopathic lymphopenia include idiopathic CD4 positive T-lymphocytopenia (ICL), acute radiation syndrome (ARS), or a combination thereof.

Non-limiting examples of cancers (or tumors) that can be treated with methods disclosed herein include squamous cell carcinoma, small-cell lung cancer (SCLC), non-small cell lung cancer, squamous non-small cell lung cancer (NSCLC), nonsquamous NSCLC, gastrointestinal cancer, renal cancer (e.g., clear cell carcinoma), ovarian cancer, liver cancer (e.g., hepatocellular carcinoma), colorectal cancer, endometrial cancer, kidney cancer (e.g., renal cell carcinoma (RCC)), prostate cancer (e.g., hormone refractory prostate adenocarcinoma), thyroid cancer, pancreatic cancer, cervical cancer, stomach cancer, bladder cancer, hepatoma, breast cancer, colon carcinoma, and head and neck cancer (or carcinoma), gastric cancer, germ cell tumor, pediatric sarcoma, sinonasal natural killer, melanoma (e.g., metastatic malignant melanoma, such as cutaneous or intraocular malignant melanoma), bone cancer, skin cancer, uterine cancer, cancer of the anal region, testicular cancer, carcinoma of the fallopian tubes, carcinoma of the endometrium, carcinoma of the cervix, carcinoma of the vagina, carcinoma of the vulva, cancer of the esophagus (e.g., gastroesophageal junction cancer), cancer of the small intestine, cancer of the endocrine system, cancer of the parathyroid gland, cancer of the adrenal gland, sarcoma of soft tissue, cancer of the urethra, cancer of the penis, solid tumors of childhood, cancer of the ureter, carcinoma of the renal pelvis, tumor angiogenesis, pituitary adenoma, Kaposi's sarcoma, epidermoid cancer, squamous cell cancer, T-cell lymphoma, environmentally-induced cancers including those induced by asbestos, virus-related cancers or cancers of viral origin (e.g., human papilloma virus (HPV-related or -originating tumors)), and hematologic malignancies derived from either of the two major blood cell lineages, i.e., the myeloid cell line (which produces granulocytes, erythrocytes, thrombocytes, macrophages and mast cells) or lymphoid cell line (which produces B, T, NK and plasma cells), such as all types of leukemias, lymphomas, and myelomas, e.g., acute, chronic, lymphocytic and/or myelogenous leukemias, such as acute leukemia (ALL), acute myelogenous leukemia (AML), chronic lymphocytic leukemia (CLL), and chronic myelogenous leukemia (CML), undifferentiated AML (MO), myeloblastic leukemia (M1), myeloblastic leukemia (M2; with cell maturation), promyelocytic leukemia (M3 or M3 variant [M3V]), myelomonocytic leukemia (M4 or M4 variant with eosinophilia [M4E]), monocytic leukemia (M5), erythroleukemia (M6), megakaryoblastic leukemia (M7), isolated granulocytic sarcoma, and chloroma; lymphomas, such as Hodgkin's lymphoma (HL), non-Hodgkin's lymphoma (NHL), B cell hematologic malignancy, e.g., B-cell lymphomas, T-cell lymphomas, lymphoplasmacytoid lymphoma, monocytoid B-cell lymphoma, mucosa-associated lymphoid tissue (MALT) lymphoma, anaplastic (e.g., Ki1⁺) large-cell lymphoma, adult T-cell lymphoma/leukemia, mantle cell lymphoma, angio immunoblastic T-cell lymphoma, angiocentric lymphoma, intestinal T-cell lymphoma, primary mediastinal B-cell lymphoma, precursor T-lymphoblastic lymphoma, T-lymphoblastic; and lymphoma/leukaemia (T-Lbly/T-ALL), peripheral T-cell lymphoma, lymphoblastic lymphoma, post-transplantation lymphoproliferative disorder, true histiocytic lymphoma, primary effusion lymphoma, B cell lymphoma, lymphoblastic lymphoma (LBL), hematopoietic tumors of lymphoid lineage, acute lymphoblastic leukemia, diffuse large B-cell lymphoma, Burkitt's lymphoma, follicular lymphoma, diffuse histiocytic lymphoma (DHL), immunoblastic large cell lymphoma, precursor B-lymphoblastic lymphoma, cutaneous T-cell lymphoma (CTLC) (also called mycosis fungoides or Sezary syndrome), and lymphoplasmacytoid lymphoma (LPL) with Waldenstrom's macroglobulinemia; myelomas, such as IgG myeloma, light chain myeloma, nonsecretory myeloma, smoldering myeloma (also called indolent myeloma), solitary plasmocytoma, and multiple myelomas, chronic lymphocytic leukemia (CLL), hairy cell lymphoma; hematopoietic tumors of myeloid lineage, tumors of mesenchymal origin, including fibrosarcoma and rhabdomyoscarcoma; seminoma, teratocarcinoma, tumors of mesenchymal origin, including fibrosarcoma, rhabdomyoscaroma, and osteosarcoma; and other tumors, including melanoma, xeroderma pigmentosum, keratoacanthoma, seminoma, thyroid follicular cancer and teratocarcinoma, hematopoietic tumors of lymphoid lineage, for example T-cell and B-cell tumors, including but not limited to T-cell disorders such as T-prolymphocytic leukemia (T-PLL), including of the small cell and cerebriform cell type; large granular lymphocyte leukemia (LGL) of the T-cell type; a/d T-NHL hepatosplenic lymphoma; peripheral/post-thymic T cell lymphoma (pleomorphic and immunoblastic subtypes); angiocentric (nasal) T-cell lymphoma; cancer of the head or neck, renal cancer, rectal cancer, cancer of the thyroid gland; acute myeloid lymphoma, and any combinations thereof.

In some aspects, a cancer (or tumor) that can be treated comprises a breast cancer, head and neck cancer, uterine cancer, brain cancer, skin cancer, renal cancer, lung cancer, colorectal cancer, prostate cancer, liver cancer, bladder cancer, kidney cancer, pancreatic cancer, thyroid cancer, esophageal cancer, eye cancer, stomach (gastric) cancer, gastrointestinal cancer, carcinoma, sarcoma, leukemia, lymphoma, myeloma, or a combination thereof. In certain aspects, a cancer (or tumor) that can be treated with the present methods is breast cancer. In some aspects, breast cancer is a triple negative breast cancer (TNBC). In some aspects, a cancer (or tumor) that can be treated is a brain cancer. In certain aspects, brain cancer is a glioblastoma. In some aspects, a cancer (or tumor) that can be treated with the present methods is skin cancer. In some aspects, skin cancer is a basal cell carcinoma (BCC), cutaneous squamous cell carcinoma (cSCC), melanoma, Merkel cell carcinoma (MCC), or a combination thereof In certain aspects, a head and neck cancer is a head and neck squamous cell carcinoma. In further aspects, a lung cancer is a small cell lung cancer (SCLC). In some aspects, an esophageal cancer is gastroesophageal junction cancer. In certain aspects, a kidney cancer is renal cell carcinoma. In some aspects, a liver cancer is hepatocellular carcinoma.

In some aspects, the methods described herein can also be used for treatment of metastatic cancers, unresectable, refractory cancers (e.g., cancers refractory to previous cancer therapy, e.g., immunotherapy, e.g., with a blocking anti-PD-1 antibody), and/or recurrent cancers. In certain aspects, the previous cancer therapy comprises a chemotherapy. In some aspects, the chemotherapy comprises a platinum-based therapy. In some aspects, the platinum-based therapy comprises a platinum-based antineoplastic selected from the group consisting of cisplatin, carboplatin, oxaliplatin, nedaplatin, triplatin tetranitrate, phenanthriplatin, picoplatin, satraplatin, and any combination thereof. In certain aspects, the platinum-based therapy comprises cisplatin. In further aspects, the platinum-based therapy comprises carboplatin.

In some aspects, a subject to be treated with the methods disclosed herein has received one, two, three, four, five or more prior cancer treatments. In other aspects, the subject is treatment-naïve (i.e., has never received a prior cancer treatment). In some aspects, the subject has progressed on other cancer treatments. In certain aspects, the prior cancer treatment comprised an immunotherapy (e.g., with an anti-PD-1 antibody). In other aspects, the prior cancer treatment comprised a chemotherapy. In some aspects, the tumor has reoccurred. In some aspects, the tumor is metastatic. In other aspects, the tumor is not metastatic.

In some aspects, methods disclosed herein effectively increases the duration of survival of a subject in need thereof (e.g., afflicted with a tumor). For example, in some aspects, duration of survival of the subject is increased by at least about 1 month, at least about 2 months, at least about 3 months, at least about 4 months, at least about 5 months, at least about 6 months, at least about 7 months, at least about 8 months, at least about 9 months, at least about 10 months, at least about 11 months, or at least about 1 year or more when compared to a reference individual (e.g., corresponding subject treated with IL-7 protein alone or with a bispecific antibody alone). In other aspects, the methods disclosed herein increases duration of survival of the subject at a level higher than (about one month higher than, about two months higher than, about three months higher than, about four months higher than, about five months higher than, about six months higher than, about seven months higher than, about eight months higher than, about nine months higher than, about ten months higher than, about eleven months higher than, or about one year higher than) the duration of survival of a reference subject (e.g., corresponding subject treated with IL-7 protein alone or with a bispecific antibody alone).

In some aspects, methods of the present disclosure effectively increase the duration of progression-free survival of a subject (e.g., cancer patient). For example, the progression free survival of the subject is increased by at least about 1 month, at least about 2 months, at least about 3 months, at least about 4 months, at least about 5 months, at least about 6 months, at least about 7 months, at least about 8 months, at least about 9 months, at least about 10 months, at least about 11 months, or at least about 1 year when compared to a reference subject (e.g., corresponding subject treated with IL-7 protein alone or with a bispecific antibody alone).

In some aspects, methods disclosed herein effectively increases the response rate in a group of subjects. For example, the response rate in a group of subjects is increased by at least about 2%, at least about 3%, at least about 4%, at least about 5%, at least about 10%, at least about 15%, at least about 20%, at least about 25%, at least about 30%, at last about 35%, at least about 40%, at least about 45%, at least about 50%, at least about 55%, at least about 60%, at least about 70%, at least about 75%, at least about 80%, at least about 85%, at least about 90%, at least about 95%, at least about 99% or at least about 100% when compared to a reference subject (e.g., corresponding subject treated with IL-7 protein alone or with a bispecific antibody alone).

In some aspects, the subject being treated in the method is a nonhuman animal, such as a rat or a mouse. In some aspects, the subject being treated in the method is a human.

In some aspects, the unit dose (e.g., for human use) of an IL-7 protein disclosed herein can be in the range of 0.001 mg/kg to 10 mg/kg. In certain aspects, the unit dose of an IL-7 protein is in the range of 0.01 mg/kg to 2 mg/kg. In some aspects, the unit dose is in the range of 0.02 mg/kg to 1 mg/kg. The unit dose can vary depending on the subject diseases for treatment and the presence of adverse effects. The administration of an IL-7 protein can be performed by periodic bolus injections or external reservoirs (e.g., intravenous bags) or by continuous intravenous, subcutaneous, or intraperitoneal administration from the internal (e.g., biocorrosive implants). In certain aspects, an IL-7 protein disclosed herein is administered via intramuscular injection.

In some aspects, an IL-7 protein disclosed herein can be administered to a subject at a weight-based dose. In certain aspects, an IL-7 protein can be administered at a weight-based dose between about 20 μg/kg and about 600 μg/kg. In further aspects, an IL-7 protein of the present disclosure can be administered at a weight-based dose of about 20 μg/kg, about 60 μg/kg, about 120 μg/kg, about 240 μg/kg, about 360 μg/kg, about 480 μg/kg, or about 600 μg/kg.

In some aspects, an IL-7 protein disclosed herein can be administered to a subject at a dose greater than about 600 μg/kg. In certain aspects, an IL-7 protein is administered to a subject at a dose greater than about 600 μg/kg, greater than about 700 μg/kg, greater than about 800 μg/kg, greater than about 900 μg/kg, greater than about 1,000 μg/kg, greater than about 1,100 μg/kg, greater than about 1,200 μg/kg, greater than about 1,300 μg/kg, greater than about 1,400 μg/kg, greater than about 1,500 μg/kg, greater than about 1,600 μg/kg, greater than about 1,700 μg/kg, greater than about 1,800 μg/kg, greater than about 1,900 μg/kg, or greater than about 2,000 μg/kg.

In some aspects, an IL-7 protein of the present disclosure is administered at a dose of between 610 μg/kg and about 1,200 μg/kg, between 650 μg/kg and about 1,200 μg/kg, between about 700 μg/kg and about 1,200 μg/kg, between about 750 μg/kg and about 1,200 μg/kg, between about 800 μg/kg and about 1,200 μg/kg, between about 850 μg/kg and about 1,200 μg/kg, between about 900 μg/kg and about 1,200 μg/kg, between about 950 μg/kg and about 1,200 μg/kg, between about 1,000 μg/kg and about 1,200 μg/kg, between about 1,050 μg/kg and about 1,200 μg/kg, between about 1,100 μg/kg and about 1,200 μg/kg, between about 1,200 μg/kg and about 2,000 μg/kg, between about 1,300 μg/kg and about 2,000 μg/kg, between about 1,500 μg/kg and about 2,000 μg/kg, between about 1,700 μg/kg and about 2,000 μg/kg, between about 610 μg/kg and about 1,000 μg/kg, between about 650 μg/kg and about 1,000 μg/kg, between about 700 μg/kg and about 1,000 μg/kg, between about 750 μg/kg and about 1,000 μg/kg, between about 800 μg/kg and about 1,000 μg/kg, between about 850 μg/kg and about 1,000 μg/kg, between about 900 μg/kg and about 1,000 μg/kg, or between about 950 μg/kg and about 1,000 μg/kg.

In some aspects, an IL-7 protein of the present disclosure is administered at a dose of between 610 μg/kg and about 1,200 μg/kg. In certain aspects, an IL-7 protein is administered at a dose of between 650 μg/kg and about 1,200 μg/kg. In some aspects, an IL-7 protein is administered at a dose of between about 700 μg/kg and about 1,200 μg/kg. In further aspects, an IL-7 protein is administered at a dose of between about 750 μg/kg and about 1,200 μg/kg. In certain aspects, an IL-7 protein is administered at a dose of between about 800 μg/kg and about 1,200 μg/kg. In some aspects, an IL-7 protein is administered at a dose of between about 850 μg/kg and about 1,200 μg/kg. In some aspects, an IL-7 protein is administered at a dose of between about 900 μg/kg and about 1,200 μg/kg. In further aspects, an IL-7 protein is administered at a dose of between about 950 μg/kg and about 1,200 μg/kg. In some aspects, an IL-7 protein disclosed herein is administered at a dose of between about 1,000 μg/kg and about 1,200 μg/kg. In some aspects, an IL-7 protein is administered at a dose of between about 1,050 μg/kg and about 1,200 μg/kg. In some aspects, an IL-7 protein is administered at a dose of between about 1,100 μg/kg and about 1,200 μg/kg. In some aspects, an IL-7 protein is administered at a dose of between about 1,200 μg/kg and about 2,000 μg/kg. In further aspects, an IL-7 protein is administered at a dose of between about 1,300 μg/kg and about 2,000 μg/kg. In some aspects, an IL-7 protein is administered at a dose of between about 1,500 μg/kg and about 2,000 μg/kg. In some aspects, an IL-7 protein is administered at a dose of between about 1,700 μg/kg and about 2,000 μg/kg. In certain aspects, an IL-7 protein is administered at a dose of between about 610 μg/kg and about 1,000 μg/kg. In some aspects, an IL-7 protein is administered at a dose of between about 650 μg/kg and about 1,000 μg/kg. In further aspects, an IL-7 protein is administered at a dose of between about 700 μg/kg and about 1,000 μg/kg. In yet further aspects, an IL-7 protein is administered at a dose of between about 750 μg/kg and about 1,000 μg/kg. In certain aspects, an IL-7 protein is administered at a dose of between about 800 μg/kg and about 1,000 μg/kg. In some aspects, an IL-7 protein is administered at a dose of between about 850 μg/kg and about 1,000 μg/kg. In some aspects, an IL-7 protein of the present disclosure is administered at a dose of between about 900 μg/kg and about 1,000 μg/kg. In some aspects, an IL-7 protein is administered at a dose of between about 950 μg/kg and about 1,000 μg/kg.

In some aspects, an IL-7 protein is administered at a dose of between about 700 μg/kg and about 900 μg/kg, between about 750 μg/kg and about 950 μg/kg, between about 700 μg/kg and about 850 μg/kg, between about 750 μg/kg and about 850 μg/kg, between about 700 μg/kg and about 800 μg/kg, between about 800 μg/kg and about 900 μg/kg, between about 750 μg/kg and about 850 μg/kg, or between about 850 μg/kg and about 950 μg/kg. In some aspects, an IL-7 protein is administered at a dose of between about 700 μg/kg and about 900 μg/kg. In certain aspects, an IL-7 protein is administered at a dose of between about 750 μg/kg and about 950 μg/kg. In further aspects, an IL-7 protein is administered at a dose of between about 700 μg/kg and about 850 μg/kg. In some aspects, an IL-7 protein is administered at a dose of between about 750 ⱼug/kg and about 850 μg/kg. In other aspects, an IL-7 protein is administered at a dose of between about 700 μg/kg and about 800 μg/kg. In some aspects, an IL-7 protein is administered at a dose of between about 800 μg/kg and about 900 μg/kg. In some aspects, an IL-7 protein is administered at a dose of between about 750 μg/kg and about 850 μg/kg. In certain aspects, an IL-7 protein is administered at a dose of between about 850 μg/kg and about 950 μg/kg.

In some aspects, an IL-7 protein is administered at a dose of about 650 μg/kg, about 680 μg/kg, about 700 μg/kg, about 720 μg/kg, about 740 μg/kg, about 750 μg/kg, about 760 μg/kg, about 780 μg/kg, about 800 μg/kg, about 820 μg/kg, about 840 μg/kg, about 850 μg/kg, about 860 μg/kg, about 880 μg/kg, about 900 μg/kg, about 920 μg/kg, about 940 μg/kg, about 950 μg/kg, about 960 μg/kg, about 980 μg/kg, about 1,000 μg/kg, about 1,020 μg/kg, about 1,020 μg/kg, about 1,040 μg/kg, about 1,060 μg/kg, about 1,080 μg/kg, about 1,100 μg/kg, about 1,120 μg/kg, about 1,140 μg/kg, about 1,160 μg/kg, about 1,180 μg/kg, about 1200 μg/kg, about 1,220 μg/kg, about 1,240 μg/kg, about 1,260 μg/kg, about 1,280 μg/kg, about 1,300 μg/kg, about 1,320 μg/kg, about 1,340 μg/kg, about 1,360 μg/kg, about 1,380 μg/kg, about 1,400 μg/kg, about 1,420 μg/kg, about 1,440 μg/kg, about 1,460 μg/kg, about 1,480 μg/kg, about 1,500 μg/kg, about 1,520 μg/kg, about 1,540 ⱼug/kg, about 1,560 μg/kg, about 1,580 μg/kg, about 1,600 μg/kg, about 1,620 μg/kg, about 1,640 μg/kg, about 1,660 μg/kg, about 1,680 μg/kg, about 1,700 μg/kg, about 1,720 μg/kg, about 1,740 μg/kg, about 1,760 μg/kg, about 1,780 μg/kg, about 1,800 μg/kg, about 1,820 μg/kg, about 1,840 μg/kg, about 1,860 μg/kg, about 1,880 μg/kg, about 1,900 μg/kg, about 1,920 μg/kg, about 1,940 μg/kg, about 1,960 μg/kg, about 1,980 μg/kg, or about 2,000 μg/kg.

In some aspects, an IL-7 protein is administered at a dose of about 650 μg/kg. In other aspects, an IL-7 protein disclosed herein is administered at a dose of about 680 μg/kg. In some aspects, an IL-7 protein is administered at a dose of about 700 μg/kg. In some aspects, an IL-7 protein is administered at a dose of about 720 μg/kg. In certain aspects, an IL-7 protein is administered at a dose of about 740 μg/kg. In some aspects, an IL-7 protein is administered at a dose of about 750 μg/kg. In some aspects, an IL-7 protein is administered at a dose of about 760 μg/kg. In certain aspects, an IL-7 protein is administered at a dose of about 780 μg/kg. In some aspects, an IL-7 protein is administered at a dose of about 800 μg/kg. In further aspects, an IL-7 protein is administered at a dose of about 820 μg/kg. In certain aspects, an IL-7 protein is administered at a dose of about 840 μg/kg. In some aspects, an IL-7 protein is administered at a dose of about 850 μg/kg. In certain aspects, an IL-7 protein is administered at a dose of about 860 μg/kg. In some aspects, an IL-7 protein is administered at a dose of about 880 μg/kg. In some aspects, an IL-7 protein is administered at a dose of about 900 μg/kg. In further aspects, an IL-7 protein is administered at a dose of about 920 μg/kg. In some aspects, an IL-7 protein is administered at a dose of about 940 μg/kg. In further aspects, an IL-7 protein is administered at a dose of about 950 μg/kg. In some aspects, an IL-7 protein is administered at a dose of about 960 μg/kg. In certain aspects, an IL-7 protein is administered at a dose of about 980 μg/kg. In some aspects, an IL-7 protein is administered at a dose of about 1,000 μg/kg. In certain aspects, an IL-7 protein is administered at a dose of about 1,020 μg/kg. In further aspects, an IL-7 protein is administered at a dose of about 1,040 μg/kg. In some aspects, an IL-7 protein is administered at a dose of about 1,060 μg/kg. In other aspects, an IL-7 protein is administered at a dose of about 1,080 μg/kg. In some aspects, an IL-7 protein is administered at a dose of about 1,100 μg/kg. In certain aspects, an IL-7 protein is administered at a dose of about 1,120 μg/kg. In further aspects, an IL-7 protein is administered at a dose of about 1,140 μg/kg. In some aspects, an IL-7 protein is administered at a dose of about 1,160 μg/kg. In other aspects, an IL-7 protein is administered at a dose of about 1,180 μg/kg. In certain aspects, an IL-7 protein is administered at a dose of about 1,200 μg/kg. In certain aspects, an IL-7 protein is administered at a dose of about 1,220 μg/kg. In further aspects, an IL-7 protein is administered at a dose of about 1,240 μg/kg. In some aspects, an IL-7 protein is administered at a dose of about 1,260 ⱼug/kg. In other aspects, an IL-7 protein is administered at a dose of about 1,280 μg/kg. In some aspects, an IL-7 protein is administered at a dose of about 1,300 μg/kg. In certain aspects, an IL-7 protein is administered at a dose of about 1,320 μg/kg. In further aspects, an IL-7 protein is administered at a dose of about 1,340 μg/kg. In some aspects, an IL-7 protein is administered at a dose of about 1,360 μg/kg. In other aspects, an IL-7 protein is administered at a dose of about 1,380 μg/kg. In further aspects, an IL-7 protein is administered at a dose of about 1,400 μg/kg. In certain aspects, an IL-7 protein is administered at a dose of about 1,420 μg/kg. In some aspects, an IL-7 protein is administered at a dose of about 1,440 μg/kg. In some aspects, an IL-7 protein is administered at a dose of about 1,460 μg/kg. In other aspects, an IL-7 protein is administered at a dose of about 1,480 μg/kg. In certain aspects, an IL-7 protein is administered at a dose of about 1,500 μg/kg. In certain aspects, an IL-7 protein is administered at a dose of about 1,520 μg/kg. In further aspects, an IL-7 protein is administered at a dose of about 1,540 μg/kg. In some aspects, an IL-7 protein is administered at a dose of about 1,560 μg/kg. In other aspects, an IL-7 protein is administered at a dose of about 1,580 μg/kg. In some aspects, an IL-7 protein is administered at a dose of about 1,600 μg/kg. In certain aspects, an IL-7 protein is administered at a dose of about 1,620 μg/kg. In further aspects, an IL-7 protein is administered at a dose of about 1,640 μg/kg. In some aspects, an IL-7 protein is administered at a dose of about 1,660 μg/kg. In other aspects, an IL-7 protein is administered at a dose of about 1,680 μg/kg. In certain aspects, an IL-7 protein is administered at a dose of about 1,700 μg/kg. In certain aspects, an IL-7 protein is administered at a dose of about 1,720 μg/kg. In further aspects, an IL-7 protein is administered at a dose of about 1,740 μg/kg. In some aspects, an IL-7 protein is administered at a dose of about 1,760 μg/kg. In other aspects, an IL-7 protein is administered at a dose of about 1,780 μg/kg. In some aspects, an IL-7 protein is administered at a dose of about 1,800 μg/kg. In certain aspects, an IL-7 protein is administered at a dose of about 1,820 μg/kg. In further aspects, an IL-7 protein is administered at a dose of about 1,840 μg/kg. In some aspects, an IL-7 protein is administered at a dose of about 1,860 μg/kg. In other aspects, an IL-7 protein is administered at a dose of about 1,880 μg/kg. In some aspects, an IL-7 protein is administered at a dose of about 1,900 μg/kg. In certain aspects, an IL-7 protein is administered at a dose of about 1,920 μg/kg. In further aspects, an IL-7 protein is administered at a dose of about 1,940 μg/kg. In some aspects, an IL-7 protein is administered at a dose of about 1,960 μg/kg. In other aspects, an IL-7 protein is administered at a dose of about 1,980 μg/kg. In further aspects, an IL-7 protein is administered at a dose of about 2,000 μg/kg.

In some aspects, an IL-7 protein can be administered at a flat dose. In certain aspects, an IL-7 protein can be administered at a flat dose of about 0.25 mg to about 9 mg. In some aspects, an IL-7 protein can be administered at a flat dose of about 0.25 mg, about 1 mg, about 3 mg, about 6 mg, or about 9 mg.

In some aspects, an IL-7 protein disclosed herein is administered to a subject at multiple doses (i.e., repeated administrations). In certain embodiments, an IL-7 protein is administered to the subject at least two times, at least three times, at least four times, at least five times, at least six times, at least seven times, at least eight times, at least nine times, or at least ten times or more. In other embodiments, a subject receives a single administration of the IL-7 protein (e.g., prior to, concurrently, or after an administration of an immune checkpoint inhibitor).

In some aspects, an IL-7 protein is administered at a dosing frequency of about once a week, about once in two weeks, about once in three weeks, about once in four weeks, about once in five weeks, about once in six weeks, about once in seven weeks, about once in eight weeks, about once in nine weeks, about once in 10 weeks, about once in 11 weeks, or about once in 12 weeks. In certain aspects, an IL-7 protein is administered at a dosing frequency of about once every 10 days, about once every 20 days, about once every 30 days, about once every 40 days, about once every 50 days, about once every 60 days, about once every 70 days, about once every 80 days, about once every 90 days, or about once every 100 days. In some aspects, the IL-7 protein is administered once in three weeks. In some aspects, the IL-7 protein is administered once a week. In some aspects, the IL-7 protein is administered once in two weeks. In certain aspects, the IL-7 protein is administered once in three weeks. In some aspects, the IL-7 protein is administered once in four weeks. In certain aspects, the IL-7 protein is administered once in six weeks. In further aspects, the IL-7 protein is administered once in eight weeks. In some aspects, the IL-7 protein is administered once in nine weeks. In certain aspects, the IL-7 protein is administered once in 12 weeks. In some aspects, the IL-7 protein is administered once every 10 days. In certain aspects, the IL-7 protein is administered once every 20 days. In other aspects, the IL-7 protein is administered once every 30 days. In some aspects, the IL-7 protein is administered once every 40 days. In certain aspects, the IL-7 protein is administered once every 50 days. In some aspects, the IL-7 protein is administered once every 60 days. In further aspects, the IL-7 protein is administered once every 70 days. In some aspects, the IL-7 protein is administered once every 80 days. In certain aspects, the IL-7 protein is administered once every 90 days. In some aspects, the IL-7 protein is administered once every 100 days.

In some aspects, the IL-7 protein is administered twice or more times in an amount of about 720 μg/kg at an interval of about 1 week, about 2 weeks, about 3 weeks, or about 4 weeks. In some aspects, the IL-7 protein is administered twice or more times in an amount of about 840 μg/kg at an interval of about 2 weeks, about 3 weeks, about 4 weeks, or about 5 weeks. In some aspects, the IL-7 protein is administered twice or more times in an amount of about 960 μg/kg at an interval of about 2 weeks, about 3 weeks, about 4 weeks, about 5 weeks, or about 6 weeks. In some aspects, the IL-7 protein is administered twice or more times in an amount of about 1200 μg/kg at an interval of about 3 weeks, about 4 weeks, about 5 weeks, about 6 weeks, about 7 weeks, or about 8 weeks. In some aspects, the IL-7 protein is administered twice or more times in an amount of about 1440 μg/kg at an interval of about 3 weeks, about 4 weeks, about 5 weeks, about 6 weeks, about 7 weeks, about 8 weeks, about 2 months, about 8 weeks, about 10 weeks, about 12 weeks, or about 3 months.

In some aspects, the IL-7 protein is administered at a dose of 60 μg/kg with a dosing frequency of once a week. In some aspects, the IL-7 protein is administered at a dose of 120 μg/kg with a dosing frequency of once a week. In some aspects, the IL-7 protein is administered at a dose of 240 μg/kg with a dosing frequency of once a week. In some aspects, the IL-7 protein is administered at a dose of 480 μg/kg with a dosing frequency of once a week. In some aspects, the IL-7 protein is administered at a dose of 720 μg/kg with a dosing frequency of once a week. In some aspects, the IL-7 protein is administered at a dose of 960 μg/kg with a dosing frequency of once a week. In some aspects, the IL-7 protein is administered at a dose of 1,200 μg/kg with a dosing frequency of once a week. In some aspects, the IL-7 protein is administered at a dose of 1,300 μg/kg with a dosing frequency of once a week. In some aspects, the IL-7 protein is administered at a dose of 1,400 μg/kg with a dosing frequency of once a week. In other aspects, the IL-7 protein is administered at a dose of 1,420 μg/kg with a dosing frequency of once a week. In certain aspects, the IL-7 protein is administered at a dose of 1,440 μg/kg with a dosing frequency of once a week. In further aspects, the IL-7 protein is administered at a dose of 1,460 μg/kg with a dosing frequency of once a week. In certain aspects, the IL-7 protein is administered at a dose of 1,480 μg/kg with a dosing frequency of once a week. In some aspects, the IL-7 protein is administered at a dose of 1,500 μg/kg with a dosing frequency of once a week. In further aspects, the IL-7 protein is administered at a dose of 1,600 μg/kg with a dosing frequency of once a week. In some aspects, the IL-7 protein is administered at a dose of 1,700 μg/kg with a dosing frequency of once a week. In some aspects, the IL-7 protein is administered at a dose of 2,000 μg/kg with a dosing frequency of once a week.

In some aspects, the IL-7 protein is administered at a dose of 60 μg/kg with a dosing frequency of once in two weeks. In some aspects, the IL-7 protein is administered at a dose of 120 μg/kg with a dosing frequency of once in two weeks. In some aspects, the IL-7 protein is administered at a dose of 240 μg/kg with a dosing frequency of once in two weeks. In some aspects, the IL-7 protein is administered at a dose of 480 μg/kg with a dosing frequency of once in two weeks. In some aspects, the IL-7 protein is administered at a dose of 720 μg/kg with a dosing frequency of once in two weeks. In some aspects, the IL-7 protein is administered at a dose of 960 μg/kg with a dosing frequency of once in two weeks. In some aspects, the IL-7 protein is administered at a dose of 1,200 μg/kg with a dosing frequency of once in two weeks. In some aspects, the IL-7 protein is administered at a dose of 1,300 μg/kg with a dosing frequency of once in two weeks. In some aspects, the IL-7 protein is administered at a dose of 1,400 μg/kg with a dosing frequency of once in two weeks. In other aspects, the IL-7 protein is administered at a dose of 1,420 μg/kg with a dosing frequency of once in two weeks. In certain aspects, the IL-7 protein is administered at a dose of 1,440 μg/kg with a dosing frequency of once in two weeks. In further aspects, the IL-7 protein is administered at a dose of 1,460 μg/kg with a dosing frequency of once in two weeks. In certain aspects, the IL-7 protein is administered at a dose of 1,480 μg/kg with a dosing frequency of once in two weeks. In some aspects, the IL-7 protein is administered at a dose of 1,500 μg/kg with a dosing frequency of once in two weeks. In further aspects, the IL-7 protein is administered at a dose of 1,600 μg/kg with a dosing frequency of once in two weeks. In some aspects, the IL-7 protein is administered at a dose of 1,700 μg/kg with a dosing frequency of once in two weeks. In some aspects, the IL-7 protein is administered at a dose of 2,000 μg/kg with a dosing frequency of once in two weeks.

In some aspects, the IL-7 protein is administered at a dose of 60 μg/kg with a dosing frequency of once in three weeks. In some aspects, the IL-7 protein is administered at a dose of 120 μg/kg with a dosing frequency of once in three weeks. In some aspects, the IL-7 protein is administered at a dose of 240 μg/kg with a dosing frequency of once in three weeks. In some aspects, the IL-7 protein is administered at a dose of 480 μg/kg with a dosing frequency of once in three weeks. In some aspects, the IL-7 protein is administered at a dose of 720 μg/kg with a dosing frequency of once in three weeks. In some aspects, the IL-7 protein is administered at a dose of 960 μg/kg with a dosing frequency of once in three weeks. In some aspects, the IL-7 protein is administered at a dose of 1,200 μg/kg with a dosing frequency of once in three weeks. In some aspects, the IL-7 protein is administered at a dose of 1,300 μg/kg with a dosing frequency of once in three weeks. In some aspects, the IL-7 protein is administered at a dose of 1,400 μg/kg with a dosing frequency of once in three weeks. In other aspects, the IL-7 protein is administered at a dose of 1,420 μg/kg with a dosing frequency of once in three weeks. In certain aspects, the IL-7 protein is administered at a dose of 1,440 μg/kg with a dosing frequency of once in three weeks. In further aspects, the IL-7 protein is administered at a dose of 1,460 μg/kg with a dosing frequency of once in three weeks. In certain aspects, the IL-7 protein is administered at a dose of 1,480 μg/kg with a dosing frequency of once in three weeks. In some aspects, the IL-7 protein is administered at a dose of 1,500 μg/kg with a dosing frequency of once in three weeks. In further aspects, the IL-7 protein is administered at a dose of 1,600 μg/kg with a dosing frequency of once in three weeks. In some aspects, the IL-7 protein is administered at a dose of 1,700 μg/kg with a dosing frequency of once in three weeks. In some aspects, the IL-7 protein is administered at a dose of 2,000 μg/kg with a dosing frequency of once in three weeks.

In some aspects, the IL-7 protein is administered at a dose of 60 μg/kg with a dosing frequency of once in four weeks. In some aspects, the IL-7 protein is administered at a dose of 120 μg/kg with a dosing frequency of once in four weeks. In some aspects, the IL-7 protein is administered at a dose of 240 μg/kg with a dosing frequency of once in four weeks. In some aspects, the IL-7 protein is administered at a dose of 480 μg/kg with a dosing frequency of once in four weeks. In some aspects, the IL-7 protein is administered at a dose of 720 μg/kg with a dosing frequency of once in four weeks. In some aspects, the IL-7 protein is administered at a dose of 960 μg/kg with a dosing frequency of once in four weeks. In some aspects, the IL-7 protein is administered at a dose of 1,200 μg/kg with a dosing frequency of once in four weeks. In some aspects, the IL-7 protein is administered at a dose of 1,300 μg/kg with a dosing frequency of once in four weeks. In some aspects, the IL-7 protein is administered at a dose of 1,400 μg/kg with a dosing frequency of once in four weeks. In other aspects, the IL-7 protein is administered at a dose of 1,420 μg/kg with a dosing frequency of once in four weeks. In certain aspects, the IL-7 protein is administered at a dose of 1,440 μg/kg with a dosing frequency of once in four weeks. In further aspects, the IL-7 protein is administered at a dose of 1,460 μg/kg with a dosing frequency of once in four weeks. In certain aspects, the IL-7 protein is administered at a dose of 1,480 μg/kg with a dosing frequency of once in four weeks. In some aspects, the IL-7 protein is administered at a dose of 1,500 μg/kg with a dosing frequency of once in four weeks. In further aspects, the IL-7 protein is administered at a dose of 1,600 μg/kg with a dosing frequency of once in four weeks. In some aspects, the IL-7 protein is administered at a dose of 1,700 μg/kg with a dosing frequency of once in four weeks. In some aspects, the IL-7 protein is administered at a dose of 2,000 μg/kg with a dosing frequency of once in four weeks.

In some aspects, the IL-7 protein is administered at a dose of 60 μg/kg with a dosing frequency of once in five weeks. In some aspects, the IL-7 protein is administered at a dose of 120 μg/kg with a dosing frequency of once in five weeks. In some aspects, the IL-7 protein is administered at a dose of 240 μg/kg with a dosing frequency of once in five weeks. In some aspects, the IL-7 protein is administered at a dose of 480 μg/kg with a dosing frequency of once in five weeks. In some aspects, the IL-7 protein is administered at a dose of 720 μg/kg with a dosing frequency of once in five weeks. In some aspects, the IL-7 protein is administered at a dose of 960 μg/kg with a dosing frequency of once in five weeks. In some aspects, the IL-7 protein is administered at a dose of 1,200 μg/kg with a dosing frequency of once in five weeks. In some aspects, the IL-7 protein is administered at a dose of 1,300 μg/kg with a dosing frequency of once in five weeks. In some aspects, the IL-7 protein is administered at a dose of 1,400 μg/kg with a dosing frequency of once in five weeks. In other aspects, the IL-7 protein is administered at a dose of 1,420 μg/kg with a dosing frequency of once in five weeks. In certain aspects, the IL-7 protein is administered at a dose of 1,440 μg/kg with a dosing frequency of once in five weeks. In further aspects, the IL-7 protein is administered at a dose of 1,460 μg/kg with a dosing frequency of once in five weeks. In certain aspects, the IL-7 protein is administered at a dose of 1,480 μg/kg with a dosing frequency of once in five weeks. In some aspects, the IL-7 protein is administered at a dose of 1,500 μg/kg with a dosing frequency of once in five weeks. In further aspects, the IL-7 protein is administered at a dose of 1,600 μg/kg with a dosing frequency of once in five weeks. In some aspects, the IL-7 protein is administered at a dose of 1,700 μg/kg with a dosing frequency of once in five weeks. In some aspects, the IL-7 protein is administered at a dose of 2,000 μg/kg with a dosing frequency of once in five weeks.

In some aspects, the IL-7 protein is administered at a dose of 60 μg/kg with a dosing frequency of once in six weeks. In some aspects, the IL-7 protein is administered at a dose of 120 μg/kg with a dosing frequency of once in six weeks. In some aspects, the IL-7 protein is administered at a dose of 240 μg/kg with a dosing frequency of once in six weeks. In some aspects, the IL-7 protein is administered at a dose of 480 μg/kg with a dosing frequency of once in six weeks. In some aspects, the IL-7 protein is administered at a dose of 720 μg/kg with a dosing frequency of once in six weeks. In some aspects, the IL-7 protein is administered at a dose of 960 μg/kg with a dosing frequency of once in six weeks. In some aspects, the IL-7 protein is administered at a dose of 1,200 μg/kg with a dosing frequency of once in six weeks. In some aspects, the IL-7 protein is administered at a dose of 1,300 μg/kg with a dosing frequency of once in six weeks. In some aspects, the IL-7 protein is administered at a dose of 1,400 μg/kg with a dosing frequency of once in six weeks. In other aspects, the IL-7 protein is administered at a dose of 1,420 μg/kg with a dosing frequency of once in six weeks. In certain aspects, the IL-7 protein is administered at a dose of 1,440 μg/kg with a dosing frequency of once in six weeks. In further aspects, the IL-7 protein is administered at a dose of 1,460 μg/kg with a dosing frequency of once in six weeks. In certain aspects, the IL-7 protein is administered at a dose of 1,480 μg/kg with a dosing frequency of once in six weeks. In some aspects, the IL-7 protein is administered at a dose of 1,500 μg/kg with a dosing frequency of once in six weeks. In further aspects, the IL-7 protein is administered at a dose of 1,600 μg/kg with a dosing frequency of once in six weeks. In some aspects, the IL-7 protein is administered at a dose of 1,700 μg/kg with a dosing frequency of once in six weeks. In some aspects, the IL-7 protein is administered at a dose of 2,000 μg/kg with a dosing frequency of once in six weeks.

In some aspects, the IL-7 protein is administered at a dose of 60 μg/kg with a dosing frequency of once in seven weeks. In some aspects, the IL-7 protein is administered at a dose of 120 μg/kg with a dosing frequency of once in seven weeks. In some aspects, the IL-7 protein is administered at a dose of 240 μg/kg with a dosing frequency of once in seven weeks. In some aspects, the IL-7 protein is administered at a dose of 480 μg/kg with a dosing frequency of once in seven weeks. In some aspects, the IL-7 protein is administered at a dose of 720 μg/kg with a dosing frequency of once in seven weeks. In some aspects, the IL-7 protein is administered at a dose of 960 μg/kg with a dosing frequency of once in seven weeks. In some aspects, the IL-7 protein is administered at a dose of 1,200 μg/kg with a dosing frequency of once in seven weeks. In some aspects, the IL-7 protein is administered at a dose of 1,300 μg/kg with a dosing frequency of once in seven weeks. In some aspects, the IL-7 protein is administered at a dose of 1,400 μg/kg with a dosing frequency of once in seven weeks. In other aspects, the IL-7 protein is administered at a dose of 1,420 μg/kg with a dosing frequency of once in seven weeks. In certain aspects, the IL-7 protein is administered at a dose of 1,440 μg/kg with a dosing frequency of once in seven weeks. In further aspects, the IL-7 protein is administered at a dose of 1,460 μg/kg with a dosing frequency of once in seven weeks. In certain aspects, the IL-7 protein is administered at a dose of 1,480 μg/kg with a dosing frequency of once in seven weeks. In some aspects, the IL-7 protein is administered at a dose of 1,500 μg/kg with a dosing frequency of once in seven weeks. In further aspects, the IL-7 protein is administered at a dose of 1,600 μg/kg with a dosing frequency of once in seven weeks. In some aspects, the IL-7 protein is administered at a dose of 1,700 μg/kg with a dosing frequency of once in seven weeks. In some aspects, the IL-7 protein is administered at a dose of 2,000 μg/kg with a dosing frequency of once in seven weeks.

In some aspects, the IL-7 protein is administered at a dose of 60 μg/kg with a dosing frequency of once in eight weeks. In some aspects, the IL-7 protein is administered at a dose of 120 μg/kg with a dosing frequency of once in eight weeks. In some aspects, the IL-7 protein is administered at a dose of 240 μg/kg with a dosing frequency of once in eight weeks. In some aspects, the IL-7 protein is administered at a dose of 480 μg/kg with a dosing frequency of once in eight weeks. In some aspects, the IL-7 protein is administered at a dose of 720 μg/kg with a dosing frequency of once in eight weeks. In some aspects, the IL-7 protein is administered at a dose of 960 μg/kg with a dosing frequency of once in eight weeks. In some aspects, the IL-7 protein is administered at a dose of 1,200 μg/kg with a dosing frequency of once in eight weeks. In some aspects, the IL-7 protein is administered at a dose of 1,300 μg/kg with a dosing frequency of once in eight weeks. In some aspects, the IL-7 protein is administered at a dose of 1,400 μg/kg with a dosing frequency of once in eight weeks. In other aspects, the IL-7 protein is administered at a dose of 1,420 μg/kg with a dosing frequency of once in eight weeks. In certain aspects, the IL-7 protein is administered at a dose of 1,440 μg/kg with a dosing frequency of once in eight weeks. In further aspects, the IL-7 protein is administered at a dose of 1,460 μg/kg with a dosing frequency of once in eight weeks. In certain aspects, the IL-7 protein is administered at a dose of 1,480 μg/kg with a dosing frequency of once in eight weeks. In some aspects, the IL-7 protein is administered at a dose of 1,500 μg/kg with a dosing frequency of once in eight weeks. In further aspects, the IL-7 protein is administered at a dose of 1,600 μg/kg with a dosing frequency of once in eight weeks. In some aspects, the IL-7 protein is administered at a dose of 1,700 μg/kg with a dosing frequency of once in eight weeks. In some aspects, the IL-7 protein is administered at a dose of 2,000 μg/kg with a dosing frequency of once in eight weeks.

In some aspects, the IL-7 protein is administered at a dose of 60 μg/kg with a dosing frequency of once in nine weeks. In some aspects, the IL-7 protein is administered at a dose of 120 μg/kg with a dosing frequency of once in nine weeks. In some aspects, the IL-7 protein is administered at a dose of 240 μg/kg with a dosing frequency of once in nine weeks. In some aspects, the IL-7 protein is administered at a dose of 480 μg/kg with a dosing frequency of once in nine weeks. In some aspects, the IL-7 protein is administered at a dose of 720 μg/kg with a dosing frequency of once in nine weeks. In some aspects, the IL-7 protein is administered at a dose of 960 μg/kg with a dosing frequency of once in nine weeks. In some aspects, the IL-7 protein is administered at a dose of 1,200 μg/kg with a dosing frequency of once in nine weeks. In some aspects, the IL-7 protein is administered at a dose of 1,300 μg/kg with a dosing frequency of once in three weeks. In some aspects, the IL-7 protein is administered at a dose of 1,400 μg/kg with a dosing frequency of once in three weeks. In other aspects, the IL-7 protein is administered at a dose of 1,420 μg/kg with a dosing frequency of once in three weeks. In certain aspects, the IL-7 protein is administered at a dose of 1,440 μg/kg with a dosing frequency of once in nine weeks. In further aspects, the IL-7 protein is administered at a dose of 1,460 μg/kg with a dosing frequency of once in three weeks. In certain aspects, the IL-7 protein is administered at a dose of 1,480 μg/kg with a dosing frequency of once in three weeks. In some aspects, the IL-7 protein is administered at a dose of 1,500 μg/kg with a dosing frequency of once in three weeks. In further aspects, the IL-7 protein is administered at a dose of 1,600 μg/kg with a dosing frequency of once in three weeks. In some aspects, the IL-7 protein is administered at a dose of 1,700 μg/kg with a dosing frequency of once in nine weeks. In some aspects, the IL-7 protein is administered at a dose of 2,000 μg/kg with a dosing frequency of once in nine weeks.

In some aspects, the IL-7 protein is administered at a dose of 60 μg/kg with a dosing frequency of once in 10 weeks. In some aspects, the IL-7 protein is administered at a dose of 120 μg/kg with a dosing frequency of once in 10 weeks. In some aspects, the IL-7 protein is administered at a dose of 240 μg/kg with a dosing frequency of once in 10 weeks. In some aspects, the IL-7 protein is administered at a dose of 480 μg/kg with a dosing frequency of once in 10 weeks. In some aspects, the IL-7 protein is administered at a dose of 720 μg/kg with a dosing frequency of once in 10 weeks. In some aspects, the IL-7 protein is administered at a dose of 960 μg/kg with a dosing frequency of once in 10 weeks. In some aspects, the IL-7 protein is administered at a dose of 1,200 μg/kg with a dosing frequency of once in 10 weeks. In some aspects, the IL-7 protein is administered at a dose of 1,300 μg/kg with a dosing frequency of once in 10 weeks. In some aspects, the IL-7 protein is administered at a dose of 1,400 μg/kg with a dosing frequency of once in 10 weeks. In other aspects, the IL-7 protein is administered at a dose of 1,420 μg/kg with a dosing frequency of once in 10 weeks. In certain aspects, the IL-7 protein is administered at a dose of 1,440 μg/kg with a dosing frequency of once in 10 weeks. In further aspects, the IL-7 protein is administered at a dose of 1,460 μg/kg with a dosing frequency of once in 10 weeks. In certain aspects, the IL-7 protein is administered at a dose of 1,480 μg/kg with a dosing frequency of once in 10 weeks. In some aspects, the IL-7 protein is administered at a dose of 1,500 μg/kg with a dosing frequency of once in 10 weeks. In further aspects, the IL-7 protein is administered at a dose of 1,600 μg/kg with a dosing frequency of once in 10 weeks. In some aspects, the IL-7 protein is administered at a dose of 1,700 μg/kg with a dosing frequency of once in 10 weeks. In some aspects, the IL-7 protein is administered at a dose of 2,000 μg/kg with a dosing frequency of once in 10 weeks.

In some aspects, the IL-7 protein is administered at a dose of 60 μg/kg with a dosing frequency of once in 11 weeks. In some aspects, the IL-7 protein is administered at a dose of 120 μg/kg with a dosing frequency of once in 11 weeks. In some aspects, the IL-7 protein is administered at a dose of 240 μg/kg with a dosing frequency of once in 11 weeks. In some aspects, the IL-7 protein is administered at a dose of 480 μg/kg with a dosing frequency of once in 11 weeks. In some aspects, the IL-7 protein is administered at a dose of 720 _{K}g/kg with a dosing frequency of once in 11 weeks. In some aspects, the IL-7 protein is administered at a dose of 960 μg/kg with a dosing frequency of once in 11 weeks. In some aspects, the IL-7 protein is administered at a dose of 1,200 μg/kg with a dosing frequency of once in 11 weeks. In some aspects, the IL-7 protein is administered at a dose of 1,300 μg/kg with a dosing frequency of once in 11 weeks. In some aspects, the IL-7 protein is administered at a dose of 1,400 μg/kg with a dosing frequency of once in 11 weeks. In other aspects, the IL-7 protein is administered at a dose of 1,420 ⱼug/kg with a dosing frequency of once in 11 weeks. In certain aspects, the IL-7 protein is administered at a dose of 1,440 μg/kg with a dosing frequency of once in 11 weeks. In further aspects, the IL-7 protein is administered at a dose of 1,460 μg/kg with a dosing frequency of once in 11 weeks. In certain aspects, the IL-7 protein is administered at a dose of 1,480 μg/kg with a dosing frequency of once in 11 weeks. In some aspects, the IL-7 protein is administered at a dose of 1,500 μg/kg with a dosing frequency of once in 11 weeks. In further aspects, the IL-7 protein is administered at a dose of 1,600 μg/kg with a dosing frequency of once in 11 weeks. In some aspects, the IL-7 protein is administered at a dose of 1,700 μg/kg with a dosing frequency of once in 11 weeks. In some aspects, the IL-7 protein is administered at a dose of 2,000 μg/kg with a dosing frequency of once in 11 weeks.

In some aspects, the IL-7 protein is administered at a dose of 60 μg/kg with a dosing frequency of once in 12 weeks. In some aspects, the IL-7 protein is administered at a dose of 120 μg/kg with a dosing frequency of once in 12 weeks. In some aspects, the IL-7 protein is administered at a dose of 240 μg/kg with a dosing frequency of once in 12 weeks. In some aspects, the IL-7 protein is administered at a dose of 480 μg/kg with a dosing frequency of once in 12 weeks. In some aspects, the IL-7 protein is administered at a dose of 720 μg/kg with a dosing frequency of once in 12 weeks. In some aspects, the IL-7 protein is administered at a dose of 960 μg/kg with a dosing frequency of once in 12 weeks. In some aspects, the IL-7 protein is administered at a dose of 1,200 μg/kg with a dosing frequency of once in 12 weeks. In some aspects, the IL-7 protein is administered at a dose of 1,300 μg/kg with a dosing frequency of once in 12 weeks. In some aspects, the IL-7 protein is administered at a dose of 1,400 μg/kg with a dosing frequency of once in 12 weeks. In other aspects, the IL-7 protein is administered at a dose of 1,420 μg/kg with a dosing frequency of once in 12 weeks. In certain aspects, the IL-7 protein is administered at a dose of 1,440 μg/kg with a dosing frequency of once in 12 weeks. In further aspects, the IL-7 protein is administered at a dose of 1,460 μg/kg with a dosing frequency of once in 12 weeks. In certain aspects, the IL-7 protein is administered at a dose of 1,480 μg/kg with a dosing frequency of once in 12 weeks. In some aspects, the IL-7 protein is administered at a dose of 1,500 μg/kg with a dosing frequency of once in 12 weeks. In further aspects, the IL-7 protein is administered at a dose of 1,600 μg/kg with a dosing frequency of once in 12 weeks. In some aspects, the IL-7 protein is administered at a dose of 1,700 μg/kg with a dosing frequency of once in 12 weeks. In some aspects, the IL-7 protein is administered at a dose of 2,000 μg/kg with a dosing frequency of once in 12 weeks.

In some aspects, the IL-7 protein is administered at a dose of 60 μg/kg with a dosing frequency of once every 10 days. In some aspects, the IL-7 protein is administered at a dose of 120 μg/kg with a dosing frequency of once every 10 days. In some aspects, the IL-7 protein is administered at a dose of 240 μg/kg with a dosing frequency of once every 10 days. In some aspects, the IL-7 protein is administered at a dose of 480 μg/kg with a dosing frequency of once every 10 days. In some aspects, the IL-7 protein is administered at a dose of 720 μg/kg with a dosing frequency of once every 10 days. In some aspects, the IL-7 protein is administered at a dose of 960 μg/kg with a dosing frequency of once every 10 days. In some aspects, the IL-7 protein is administered at a dose of 1,200 μg/kg with a dosing frequency of once every 10 days. In some aspects, the IL-7 protein is administered at a dose of 1,300 μg/kg with a dosing frequency of once every 10 days. In some aspects, the IL-7 protein is administered at a dose of 1,400 μg/kg with a dosing frequency of once every 10 days. In other aspects, the IL-7 protein is administered at a dose of 1,420 μg/kg with a dosing frequency of once every 10 days. In certain aspects, the IL-7 protein is administered at a dose of 1,440 μg/kg with a dosing frequency of once every 10 days. In further aspects, the IL-7 protein is administered at a dose of 1,460 μg/kg with a dosing frequency of once every 10 days. In certain aspects, the IL-7 protein is administered at a dose of 1,480 μg/kg with a dosing frequency of once every 10 days. In some aspects, the IL-7 protein is administered at a dose of 1,500 μg/kg with a dosing frequency of once every 10 days. In further aspects, the IL-7 protein is administered at a dose of 1,600 μg/kg with a dosing frequency of once every 10 days. In some aspects, the IL-7 protein is administered at a dose of 1,700 μg/kg with a dosing frequency of once every 10 days. In some aspects, the IL-7 protein is administered at a dose of 2,000 μg/kg with a dosing frequency of once every 10 days.

In some aspects, the IL-7 protein is administered at a dose of 60 μg/kg with a dosing frequency of once every 20 days. In some aspects, the IL-7 protein is administered at a dose of 120 μg/kg with a dosing frequency of once every 20 days. In some aspects, the IL-7 protein is administered at a dose of 240 μg/kg with a dosing frequency of once every 20 days. In some aspects, the IL-7 protein is administered at a dose of 480 μg/kg with a dosing frequency of once every 20 days. In some aspects, the IL-7 protein is administered at a dose of 720 μg/kg with a dosing frequency of once every 20 days. In some aspects, the IL-7 protein is administered at a dose of 960 μg/kg with a dosing frequency of once every 20 days. In some aspects, the IL-7 protein is administered at a dose of 1,200 μg/kg with a dosing frequency of once every 20 days. In some aspects, the IL-7 protein is administered at a dose of 1,300 μg/kg with a dosing frequency of once every 20 days. In some aspects, the IL-7 protein is administered at a dose of 1,400 μg/kg with a dosing frequency of once every 20 days. In other aspects, the IL-7 protein is administered at a dose of 1,420 μg/kg with a dosing frequency of once every 20 days. In certain aspects, the IL-7 protein is administered at a dose of 1,440 μg/kg with a dosing frequency of once every 20 days. In further aspects, the IL-7 protein is administered at a dose of 1,460 μg/kg with a dosing frequency of once every 20 days. In certain aspects, the IL-7 protein is administered at a dose of 1,480 μg/kg with a dosing frequency of once every 20 days. In some aspects, the IL-7 protein is administered at a dose of 1,500 μg/kg with a dosing frequency of once every 20 days. In further aspects, the IL-7 protein is administered at a dose of 1,600 μg/kg with a dosing frequency of once every 20 days. In some aspects, the IL-7 protein is administered at a dose of 1,700 μg/kg with a dosing frequency of once every 20 days. In some aspects, the IL-7 protein is administered at a dose of 2,000 μg/kg with a dosing frequency of once every 20 days.

In some aspects, the IL-7 protein is administered at a dose of 60 μg/kg with a dosing frequency of once every 30 days. In some aspects, the IL-7 protein is administered at a dose of 120 μg/kg with a dosing frequency of once every 30 days. In some aspects, the IL-7 protein is administered at a dose of 240 μg/kg with a dosing frequency of once every 30 days. In some aspects, the IL-7 protein is administered at a dose of 480 μg/kg with a dosing frequency of once every 30 days. In some aspects, the IL-7 protein is administered at a dose of 720 μg/kg with a dosing frequency of once every 30 days. In some aspects, the IL-7 protein is administered at a dose of 960 μg/kg with a dosing frequency of once every 30 days. In some aspects, the IL-7 protein is administered at a dose of 1,200 μg/kg with a dosing frequency of once every 30 days. In some aspects, the IL-7 protein is administered at a dose of 1,300 μg/kg with a dosing frequency of once every 30 days. In some aspects, the IL-7 protein is administered at a dose of 1,400 μg/kg with a dosing frequency of once every 30 days. In other aspects, the IL-7 protein is administered at a dose of 1,420 μg/kg with a dosing frequency of once every 30 days. In certain aspects, the IL-7 protein is administered at a dose of 1,440 μg/kg with a dosing frequency of once every 30 days. In further aspects, the IL-7 protein is administered at a dose of 1,460 μg/kg with a dosing frequency of once every 30 days. In certain aspects, the IL-7 protein is administered at a dose of 1,480 μg/kg with a dosing frequency of once every 30 days. In some aspects, the IL-7 protein is administered at a dose of 1,500 μg/kg with a dosing frequency of once every 30 days. In further aspects, the IL-7 protein is administered at a dose of 1,600 μg/kg with a dosing frequency of once every 30 days. In some aspects, the IL-7 protein is administered at a dose of 1,700 μg/kg with a dosing frequency of once every 30 days. In some aspects, the IL-7 protein is administered at a dose of 2,000 μg/kg with a dosing frequency of once every 30 days.

In some aspects, the IL-7 protein is administered at a dose of 60 _{K}g/kg with a dosing frequency of once every 40 days. In some aspects, the IL-7 protein is administered at a dose of 120 μg/kg with a dosing frequency of once every 40 days. In some aspects, the IL-7 protein is administered at a dose of 240 μg/kg with a dosing frequency of once every 40 days. In some aspects, the IL-7 protein is administered at a dose of 480 μg/kg with a dosing frequency of once every 40 days. In some aspects, the IL-7 protein is administered at a dose of 720 μg/kg with a dosing frequency of once every 40 days. In some aspects, the IL-7 protein is administered at a dose of 960 μg/kg with a dosing frequency of once every 40 days. In some aspects, the IL-7 protein is administered at a dose of 1,200 μg/kg with a dosing frequency of once every 40 days. In some aspects, the IL-7 protein is administered at a dose of 1,300 μg/kg with a dosing frequency of once every 40 days. In some aspects, the IL-7 protein is administered at a dose of 1,400 μg/kg with a dosing frequency of once every 40 days. In other aspects, the IL-7 protein is administered at a dose of 1,420 μg/kg with a dosing frequency of once every 40 days. In certain aspects, the IL-7 protein is administered at a dose of 1,440 μg/kg with a dosing frequency of once every 40 days. In further aspects, the IL-7 protein is administered at a dose of 1,460 μg/kg with a dosing frequency of once every 40 days. In certain aspects, the IL-7 protein is administered at a dose of 1,480 μg/kg with a dosing frequency of once every 40 days. In some aspects, the IL-7 protein is administered at a dose of 1,500 μg/kg with a dosing frequency of once every 40 days. In further aspects, the IL-7 protein is administered at a dose of 1,600 μg/kg with a dosing frequency of once every 40 days. In some aspects, the IL-7 protein is administered at a dose of 1,700 μg/kg with a dosing frequency of once every 40 days. In some aspects, the IL-7 protein is administered at a dose of 2,000 μg/kg with a dosing frequency of once every 40 days.

In some aspects, the IL-7 protein is administered at a dose of 60 μg/kg with a dosing frequency of once every 50 days. In some aspects, the IL-7 protein is administered at a dose of 120 μg/kg with a dosing frequency of once every 50 days. In some aspects, the IL-7 protein is administered at a dose of 240 μg/kg with a dosing frequency of once every 50 days. In some aspects, the IL-7 protein is administered at a dose of 480 μg/kg with a dosing frequency of once every 50 days. In some aspects, the IL-7 protein is administered at a dose of 720 μg/kg with a dosing frequency of once every 50 days. In some aspects, the IL-7 protein is administered at a dose of 960 μg/kg with a dosing frequency of once every 50 days. In some aspects, the IL-7 protein is administered at a dose of 1,200 μg/kg with a dosing frequency of once every 50 days. In some aspects, the IL-7 protein is administered at a dose of 1,300 μg/kg with a dosing frequency of once every 50 days. In some aspects, the IL-7 protein is administered at a dose of 1,400 μg/kg with a dosing frequency of once every 50 days. In other aspects, the IL-7 protein is administered at a dose of 1,420 μg/kg with a dosing frequency of once every 50 days. In certain aspects, the IL-7 protein is administered at a dose of 1,440 μg/kg with a dosing frequency of once every 50 days. In further aspects, the IL-7 protein is administered at a dose of 1,460 μg/kg with a dosing frequency of once every 50 days. In certain aspects, the IL-7 protein is administered at a dose of 1,480 μg/kg with a dosing frequency of once every 50 days. In some aspects, the IL-7 protein is administered at a dose of 1,500 μg/kg with a dosing frequency of once every 50 days. In further aspects, the IL-7 protein is administered at a dose of 1,600 μg/kg with a dosing frequency of once every 50 days. In some aspects, the IL-7 protein is administered at a dose of 1,700 μg/kg with a dosing frequency of once every 50 days. In some aspects, the IL-7 protein is administered at a dose of 2,000 μg/kg with a dosing frequency of once every 50 days.

In some aspects, the IL-7 protein is administered at a dose of 60 μg/kg with a dosing frequency of once every 60 days. In some aspects, the IL-7 protein is administered at a dose of 120 μg/kg with a dosing frequency of once every 60 days. In some aspects, the IL-7 protein is administered at a dose of 240 μg/kg with a dosing frequency of once every 60 days. In some aspects, the IL-7 protein is administered at a dose of 480 μg/kg with a dosing frequency of once every 60 days. In some aspects, the IL-7 protein is administered at a dose of 720 μg/kg with a dosing frequency of once every 60 days. In some aspects, the IL-7 protein is administered at a dose of 960 μg/kg with a dosing frequency of once every 60 days. In some aspects, the IL-7 protein is administered at a dose of 1,200 μg/kg with a dosing frequency of once every 60 days. In some aspects, the IL-7 protein is administered at a dose of 1,300 μg/kg with a dosing frequency of once every 60 days. In some aspects, the IL-7 protein is administered at a dose of 1,400 μg/kg with a dosing frequency of once every 60 days. In other aspects, the IL-7 protein is administered at a dose of 1,420 μg/kg with a dosing frequency of once every 60 days. In certain aspects, the IL-7 protein is administered at a dose of 1,440 μg/kg with a dosing frequency of once every 60 days. In further aspects, the IL-7 protein is administered at a dose of 1,460 μg/kg with a dosing frequency of once every 60 days. In certain aspects, the IL-7 protein is administered at a dose of 1,480 μg/kg with a dosing frequency of once every 60 days. In some aspects, the IL-7 protein is administered at a dose of 1,500 μg/kg with a dosing frequency of once every 60 days. In further aspects, the IL-7 protein is administered at a dose of 1,600 μg/kg with a dosing frequency of once every 60 days. In some aspects, the IL-7 protein is administered at a dose of 1,700 μg/kg with a dosing frequency of once every 60 days. In some aspects, the IL-7 protein is administered at a dose of 2,000 μg/kg with a dosing frequency of once every 60 days.

In some aspects, the IL-7 protein is administered at a dose of 60 _{K}g/kg with a dosing frequency of once every 70 days. In some aspects, the IL-7 protein is administered at a dose of 120 μg/kg with a dosing frequency of once every 70 days. In some aspects, the IL-7 protein is administered at a dose of 240 μg/kg with a dosing frequency of once every 70 days. In some aspects, the IL-7 protein is administered at a dose of 480 μg/kg with a dosing frequency of once every 70 days. In some aspects, the IL-7 protein is administered at a dose of 720 μg/kg with a dosing frequency of once every 70 days. In some aspects, the IL-7 protein is administered at a dose of 960 μg/kg with a dosing frequency of once every 70 days. In some aspects, the IL-7 protein is administered at a dose of 1,200 μg/kg with a dosing frequency of once every 70 days. In some aspects, the IL-7 protein is administered at a dose of 1,300 μg/kg with a dosing frequency of once every 70 days. In some aspects, the IL-7 protein is administered at a dose of 1,400 μg/kg with a dosing frequency of once every 70 days. In other aspects, the IL-7 protein is administered at a dose of 1,420 μg/kg with a dosing frequency of once every 70 days. In certain aspects, the IL-7 protein is administered at a dose of 1,440 μg/kg with a dosing frequency of once every 70 days. In further aspects, the IL-7 protein is administered at a dose of 1,460 μg/kg with a dosing frequency of once every 70 days. In certain aspects, the IL-7 protein is administered at a dose of 1,480 μg/kg with a dosing frequency of once every 70 days. In some aspects, the IL-7 protein is administered at a dose of 1,500 μg/kg with a dosing frequency of once every 70 days. In further aspects, the IL-7 protein is administered at a dose of 1,600 μg/kg with a dosing frequency of once every 70 days. In some aspects, the IL-7 protein is administered at a dose of 1,700 μg/kg with a dosing frequency of once every 70 days. In some aspects, the IL-7 protein is administered at a dose of 2,000 μg/kg with a dosing frequency of once every 70 days.

In some aspects, the IL-7 protein is administered at a dose of 60 μg/kg with a dosing frequency of once every 80 days. In some aspects, the IL-7 protein is administered at a dose of 120 μg/kg with a dosing frequency of once every 80 days. In some aspects, the IL-7 protein is administered at a dose of 240 μg/kg with a dosing frequency of once every 80 days. In some aspects, the IL-7 protein is administered at a dose of 480 μg/kg with a dosing frequency of once every 80 days. In some aspects, the IL-7 protein is administered at a dose of 720 μg/kg with a dosing frequency of once every 80 days. In some aspects, the IL-7 protein is administered at a dose of 960 μg/kg with a dosing frequency of once every 80 days. In some aspects, the IL-7 protein is administered at a dose of 1,200 μg/kg with a dosing frequency of once every 80 days. In some aspects, the IL-7 protein is administered at a dose of 1,300 μg/kg with a dosing frequency of once every 80 days. In some aspects, the IL-7 protein is administered at a dose of 1,400 μg/kg with a dosing frequency of once every 80 days. In other aspects, the IL-7 protein is administered at a dose of 1,420 μg/kg with a dosing frequency of once every 80 days. In certain aspects, the IL-7 protein is administered at a dose of 1,440 μg/kg with a dosing frequency of once every 80 days. In further aspects, the IL-7 protein is administered at a dose of 1,460 μg/kg with a dosing frequency of once every 80 days. In certain aspects, the IL-7 protein is administered at a dose of 1,480 μg/kg with a dosing frequency of once every 80 days. In some aspects, the IL-7 protein is administered at a dose of 1,500 μg/kg with a dosing frequency of once every 80 days. In further aspects, the IL-7 protein is administered at a dose of 1,600 μg/kg with a dosing frequency of once every 80 days. In some aspects, the IL-7 protein is administered at a dose of 1,700 μg/kg with a dosing frequency of once every 80 days. In some aspects, the IL-7 protein is administered at a dose of 2,000 μg/kg with a dosing frequency of once every 80 days.

In some aspects, the IL-7 protein is administered at a dose of 60 μg/kg with a dosing frequency of once every 90 days. In some aspects, the IL-7 protein is administered at a dose of 120 μg/kg with a dosing frequency of once every 90 days. In some aspects, the IL-7 protein is administered at a dose of 240 μg/kg with a dosing frequency of once every 90 days. In some aspects, the IL-7 protein is administered at a dose of 480 μg/kg with a dosing frequency of once every 90 days. In some aspects, the IL-7 protein is administered at a dose of 720 μg/kg with a dosing frequency of once every 90 days. In some aspects, the IL-7 protein is administered at a dose of 960 μg/kg with a dosing frequency of once every 90 days. In some aspects, the IL-7 protein is administered at a dose of 1,200 μg/kg with a dosing frequency of once every 90 days. In some aspects, the IL-7 protein is administered at a dose of 1,300 μg/kg with a dosing frequency of once every 90 days. In some aspects, the IL-7 protein is administered at a dose of 1,400 μg/kg with a dosing frequency of once every 90 days. In other aspects, the IL-7 protein is administered at a dose of 1,420 μg/kg with a dosing frequency of once every 90 days. In certain aspects, the IL-7 protein is administered at a dose of 1,440 μg/kg with a dosing frequency of once every 90 days. In further aspects, the IL-7 protein is administered at a dose of 1,460 μg/kg with a dosing frequency of once every 90 days. In certain aspects, the IL-7 protein is administered at a dose of 1,480 μg/kg with a dosing frequency of once every 90 days. In some aspects, the IL-7 protein is administered at a dose of 1,500 μg/kg with a dosing frequency of once every 90 days. In further aspects, the IL-7 protein is administered at a dose of 1,600 μg/kg with a dosing frequency of once every 90 days. In some aspects, the IL-7 protein is administered at a dose of 1,700 μg/kg with a dosing frequency of once every 90 days. In some aspects, the IL-7 protein is administered at a dose of 2,000 μg/kg with a dosing frequency of once every 90 days.

In some aspects, the IL-7 protein is administered at a dose of 60 μg/kg with a dosing frequency of once every 100 days. In some aspects, the IL-7 protein is administered at a dose of 120 μg/kg with a dosing frequency of once every 100 days. In some aspects, the IL-7 protein is administered at a dose of 240 μg/kg with a dosing frequency of once every 100 days. In some aspects, the IL-7 protein is administered at a dose of 480 μg/kg with a dosing frequency of once every 100 days. In some aspects, the IL-7 protein is administered at a dose of 720 μg/kg with a dosing frequency of once every 100 days. In some aspects, the IL-7 protein is administered at a dose of 960 μg/kg with a dosing frequency of once every 100 days. In some aspects, the IL-7 protein is administered at a dose of 1,200 μg/kg with a dosing frequency of once every 100 days. In some aspects, the IL-7 protein is administered at a dose of 1,300 μg/kg with a dosing frequency of once every 100 days. In some aspects, the IL-7 protein is administered at a dose of 1,400 μg/kg with a dosing frequency of once every 100 days. In other aspects, the IL-7 protein is administered at a dose of 1,420 μg/kg with a dosing frequency of once every 100 days. In certain aspects, the IL-7 protein is administered at a dose of 1,440 μg/kg with a dosing frequency of once every 100 days. In further aspects, the IL-7 protein is administered at a dose of 1,460 μg/kg with a dosing frequency of once every 100 days. In certain aspects, the IL-7 protein is administered at a dose of 1,480 μg/kg with a dosing frequency of once every 100 days. In some aspects, the IL-7 protein is administered at a dose of 1,500 μg/kg with a dosing frequency of once every 100 days. In further aspects, the IL-7 protein is administered at a dose of 1,600 μg/kg with a dosing frequency of once every 100 days. In some aspects, the IL-7 protein is administered at a dose of 1,700 μg/kg with a dosing frequency of once every 100 days. In some aspects, the IL-7 protein is administered at a dose of 2,000 μg/kg with a dosing frequency of once every 100 days.

In some aspects, methods disclosed herein (e.g., administering an IL-7 protein in combination with a bispecific antibody) can be used in combination with one or more additional anti-cancer and/or immunomodulating agents. Such agents can include, for example, chemotherapy drugs, small molecule drugs, or antibodies that stimulate the immune response to a given cancer. In some aspects, the methods described herein are used in combination with a standard of care treatment (e.g., surgery, radiation, and chemotherapy). Methods described herein can also be used as a maintenance therapy, e.g., a therapy that is intended to prevent the occurrence or recurrence of tumors.

In some aspects, a method for treating a tumor disclosed herein can comprise administering an IL-7 protein and a bispecific antibody in combination with one or more additional immuno-oncology agents, such that multiple elements of the immune pathway can be targeted. Non-limiting of such combinations include: a therapy that enhances tumor antigen presentation (e.g., dendritic cell vaccine, GM-CSF secreting cellular vaccines, CpG oligonucleotides, imiquimod); a therapy that inhibits negative immune regulation e.g., by inhibiting CTLA-4 and/or PD1/PD-L1/PD-L2 pathway and/or depleting or blocking Tregs or other immune suppressing cells (e.g., myeloid-derived suppressor cells); a therapy that stimulates positive immune regulation, e.g., with agonists that stimulate the CD-137, OX-40, and/or CD40 or GITR pathway and/or stimulate T cell effector function; a therapy that increases systemically the frequency of anti-tumor T cells; a therapy that depletes or inhibits Tregs, such as Tregs in the tumor, e.g., using an antagonist of CD25 (e.g., daclizumab) or by ex vivo anti-CD25 bead depletion; a therapy that impacts the function of suppressor myeloid cells in the tumor; a therapy that enhances immunogenicity of tumor cells (e.g., anthracyclines); adoptive T cell or NK cell transfer including genetically modified cells, e.g., cells modified by chimeric antigen receptors (CAR-T therapy); a therapy that inhibits a metabolic enzyme such as indoleamine dioxigenase (IDO), dioxigenase, arginase, or nitric oxide synthetase; a therapy that reverses/prevents T cell anergy or exhaustion; a therapy that triggers an innate immune activation and/or inflammation at a tumor site; administration of immune stimulatory cytokines; or blocking of immuno repressive cytokines.

In some aspects, an immuno-oncology agent that can be used with the present disclosure comprises an immune checkpoint inhibitor (i.e., blocks signaling through the particular immune checkpoint pathway). Non-limiting examples of immune checkpoint inhibitors that can be used in the present methods comprise a CTLA-4 antagonist (e.g., anti-CTLA-4 antibody), PD-1 antagonist (e.g., anti-PD-1 antibody, anti-PD-L1 antibody), TIM-3 antagonist (e.g., anti-TIM-3 antibody), or combinations thereof.

In some aspects, an immuno-oncology agent comprises an immune checkpoint activator (i.e., promotes signaling through the particular immune checkpoint pathway). In certain aspects, immune checkpoint activator comprises OX40 agonist (e.g., anti-OX40 antibody), LAG-3 agonist (e.g. anti-LAG-3 antibody), 4-1BB (CD137) agonist (e.g., anti-CD137 antibody), GITR agonist (e.g., anti-GITR antibody), or any combination thereof.

In some aspects, a combination of an IL-7 protein and a second agent discussed herein (e.g., bispecific antibody) can be administered concurrently as a single composition in a pharmaceutically acceptable carrier. In other aspects, a combination of an IL-7 protein and a second agent discussed herein (e.g., bispecific antibody) can be administered concurrently as separate compositions. In further aspects, a combination of an IL-7 protein and a second agent discussed herein (e.g., bispecific antibody) can be administered sequentially. In some aspects, an IL-7 protein is administered prior to the administration of a second agent (e.g., bispecific antibody). In other aspects, an IL-7 protein is administered after the administration of a second agent (e.g., bispecific antibody).

IIIa. IL-7 Proteins Useful for the Disclosure

Disclosed herein are IL-7 proteins that can be used in combination with a bispecific antibody to treat a cancer (or a tumor). In some aspects, IL-7 protein useful for the present uses can be wild-type IL-7 or modified IL-7 (i.e., not wild-type IL-7 protein) (e.g., IL-7 variant, IL-7 functional fragment, IL-7 derivative, or any combination thereof, e.g., fusion protein, chimeric protein, etc.) as long as the IL-7 protein contains one or more biological activities of IL-7, e.g., capable of binding to IL-7R, e.g., inducing early T-cell development, promoting T-cell homeostasis. See ElKassar and Gress. J. Immunotoxicol. 2010 March; 7(1): 1-7. In some aspects, an IL-7 protein of the present disclosure is not a wild-type IL-7 protein (i.e., comprises one or more modifications). Non-limiting examples of such modifications can include an oligopeptide and/or a half-life extending moiety. See WO 2016/200219, which is herein incorporated by reference in its entirety.

IL-7 binds to its receptor which is composed of the two chains IL-7Rα (CD127), shared with the thymic stromal lymphopoietin (TSLP) (Ziegler and Liu, 2006), and the common γ chain (CD132) for IL-2, IL-15, IL-9 and IL-21. Whereas γc is expressed by most hematopoietic cells, IL-7Ra is nearly exclusively expressed on lymphoid cells. After binding to its receptor, IL-7 signals through two different pathways: Jak-Stat (Janus kinase-Signal transducer and activator of transcription) and PI3K/Akt responsible for differentiation and survival, respectively. The absence of IL-7 signaling is responsible for a reduced thymic cellularity as observed in mice that have received an anti-IL-7 neutralizing monoclonal antibody (MAb); Grabstein et al., 1993), in IL-7−/− (von Freeden-Jeffry et al., 1995), IL-7Rα−/− (Peschon et al., 1994; Maki et al., 1996), γc−/− (Malissen et al., 1997), and Jak3−/− mice (Park et al., 1995). In the absence of IL-7 signaling, mice lack T-, B-, and NK-T cells. IL-7a−/− mice (Peschon et al., 1994) have a similar but more severe phenotype than IL-7−/− mice (von Freeden-Jeffry et al., 1995), possibly because TSLP signaling is also abrogated in IL-7α−/− mice. IL-7 is required for the development of γδ cells (Maki et al., 1996) and NK-T cells (Boesteanu et al., 1997).

In some aspects, an IL-7 protein useful for the present disclosure comprises an amino acid sequence as set forth in any one of SEQ ID NOs: 1 to 6. In other aspects, the IL-7 protein comprises an amino acid sequence having a sequence identity of about 70%, about 75%, about 80%, about 85%, about 90%, about 91%, about 92%, about 93%, about 94%, about 95%, about 96%, about 97%, about 98%, or about 99% or higher, to a sequence of SEQ ID NOS: 1 to 6.

In some aspects, the IL-7 protein includes a modified IL-7 or a fragment thereof, wherein the modified IL-7 or the fragment retains one or more biological activities of wild-type IL-7. In some aspects, the IL-7 protein can be derived from humans, rats, mice, monkeys, cows, or sheep.

In some aspects, the human IL-7 can have an amino acid sequence represented by SEQ ID NO: 1 (Genbank Accession No. P13232); the rat IL-7 can have an amino acid sequence represented by SEQ ID NO: 2 (Genbank Accession No. P56478); the mouse IL-7 can have an amino acid sequence represented by SEQ ID NO: 3 (Genbank Accession No. P10168); the monkey IL-7 may have an amino acid sequence represented by SEQ ID NO: 4 (Genbank Accession No. NP 001279008); the cow IL-7 can have an amino acid sequence represented by SEQ ID NO: 5 (Genbank Accession No. P26895), and the sheep IL-7 can have an amino acid sequence represented by SEQ ID NO: 6 (Genbank Accession No. Q28540).

In some aspects, an IL-7 protein useful for the present disclosure comprises an IL-7 fusion protein. In certain aspects, an IL-7 fusion protein comprises (i) an oligopeptide and (i) an IL-7 or a variant thereof. In some aspects, the oligopeptide is linked to the N-terminal region of the IL-7 or a variant thereof

In some aspects, an oligopeptide disclosed herein consists of 1 to 10 amino acids.

In certain aspects, an oligopeptide consists of at least 1, at least 2, at least 3, at least 4, at least 5, at least 6, at least 7, at least 8, at least 9, or 10 amino acids. In some aspects, one or more amino acids of an oligopeptide are selected from the group consisting of methionine, glycine, and combinations thereof In certain aspects, an oligopeptide is selected from the group consisting of methionine (M), glycine (G), methionine-methionine (MM), glycine-glycine (GG), methionine-glycine (MG), glycine-methionine (GM), methionine-methionine-methionine (MMM), methionine-methionine-glycine (MMG), methionine-glycine-methionine (MGM), glycine-methionine-methionine (GMM), methionine-glycine-glycine (MGG), glycine-methionine-glycine (GMG), glycine-glycine-methionine (GGM), glycine-glycine-glycine (GGG), methionine-glycine-glycine-methionine (MGGM) (SEQ ID NO: 41), methionine-methionine-glycine-glycine (MMGG) (SEQ ID NO: 42), glycine-glycine-methionine-methionine (GGMM) (SEQ ID NO: 43), methionine-glycine-methionine-glycine (MGMG) (SEQ ID NO: 44), glycine-methionine-methionine-glycine (GMMG) (SEQ ID NO: 45), glycine-glycine-glycine-methionine (GGGM) (SEQ ID NO: 46), methionine-glycine-glycine-glycine (MGGG) (SEQ ID NO: 47), glycine-methionine-glycine-glycine (GMGG) (SEQ ID NO: 48), glycine-glycine-methionine-glycine (GGMG) (SEQ ID NO: 49), glycine-glycine-methionine-methionine-methionine (GGMMM) (SEQ ID NO: 50), glycine-glycine-glycine-methionine-methionine (GGGMM) (SEQ ID NO: 51), glycine-glycine-glycine-glycine-methionine (GGGGM) (SEQ ID NO: 52), methionine-glycine-methionine-methionine-methionine (MGMMM) (SEQ ID NO: 53), methionine-glycine-glycine-methionine-methionine (MGGMM) (SEQ ID NO: 54), methionine-glycine-glycine-glycine-methionine (MGGGM) (SEQ ID NO: 55), methionine-methionine-glycine-methionine-methionine (MMGMM) (SEQ ID NO: 56), methionine-methionine-glycine-glycine-methionine (MMGGM) (SEQ ID NO: 57), methionine-methionine-glycine-glycine-glycine (MMGGG) (SEQ ID NO: 58), methionine-methionine-methionine-glycine-methionine (MMMGM) (SEQ ID NO: 59), methionine-glycine-methionine-glycine-methionine (MGMGM) (SEQ ID NO: 60), glycine-methionine-glycine-methionine-glycine (GMGMG) (SEQ ID NO: 61), glycine-methionine-methionine-methionine-glycine (GMGMG) (SEQ ID NO: 62), glycine-glycine-methionine-glycine-methionine (GGMGM) (SEQ ID NO: 63), glycine-glycine-methionine-methionine-glycine (GGMMG) (SEQ ID NO: 64), glycine-methionine-methionine-glycine-methionine (GMMGM) (SEQ ID NO: 65), methionine-glycine-methionine-methionine-glycine (MGMMG) (SEQ ID NO: 66), glycine-methionine-glycine-glycine-methionine (GMGGM) (SEQ ID NO: 67), methionine-methionine-glycine-methionine-glycine (MMGMG) (SEQ ID NO: 68), glycine-methionine-methionine-glycine-glycine (GMMGG) (SEQ ID NO: 69), glycine-methionine-glycine-glycine-glycine (GMGGG) (SEQ ID NO: 70), glycine-glycine-methionine-glycine-glycine (GGMGG) (SEQ ID NO: 71), glycine-glycine-glycine-glycine-glycine (GGGGG) (SEQ ID NO: 72), or combinations thereof. In some aspects, an oligopeptide is methionine-glycine-methionine (MGM).

In some aspects, an IL-7 fusion protein comprises (i) an IL-7 or a variant thereof, and (ii) a half-life extending moiety. In some aspects, a half-life extending moiety extends the half-life of the IL-7 or variant thereof. In some aspects, a half-life extending moiety is linked to the C-terminal region of an IL-7 or a variant thereof.

In some aspects, an IL-7 fusion protein comprises (i) IL-7 (a first domain), (ii) a second domain that includes an amino acid sequence having 1 to 10 amino acid residues consisting of methionine, glycine, or a combination thereof, e.g., MGM, and (iii) a third domain comprising a half-life extending moiety. In some aspects, the half-life extending moiety can be linked to the N-terminal or the C-terminal of the first domain or the second domain. Additionally, the IL-7 including the first domain and the second domain can be linked to both terminals of the third domain.

Non-limiting examples of half-life extending moieties include: Fc, albumin, an albumin-binding polypeptide, Pro/Ala/Ser (PAS), a C-terminal peptide (CTP) of the f3 subunit of human chorionic gonadotropin, polyethylene glycol (PEG), long unstructured hydrophilic sequences of amino acids (XTEN), hydroxyethyl starch (HES), an albumin-binding small molecule, and combinations thereof.

In some aspects, a half-life extending moiety is Fc. In certain aspects, Fc is from a modified immunoglobulin in which the antibody-dependent cellular cytotoxicity (ADCC) or complement-dependent cytotoxicity (CDC) weakened due to the modification in the binding affinity with the Fc receptor and/or a complement. In some aspects, the modified immunoglobulin can be selected from the group consisting of IgG1, IgG2, IgG3, IgG4, IgA1, IgA2, IgD, IgE, and a combination thereof. In some aspects, an Fc is a hybrid Fc (“hFc” or “hyFc”), comprising a hinge region, a CH2 domain, and a CH3 domain. In certain aspects, a hinge region of a hybrid Fc disclosed herein comprises a human IgD hinge region. In certain aspects, a CH2 domain of a hybrid Fc comprises a part of human IgD CH2 domain and a part of human IgG4 CH2 domain. In certain aspects, a CH3 domain of a hybrid Fc comprises a part of human IgG4 CH3 domain. Accordingly, in some aspects, a hybrid Fc disclosed herein comprises a hinge region, a CH2 domain, and a CH3 domain, wherein the hinge region comprises a human IgD hinge region, wherein the CH2 domain comprises a part of human IgD CH2 domain and a part of human IgG4 CH2 domain, and wherein the CH3 domain comprises a part of human IgG4 CH3 domain.

In some aspects, an Fc disclosed herein can be an Fc variant. As used herein, the term “Fc variant” refers to an Fc which was prepared by substituting a part of the amino acids among the Fc region or by combining the Fc regions of different kinds. The Fc region variant can prevent from being cut off at the hinge region. Specifically, in some aspects, a Fc variant comprises modifications at the 144^{th} amino acid and/or 145^{th} amino acid of SEQ ID NO: 9. In certain aspects, the 144^{th} amino acid (K) and/or the 145^{th} amino acid (K) is substituted with G or S.

In some aspects, an Fc or an Fc variant disclosed herein can be represented by the following formula: N′-(Z1)p-Y-Z2-Z3-Z4-C, wherein:

N comprises the N-terminus;

Z1 comprises an amino acid sequence having 5 to 9 consecutive amino acid residues from the amino acid residue at position 98 toward the N-terminal, among the amino acid residues at positions from 90 to 98 of SEQ ID NO: 7;

Y comprises an amino acid sequence having 5 to 64 consecutive amino acid residues from the amino acid residue at position 162 toward the N-terminal, among the amino acid residues at positions from 99 to 162 of SEQ ID NO: 7;

Z2 comprises an amino acid sequence having 4 to 37 consecutive amino acid residues from the amino acid residue at position 163 toward the C-terminal, among the amino acid residues at positions from 163 to 199 of SEQ ID NO: 7;

Z3 comprises an amino acid sequence having 71 to 106 consecutive amino acid residues from the amino acid residue at position 220 toward the N-terminal, among the amino acid residues at positions from 115 to 220 of SEQ ID NO: 8; and

Z4 comprises an amino acid sequence having 80 to 107 consecutive amino acid residues from the amino acid residue at position 221 toward the C-terminal, among the amino acid residues at positions from 221 to 327 of SEQ ID NO: 8.

In some aspects, a Fc region disclosed herein can include the amino acid sequence of SEQ ID NO: 9 (hyFc), SEQ ID NO: 10 (hyFcM1), SEQ ID NO: 11 (hyFcM2), SEQ ID NO: 12 (hyFcM3), or SEQ ID NO: 13 (hyFcM4). In some aspects, the Fe region can include the amino acid sequence of SEQ ID NO: 14 (a non-lytic mouse Fc).

Other non-limiting examples of Fc regions that can be used with the present disclosure are described in U.S. Pat. No. 7,867,491, which is herein incorporated by reference in its entirety.

In some aspects, an IL-7 fusion protein disclosed herein comprises both an oligopeptide and a half-life extending moiety.

In some aspects, an IL-7 protein can be fused to albumin, a variant, or a fragment thereof. Examples of the IL-7-albumin fusion protein can be found at International Application Publication No. WO 2011/124718 A1. In some aspects, an IL-7 protein is fused to a pre-pro-B cell Growth Stimulating Factor (PPBSF), optionally by a flexible linker. See US 2002/0058791A1. In other aspects, an IL-7 protein useful for the disclosure is an IL-7 conformer that has a particular three dimensional structure. See US 2005/0249701 A1. In some aspects, an IL-7 protein can be fused to an Ig chain, wherein amino acid residues 70 and 91 in the IL-7 protein are glycosylated the amino acid residue 116 in the IL-7 protein is non-glycosylated. See U.S. Pat. No. 7,323,549 B2. In some aspects, an IL-7 protein that does not contain potential T-cell epitopes (thereby to reduce anti-IL-7 T-cell responses) can also be used for the present disclosure. See US 2006/0141581 A1. In other aspects, an IL-7 protein that has one or more amino acid residue mutations in carboxy-terminal helix D region can be used for the present disclosure. The IL-7 mutant can act as IL-7R partial agonist despite lower binding affinity for the receptor. See US 2005/0054054A1. Any IL-7 proteins described in the above listed patents or publications are incorporated herein by reference in their entireties.

In addition, non-limiting examples of additional IL-7 proteins useful for the present disclosure are described in U.S. Pat. Nos. 7,708,985, 8,034,327, 8,153,114, 7,589,179, 7,323,549, 7,960,514, 8,338,575, 7,118,754, 7,488,482, 7,670,607, 6,730,512, WO0017362, GB2434578A, WO 2010/020766 A2, WO91/01143, Beq et al., Blood, vol. 114 (4), 816, 23 Jul. 2009, Kang et al., J. Virol. Doi:10.1128/JVI.02768-15, Martin et al., Blood, vol. 121 (22), 4484, May 30, 2013, McBride et al., Acta Oncologica, 34:3, 447-451, Jul. 8, 2009, and Xu et al., Cancer Science, 109: 279-288, 2018, which are incorporated herein by reference in their entireties.

The present disclosure is directed to a method for treating a tumor (or a cancer) in a subject in need thereof, comprising administering to the subject an effective amount of an interleukin-7 (IL-7) protein in combination with an effective amount of an immune checkpoint inhibitor. Non-limiting examples of immune checkpoint inhibitors that can be used with the current methods include an anti-PD-1 antibody, anti-PD-L1 antibody, anti-CTLA-4 antibody, and combinations thereof.

In some aspects, an oligopeptide disclosed herein is directly linked to the N-terminal region of IL-7 or a variant thereof. In other aspects, an oligopeptide is linked to the N-terminal region via a linker. In some aspects, a half-life extending moiety disclosed herein is directly linked to the C-terminal region of IL-7 or a variant thereof. In certain aspects, a half-life extending moiety is linked to the C-terminal region via a linker. In some aspects, a linker is a peptide linker. In certain aspects, a peptide linker comprises a peptide of 10 to 20 amino acid residues consisting of Gly and Ser residues. In some aspects, a linker is an albumin linker. In some aspects, a linker is a chemical bond. In certain aspects, a chemical bond comprises a disulfide bond, a diamine bond, a sulfide-amine bond, a carboxy-amine bond, an ester bond, a covalent bond, or combinations thereof. When the linker is a peptide linker, in some aspects, the connection can occur in any linking region. They may be coupled using a crosslinking agent known in the art. In some aspects, examples of the crosslinking agent can include N-hydroxy succinimide esters such as 1,1-bis(diazoacetyl)-2-phenylethane, glutaraldehyde, and 4-azidosalicylic acid; imido esters including disuccinimidyl esters such as 3,3′-dithiobis (succinimidyl propionate), and bifunctional maleimides such as bis-Nmaleimido-1,8-octane, but is not limited thereto.

In some aspects, an IL-7 (or variant thereof) portion of IL-7 fusion protein disclosed herein comprises an amino sequence that is at least 70%, at least 75%, at least 80%, at least 85%, at least 90%, at least 91%, at least 92%, at least 93%, at least 94%, at least 95%, at least 96%, at least 97%, at least 98%, at least 98%, or at least 99% identical to an amino acid sequence set forth in SEQ ID NOs: 15-20. In certain aspects, an IL-7 (or variant thereof) portion of IL-7 fusion protein disclosed herein comprises the amino acid sequence set forth in SEQ ID NOs: 15-20.

In some aspects, an IL-7 fusion protein comprises: a first domain including a polypeptide having the activity of IL-7 or a similar activity thereof; a second domain comprising an amino acid sequence havingl to 10 amino acid residues consisting of methionine, glycine, or a combination thereof; and a third domain, which is an Fc region of modified immunoglobulin, coupled to the C-terminal of the first domain.

In some aspects, an IL-7 fusion protein that can be used with the present methods comprises an amino sequence that is at least 70%, at least 75%, at least 80%, at least 85%, at least 90%, at least 91%, at least 92%, at least 93%, at least 94%, at least 95%, at least 96%, at least 97%, at least 98%, at least 98%, or at least 99% identical to an amino acid sequence set forth in SEQ ID NOs: 21-25. In certain aspects, an IL-7 fusion protein of the present disclosure comprises the amino acid sequence set forth in SEQ ID NOs: 21-25. In further aspects, an IL-7 fusion protein disclosed herein comprises the amino acid sequence set forth in SEQ ID NOs: 26 and 27.

In some aspects, an IL-7 protein useful for the present disclosure can increase absolute lymphocyte counts in a subject when administered to the subject. In certain aspects, the subject suffers from a disease or disorder described herein (e.g., cancer). In other aspects, the subject is a healthy individual (e.g., does not suffer from a disease or disorder described herein, e.g., cancer). In certain aspects, the absolute lymphocyte count is increased by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or about 100% or more, compared to a reference (e.g., corresponding level in a subject that did not receive the IL-7 protein).

In some aspects, an IL-7 protein disclosed herein can increase T cell proliferation (e.g., CD8⁺ T cells) in a subject. In certain aspects, the increase in T cell proliferation occurs in the periphery (e.g., not within the tumor). In certain aspects, T cell proliferation is increased by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or about 100% or more, compared to a reference (e.g., corresponding level in a subject that did not receive the IL-7 protein). In certain aspects, T cells (e.g., CD8⁺ T cells) that proliferate in response to the IL-7 administration express one or more of the following markers: Eomesodermin (Eomes), granzyme B, CXCR3, IFN-γ, or combinations thereof.

In some aspects, an IL-7 protein of the present disclosure can increase the recruitment of effector T cells (e.g., cytotoxic CD8⁺ T lymphocytes) to the tumor. In certain aspects, recruitment of effector T cells to the tumor is increased by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or about 100% or more, compared to a reference (e.g., corresponding level in a subject that did not receive the IL-7 protein).

In some aspects, an IL-7 protein of the present disclosure can decrease the number and/or percentage of myeloid-derived suppressor cells (MDSCs) in the tumor of a subject. In certain aspects, the number and/or percentage of MDSCs in the tumor is decreased by at least about 5%, at least about 10%, at least about 20%, at least about 30%, at least about 40%, at least about 50%, at least about 60%, at least about 70%, at least about 80%, at least about 90%, or about 100% compared to a reference (e.g., corresponding level in a subject that did not receive the IL-7 protein).

In some aspects, an IL-7 protein that can be used with the present disclosure can increase the ratio of CD8⁺TILs to MDSCs in a tumor when administered to a subject. In certain aspects, the ratio of CD8⁺ TILs to MDSCs is increased by at least about 5%, at least about 10%, at least about 20%, at least about 30%, at least about 40%, at least about 50%, at least about 60%, at least about 70%, at least about 80%, at least about 90%, at least about 100%, at least about 125%, at least about 150%, at least about 200%, at least about 250%, or at least about 300% after the administration compared to a reference (e.g., corresponding level in a subject that did not receive the IL-7 protein).

IIIb. Bispecific Antibodies

A bispecific antibody that can be used with the present disclosure comprises a first antigen binding moiety and a second antigen binding moiety. In some aspects, each of the first and second antigen binding moieties bind to a distinct (i.e., different) epitope on an antigen. In certain aspects, the first and second antigen binding moieties bind to distinct epitopes on a single antigen (e.g., the bispecific antibody binds to two different regions of a single protein). In other aspects, the first and second antigen binding moieties bind to distinct epitopes on different antigens (e.g., the bispecific antibody binds to an epitope on Protein A and an epitope on Protein B).

In some aspects, the first and/or the second antigen binding moiety is capable of specifically binding to an antigen expressed on an immune cell. As used herein, the term “immune cell” refers to a cell that plays a role in an immune response. In certain aspects, an immune cell comprises a T-lymphocyte (e.g., effector CD4⁺ T-cell or effector CD8⁺ T-cell), B-lymphocyte, natural killer (NK) cells, monocytes, macrophages, eosinophils, mast cells, basophils, granulocytes, dendritic cells, or combinations thereof. In some aspects, an immune cell comprises tumor-infiltrating lymphocytes (TILs), such as CD4+ TILs or CD8+ TILs.

In some aspects, an antigen expressed on an immune cell comprises any molecule expressed on the immune cell. Non-limiting examples of such antigens include CD2, CD3, CD4, CD5, CD8, CD11b, CD14, CD16, CD19, CD28, CD32, CD45, CD56, CD64, KLRG-1, NKG2D, NKp30, DNAM-1, or combinations thereof. In certain aspects, an antigen expressed on an immune cell is CD3. In other aspects, an antigen expressed on an immune cell is NKG2D. In further aspects, an antigen expressed on an immune cell is NKp30. In yet further aspects, an antigen expressed on an immune cell is KLRG-1.

In some aspects, the first and/or the second antigen binding moiety of a bispecific antibody useful for the present disclosure is capable of specifically binding to a tumor antigen. Non-limiting examples of tumor antigens include guanylate cyclase C (GC-C), epidermal growth factor receptor (EGFR or erbB-1), human epidermal growth factor receptor 2 (HER2 or erbB2), erbB-3, erbB-4, MUC-1, melanoma-associated chondroitin sulfate proteoglycan (MCSP), mesothelin (MSLN), folate receptor 1 (FOLR1), CD4, CD19, CD20, CD22, CD30, CD33, CD38, CD44, CD44v6, CD44v7/8, CD70, CD123, CD138, CD171, CEA, CSPG4, CXCRS, c-Met, HERV-envelope protein, eriostin, Bigh3, SPARC, BCR, CD79, CD37, EGFRvIII, EGP2, EGP40, IGFr, L1CAM, AXL, Tissue Factor (TF), CD74, EpCAM, EphA2, MRP3cadherin 19 (CDH19), epidermal growth factor 2 (HER2), 5T4, 8H9, αᵥβ₆ integrin, BCMA, B7-H3, B7-H6, CAIX, CA9, FAP, FBP, fetal AchR, FRcc, GD2, GD3, glypican-1 (GPC1), glypican-2 (GPC2), glypican-3 (GPC3), HLA-A1+MAGE1, HLA-A1+NY-ESO-1, IL-13Rcc2, Lewis-Y, KDR, MCSP, mesothelin, Muc1, Muc16, NCAM, NKG2D ligands, NY-ESO-1, PRAME, PSC1, PSCA, PSMA, ROR1, ROR2, SP17, surviving, TAG72, TEMs, carcinoembryonic antigen, HMW-MAA, VEGF, CLDN18.2, or combinations thereof. In some aspects, a tumor antigen comprises guanylate cyclase C (GC-C).

In some aspects, a tumor antigen comprises an immune checkpoint molecule. In certain aspects, an immune checkpoint molecule comprises a PD-1 ligand (e.g., PD-L1 or PD-L2), LAG3 ligand, TIM-3 ligand (e.g., galectin 9), CTLA-4 ligand (e.g., CD28), OX40 ligand, CD28 ligand (e.g., B7H3 or B7H4), or combinations thereof.

In some aspects, the first antigen binding moiety and the second antigen binding moiety do not target the same antigen. Accordingly, in certain aspects, a bispecific antibody useful for the present disclosure comprises a first antigen binding moiety and a second antigen binding moiety, wherein (i) the first antigen binding moiety specifically binds to an antigen expressed on an immune cell, and the second antigen binding moiety specifically binds to a tumor antigen; or (ii) the first antigen binding moiety specifically binds to a tumor antigen, and the second antigen binding moiety specifically binds to an antigen expressed on an immune cell. In some aspects, one of the antigen binding moieties bind to a PD-L1 ligand (e.g., PD-L1) and the other antigen binding moiety binds to CD3. In certain aspects, an anti-PD-L1/CD3 bispecific antibody that can be used with the present disclosure comprises the amino acid sequence set forth in SEQ ID NO: 73.

In some aspects, a bispecific antibody useful for the present disclosure can include any bispecific antibodies known in the art. In certain aspects, a bispecific antibody comprises a dual-variable-domain antibody (DVD-Ig), where each light chain and heavy chain contains two variable domains in tandem through a short peptide linkage (Wu et al., Generation and Characterization of a Dual Variable Domain Immunoglobulin (DVD-Ig™) Molecule, In: Antibody Engineering, Springer Berlin Heidelberg (2010)). In some aspects, a bispecific antibody is a chemically-linked bispecific (Fab′)2 fragment. In certain aspects, a bispecific antibody comprises a Tandab (i.e., a fusion of two single chain diabodies resulting in a tetravalent bispecific antibody that has two binding sites for each of the target antigens). In further aspects, a bispecific antibody is a flexibody, which is a combination of scFvs with a diabody resulting in a multivalent molecule. In still further aspects, a bispecific antibody comprises a “dock and lock” molecule, based on the “dimerization and docking domain” in Protein Kinase A, which, when applied to Fabs, can yield a trivalent bispecific binding protein consisting of two identical Fab fragments linked to a different Fab fragment. In some aspects, a bispecific antibody comprises a “Scorpion molecule,” comprising, e.g., two scFvs fused to both termini of a human Fab-arm. In certain aspects, a bispecific antibody comprises a diabody.

In some aspects, classes of bispecific antibodies include but are not limited to IgG-like molecules with complementary CH3 domains to force heterodimerization; IgG fusion molecules, wherein full length IgG antibodies are fused to extra Fab fragment or parts of Fab fragment; Fc fusion molecules, wherein single chain Fv molecules or stabilized diabodies are fused to heavy-chain constant-domains, Fc-regions or parts thereof; Fab fusion molecules, wherein different Fab-fragments are fused together; recombinant IgG-like dual targeting molecules, wherein the two sides of the molecule each contain the Fab fragment or part of the Fab fragment of at least two different antibodies; scFv- and diabody-based and heavy chain antibodies (e.g., domain antibodies, nanobodies) wherein different single chain Fv molecules or different diabodies or different heavy-chain antibodies (e.g. domain antibodies, nanobodies) are fused to each other or to another protein or carrier molecule.

Examples of Fab fusion bispecific antibodies include but are not limited to F(ab)2 (Medarex/AMGEN), Dual-Action or Bis-Fab (Genentech), Dock-and-Lock (DNL) (ImmunoMedics), Bivalent Bispecific (Biotecnol) and Fab-Fv (UCB-Celltech).

Examples of scFv-, diabody-based and domain antibodies include but are not limited to bispecific T Cell Engager (BiTE) (Micromet), tandem Diabody (Tandab) (Affimed), dual Affinity Retargeting Technology (DART) (MacroGenics), single-chain Diabody (Academic), TCR-like Antibodies (AIT, ReceptorLogics), human serum albumin ScFv fusion (Merrimack) and COMBODY® (Epigen Biotech), dual targeting nanobodies (Ablynx), and dual targeting heavy chain only domain antibodies.

In some aspects, a bispecific antibody useful for the present disclosure comprises a Fc region with one or more modifications (e.g., by amino acid substitution, deletion and/or insertion), wherein the modifications one or more functional properties of the antibody (e.g., Fc receptor binding, inflammatory cytokine release, serum half-life, complement fixation, and/or antigen-dependent cellular cytotoxicity). In some aspects, a bispecific antibody can be chemically modified (e.g., one or more chemical moieties can be attached to the antibody) or be modified to alter its glycosylation, to alter one or more functional properties of the antibody. Examples of such modifications are known in the art. See, e.g., U.S. Publ. No. 2018/0371086 A1; U.S. Pat. No. 2015/0210763 A1; and U.S. Pat. No. 9,505,826 B2, each of which is incorporated herein by its entirety.

In some aspects, a bispecific antibody that can be used with the present disclosure can be linked to a therapeutic agent to form an immunoconjugate, such as an antibody-drug conjugate (ADC). Suitable therapeutic agents include antimetabolites, alkylating agents, DNA minor groove binders, DNA intercalators, DNA crosslinkers, histone deacetylase inhibitors, nuclear export inhibitors, proteasome inhibitors, topoisomerase I or II inhibitors, heat shock protein inhibitors, tyrosine kinase inhibitors, antibiotics, anti-mitotic agents, or other anti-cancer agents. ADCs can be prepared as described in U.S. Pat. Nos. 7,087,600; 6,989,452; and 7,129,261; PCT Publication Nos. WO 02/096910; WO 07/038658; WO 07/051081; WO 07/059404; WO 08/083312; and WO 08/103693; U.S. Patent Publication Nos. 20060024317; 20060004081; and 20060247295, each of which is incorporated herein by its entirety. Other examples of agents that can be linked to a bispecific antibody include macromolecular substances such as polymers (e.g., polyethylene glycol (PEG), polyethylenimine (PEI) modified with PEG (PEI-PEG), polyglutamic acid (PGA) (N-(2-Hydroxypropyl) methacrylamide (HPMA) copolymers), hyaluronic acid, radioactive materials (e.g., ⁹⁰Y, ¹³¹I, ¹²⁵I, ³⁵S, ³H, ¹²¹In, ⁹⁹TC), fluorescent substances (e.g., fluorescein and rhodamine), luminescent substances (e.g., luminol), haptens, enzymes (e.g., glucose oxidase), metal chelates, biotin, avidin, and drugs.

### IV. Nucleic Acids, Vectors, Host Cells

Further aspect described herein pertains to one or more nucleic acid molecules that encode a therapeutic agent described herein (e.g., an IL-7 protein). The nucleic acids can be present in whole cells, in a cell lysate, or in a partially purified or substantially pure form. A nucleic acid is “isolated” or “rendered substantially pure” when purified away from other cellular components or other contaminants, e.g., other cellular nucleic acids (e.g., other chromosomal DNA, e.g., the chromosomal DNA that is linked to the isolated DNA in nature) or proteins, by standard techniques, including alkaline/SDS treatment, CsCl banding, column chromatography, restriction enzymes, agarose gel electrophoresis and others well known in the art. See, F. Ausubel, et al. , ed. (1987) Current Protocols in Molecular Biology, Greene Publishing and Wiley Interscience, New York. A nucleic acid described herein can be, for example, DNA or RNA and can or cannot contain intronic sequences. In a certain aspects, the nucleic acid is a cDNA molecule. Nucleic acids described herein can be obtained using standard molecular biology techniques known in the art.

Certain nucleic acid molecules disclosed herein are those encoding an IL-7 protein (e.g., disclosed herein). Exemplary nucleic acid sequences encoding an IL-7 protein disclosed herein are set forth in SEQ ID NOs: 29-39.

In some aspects, the present disclosure provides a vector comprising an isolated nucleic acid molecule encoding a therapeutic agent disclosed herein (e.g., an IL-7 protein). In some aspects, a vector can be used for gene therapy.

When used as a gene therapy (e.g., in humans), a nucleic acid encoding a therapeutic agent disclosed herein (e.g., an IL-7 protein) can be administered at a dosage in the range of 0.1 mg to 200 mg. In certain aspects, the dosage is in the range of 0.6 mg to 100 mg. In further aspects, the dosage is in the range of 1.2 mg to 50 mg.

Suitable vectors for the disclosure include expression vectors, viral vectors, and plasmid vectors. In some aspects, the vector is a viral vector.

As used herein, an expression vector refers to any nucleic acid construct which contains the necessary elements for the transcription and translation of an inserted coding sequence, or in the case of an RNA viral vector, the necessary elements for replication and translation, when introduced into an appropriate host cell. Expression vectors can include plasmids, phagemids, viruses, and derivatives thereof.

As used herein, viral vectors include, but are not limited to, nucleic acid sequences from the following viruses: retrovirus, such as Moloney murine leukemia virus, Harvey murine sarcoma virus, murine mammary tumor virus, and Rous sarcoma virus; lentivirus; adenovirus; adeno-associated virus; SV40-type viruses; polyomaviruses; Epstein-Barr viruses; papilloma viruses; herpes virus; vaccinia virus; polio virus; and RNA virus such as a retrovirus. One can readily employ other vectors well-known in the art. Certain viral vectors are based on non-cytopathic eukaryotic viruses in which non-essential genes have been replaced with the gene of interest. Non-cytopathic viruses include retroviruses, the life cycle of which involves reverse transcription of genomic viral RNA into DNA with subsequent proviral integration into host cellular DNA.

In some aspects, a vector is derived from an adeno-associated virus. In other aspects, a vector is derived from a lentivirus. Examples of the lentiviral vectors are disclosed in WO9931251, W09712622, W09817815, W09817816, and WO9818934, each which is incorporated herein by reference in its entirety.

Other vectors include plasmid vectors. Plasmid vectors have been extensively described in the art and are well-known to those of skill in the art. See, e.g., Sambrook et al., Molecular Cloning: A Laboratory Manual, Second Edition, Cold Spring Harbor Laboratory Press, 1989. In the last few years, plasmid vectors have been found to be particularly advantageous for delivering genes to cells in vivo because of their inability to replicate within and integrate into a host genome. These plasmids, however, having a promoter compatible with the host cell, can express a peptide from a gene operably encoded within the plasmid. Some commonly used plasmids available from commercial suppliers include pBR322, pUC18, pUC19, various pcDNA plasmids, pRC/CMV, various pCMV plasmids, pSV40, and pBlueScript. Additional examples of specific plasmids include pcDNA3.1, catalog number V79020; pcDNA3.1/hygro, catalog number V87020; pcDNA4/myc-His, catalog number V86320; and pBudCE4.1, catalog number V53220, all from Invitrogen (Carlsbad, Calif.). Other plasmids are well-known to those of ordinary skill in the art. Additionally, plasmids can be custom designed using standard molecular biology techniques to remove and/or add specific fragments of DNA.

Also encompassed by the present disclosure is a method for making a therapeutic agent disclosed herein (e.g., an IL-7 protein). In some aspects, such a method can comprise expressing the therapeutic agent (e.g., an IL-7 protein) in a cell comprising a nucleic acid molecule encoding the therapeutic agent, e.g., SEQ ID NOs: 29-39. Additional details regarding the method for making an IL-7 protein disclosed herein are provided, e.g., in WO 2016/200219, which is herein incorporated by reference in its entirety. Host cells comprising these nucleotide sequences are encompassed herein. Non-limiting examples of host cell that can be used include immortal hybridoma cell, NS/0 myeloma cell, 293 cell, Chinese hamster ovary (CHO) cell, HeLa cell, human amniotic fluid-derived cell (CapT cell), COS cell, or combinations thereof.

### V. Pharmaceutical Compositions

Further provided herein are compositions comprising one or more therapeutic agents (e.g., an IL-7 protein and/or a bispecific antibody) having the desired degree of purity in a physiologically acceptable carrier, excipient or stabilizer (Remington's Pharmaceutical Sciences (1990) Mack Publishing Co., Easton, Pa.). In some aspects, a composition disclosed herein comprises an IL-7 protein or a bispecific antibody. As disclosed herein, such compositions can be used in combination (e.g., a first composition comprising an IL-7 protein and a second composition comprising a bispecific antibody). In other aspects, a composition disclosed herein can comprise both an IL-7 protein and a bispecific antibody.

Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN®, PLURONICS® or polyethylene glycol (PEG).

In some aspects, a composition disclosed herein (e.g., comprising an IL-7 protein or an immune checkpoint inhibitor) comprises one or more additional components selected from: a bulking agent, stabilizing agent, surfactant, buffering agent, or combinations thereof.

Buffering agents useful for the current disclosure can be a weak acid or base used to maintain the acidity (pH) of a solution near a chosen value after the addition of another acid or base. Suitable buffering agents can maximize the stability of the pharmaceutical compositions by maintaining pH control of the composition. Suitable buffering agents can also ensure physiological compatibility or optimize solubility. Rheology, viscosity and other properties can also dependent on the pH of the composition. Common buffering agents include, but are not limited to, a Tris buffer, a Tris-Cl buffer, a histidine buffer, a TAE buffer, a HEPES buffer, a TBE buffer, a sodium phosphate buffer, a MES buffer, an ammonium sulfate buffer, a potassium phosphate buffer, a potassium thiocyanate buffer, a succinate buffer, a tartrate buffer, a DIPSO buffer, a HEPPSO buffer, a POPSO buffer, a PIPES buffer, a PBS buffer, a MOPS buffer, an acetate buffer, a phosphate buffer, a cacodylate buffer, a glycine buffer, a sulfate buffer, an imidazole buffer, a guanidine hydrochloride buffer, a phosphate-citrate buffer, a borate buffer, a malonate buffer, a 3-picoline buffer, a 2-picoline buffer, a 4-picoline buffer, a 3,5-lutidine buffer, a 3,4-lutidine buffer, a 2,4-lutidine buffer, a Aces, a diethylmalonate buffer, a N-methylimidazole buffer, a 1,2-dimethylimidazole buffer, a TAPS buffer, a bis-Tris buffer, a L-arginine buffer, a lactate buffer, a glycolate buffer, or combinations thereof.

In some aspects, a composition disclosed herein further comprises a bulking agent. Bulking agents can be added to a pharmaceutical product in order to add volume and mass to the product, thereby facilitating precise metering and handling thereof. Bulking agents that can be used with the present disclosure include, but are not limited to, sodium chloride (NaCl), mannitol, glycine, alanine, or combinations thereof.

In some aspects, a composition disclosed herein can also comprise a stabilizing agent. Non-limiting examples of stabilizing agents that can be used with the present disclosure include: sucrose, trehalose, raffinose, arginine, or combinations thereof.

In some aspects, a composition disclosed herein comprises a surfactant. In certain aspects, the surfactant can be selected from the following: alkyl ethoxylate, nonylphenol ethoxylate, amine ethoxylate, polyethylene oxide, polypropylene oxide, fatty alcohols such as cetyl alcohol or oleyl alcohol, cocamide MEA, cocamide DEA, polysorbates, dodecyl dimethylamine oxide, or combinations thereof. In some aspects, the surfactant is polysorbate 20 or polysorbate 80.

In some aspects, a composition comprising an IL-7 protein can be formulated using the same formulation of a bispecific antibody (e.g., which is to be used in combination with the IL-7 protein). In other aspects, an IL-7 protein and a bispecific antibody are formulated using different formulations.

In some aspects, an IL-7 protein disclosed herein is formulated in a composition comprising (a) a basal buffer, (b) a sugar, and (c) a surfactant. In certain aspects, the basal buffer comprises histidine-acetate or sodium citrate. In some aspects, the basal buffer is at a concentration of about 10 to about 50 nM. In some aspects, a sugar comprises sucrose, trehalose, dextrose, or combinations thereof. In some aspects, the sugar is present at a concentration of about 2.5 to about 5.0 w/v %. In further aspects, the surfactant is selected from polysorbate, polyoxyethylene alkyl ether, polyoxyethylene stearate, alkyl sulfates, polyvinyl pyridone, poloxamer, or combinations thereof. In some embodiments, the surfactant is at a concentration of about 0.05% to about 6.0 w/v %.

In some aspects, the composition in which IL-7 is formulated further comprises an amino acid. In certain embodiments, the amino acid is selected from arginine, glutamate, glycine, histidine, or combinations thereof. In certain aspects, the composition further comprises a sugar alcohol. Non-limiting examples of sugar alcohol includes: sorbitol, xylitol, maltitol, mannitol, or combinations thereof.

In some aspects, an IL-7 protein disclosed herein is formulated in a composition comprising the following: (a) sodium citrate (e.g., about 20 mM), (b) sucrose (e.g., about 5%), (c) sorbitol (e.g., about 1.5%), and (d) Tween 80 (e.g., about 0.05%).

In some aspects, an IL-7 protein of the present disclosure is formulated as described in WO 2017/078385 A1, which is incorporated herein in its entirety.

A pharmaceutical composition can be formulated for any route of administration to a subject. Specific examples of routes of administration include intramuscularly, subcutaneously, ophthalmic, intravenously, intraperitoneally, intradermally, intraorbitally, intracerebrally, intracranially, intraspinally, intraventricular, intrathecally, intracistemally, intracapsularly, or intratumorally. Parenteral administration, characterized by either subcutaneous, intramuscular or intravenous injection, is also contemplated herein. In some aspects, an IL-7 protein and a bispecific antibody are administered using the same route of administration. In other aspects, an IL-7 protein and a bispecific antibody are administered using different routes of administration. In some aspects, a bispecific antibody is administered intravenously or intratumorally. In certain aspects, a bispecific antibody is administered intratumorally.

Injectables can be prepared in conventional forms, either as liquid solutions or suspensions, solid forms suitable for solution or suspension in liquid prior to injection, or as emulsions. The injectables, solutions and emulsions also contain one or more excipients. Suitable excipients are, for example, water, saline, dextrose, glycerol or ethanol. In addition, if desired, the pharmaceutical compositions to be administered can also contain minor amounts of non-toxic auxiliary substances such as wetting or emulsifying agents, pH buffering agents, stabilizers, solubility enhancers, and other such agents, such as for example, sodium acetate, sorbitan monolaurate, triethanolamine oleate and cyclodextrins.

Pharmaceutically acceptable carriers used in parenteral preparations include aqueous vehicles, nonaqueous vehicles, antimicrobial agents, isotonic agents, buffers, antioxidants, local anesthetics, suspending and dispersing agents, emulsifying agents, sequestering or chelating agents and other pharmaceutically acceptable substances. Examples of aqueous vehicles include Sodium Chloride Injection, Ringers Injection, Isotonic Dextrose Injection, Sterile Water Injection, Dextrose and Lactated Ringers Injection. Nonaqueous parenteral vehicles include fixed oils of vegetable origin, cottonseed oil, corn oil, sesame oil and peanut oil. Antimicrobial agents in bacteriostatic or fungistatic concentrations can be added to parenteral preparations packaged in multiple-dose containers which include phenols or cresols, mercurials, benzyl alcohol, chlorobutanol, methyl and propyl p-hydroxybenzoic acid esters, thimerosal, benzalkonium chloride and benzethonium chloride. Isotonic agents include sodium chloride and dextrose. Buffers include phosphate and citrate. Antioxidants include sodium bisulfate. Local anesthetics include procaine hydrochloride. Suspending and dispersing agents include sodium carboxymethylcelluose, hydroxypropyl methylcellulose and polyvinylpyrrolidone. Emulsifying agents include Polysorbate 80 (TWEEN® 80). A sequestering or chelating agent of metal ions includes EDTA. Pharmaceutical carriers also include ethyl alcohol, polyethylene glycol and propylene glycol for water miscible vehicles; and sodium hydroxide, hydrochloric acid, citric acid or lactic acid for pH adjustment.

Preparations for parenteral administration include sterile solutions ready for injection, sterile dry soluble products, such as lyophilized powders, ready to be combined with a solvent just prior to use, including hypodermic tablets, sterile suspensions ready for injection, sterile dry insoluble products ready to be combined with a vehicle just prior to use and sterile emulsions. The solutions can be either aqueous or nonaqueous.

If administered intravenously, suitable carriers include physiological saline or phosphate buffered saline (PBS), and solutions containing thickening and solubilizing agents, such as glucose, polyethylene glycol, and polypropylene glycol and mixtures thereof.

Topical mixtures comprising an antibody are prepared as described for the local and systemic administration. The resulting mixture can be a solution, suspension, emulsions or the like and can be formulated as creams, gels, ointments, emulsions, solutions, elixirs, lotions, suspensions, tinctures, pastes, foams, aerosols, irrigations, sprays, suppositories, bandages, dermal patches or any other formulations suitable for topical administration.

A therapeutic agent described herein (e.g., an IL-7 protein or bispecific antibody) can be formulated as an aerosol for topical application, such as by inhalation (see, e.g., U.S. Pat. Nos. 4,044,126, 4,414,209 and 4,364,923, which describe aerosols for delivery of a steroid useful for treatment of inflammatory diseases, particularly asthma). These formulations for administration to the respiratory tract can be in the form of an aerosol or solution for a nebulizer, or as a microfine powder for insufflations, alone or in combination with an inert carrier such as lactose. In such a case, the particles of the formulation will, in one aspect, have diameters of less than 50 microns, in one aspect less than 10 microns.

A therapeutic agent disclosed herein (e.g., an IL-7 protein or bispecific antibody) can be formulated for local or topical application, such as for topical application to the skin and mucous membranes, such as in the eye, in the form of gels, creams, and lotions and for application to the eye or for intracisternal or intraspinal application. Topical administration is contemplated for transdermal delivery and also for administration to the eyes or mucosa, or for inhalation therapies. Nasal solutions of the antibody alone or in combination with other pharmaceutically acceptable excipients can also be administered.

Transdermal patches, including iontophoretic and electrophoretic devices, are well known to those of skill in the art, and can be used to administer an antibody. For example, such patches are disclosed in U.S. Pat. Nos. 6,267,983, 6,261,595, 6,256,533, 6,167,301, 6,024,975, 6,010715, 5,985,317, 5,983,134, 5,948,433, and 5,860,957, each of which is herein incorporated by reference in its entirety.

In certain aspects, a pharmaceutical composition comprising a therapeutic agent described herein (e.g., an IL-7 protein or bispecific antibody) is a lyophilized powder, which can be reconstituted for administration as solutions, emulsions and other mixtures. It can also be reconstituted and formulated as solids or gels. The lyophilized powder is prepared by dissolving an antibody or antigen-binding portion thereof described herein, or a pharmaceutically acceptable derivative thereof, in a suitable solvent. In some aspects, the lyophilized powder is sterile. The solvent can contain an excipient which improves the stability or other pharmacological component of the powder or reconstituted solution, prepared from the powder. Excipients that can be used include, but are not limited to, dextrose, sorbitol, fructose, corn syrup, xylitol, glycerin, glucose, sucrose or other suitable agent. The solvent can also contain a buffer, such as citrate, sodium or potassium phosphate or other such buffer known to those of skill in the art at, in one aspect, about neutral pH. Subsequent sterile filtration of the solution followed by lyophilization under standard conditions known to those of skill in the art provides the desired formulation. In some aspects, the resulting solution can be apportioned into vials for lyophilization. Each vial can contain a single dosage or multiple dosages of the compound. The lyophilized powder can be stored under appropriate conditions, such as at about 4° C. to room temperature.

Reconstitution of this lyophilized powder with water for injection provides a formulation for use in parenteral administration. For reconstitution, the lyophilized powder is added to sterile water or other suitable carrier. The precise amount depends upon the selected compound. Such amount can be empirically determined.

Compositions provided herein can also be formulated to be targeted to a particular tissue, receptor, or other area of the body of the subject to be treated. Many such targeting methods are well known to those of skill in the art. All such targeting methods are contemplated herein for use in the instant compositions. For non-limiting examples of targeting methods, see, e.g., U.S. Pat. Nos. 6,316,652, 6,274,552, 6,271,359, 6,253,872, 6,139,865, 6,131,570, 6,120,751, 6,071,495, 6,060,082, 6,048,736, 6,039,975, 6,004,534, 5,985,307, 5,972,366, 5,900,252, 5,840,674, 5,759,542 and 5,709,874. In a specific aspect, the anti-FAM19A5 antibody or antigen-binding portion thereof described herein can be used treat a fibrosis and/or a disease or condition associated with fibrosis.

The compositions to be used for in vivo administration can be sterile. This is readily accomplished by filtration through, e.g., sterile filtration membranes.

The following examples are merely illustrative and should not be construed as limiting the scope of this disclosure in any way as many variations and equivalents will become apparent to those skilled in the art upon reading the present disclosure.

### EXAMPLES

### Example 1: Analysis of the Anti-Tumor Effects of Anti-PD-L1/CD3 Bi-Specific T-Cell Engager (BsAb)

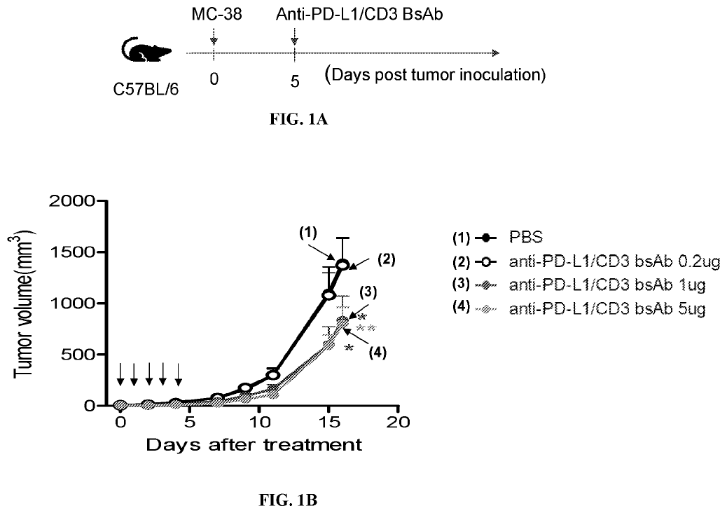

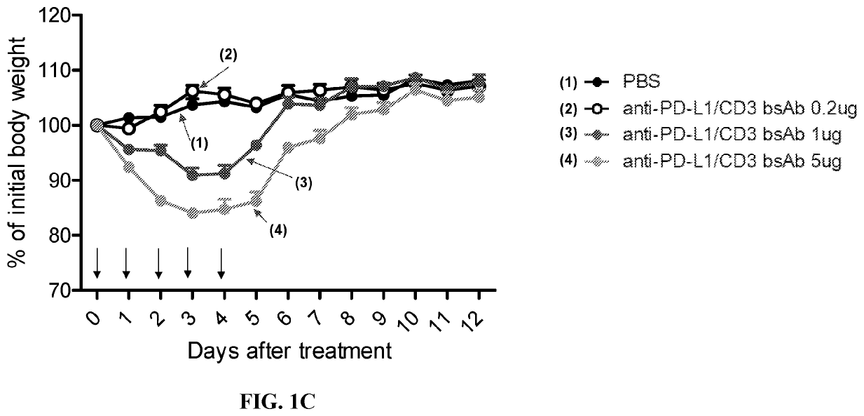

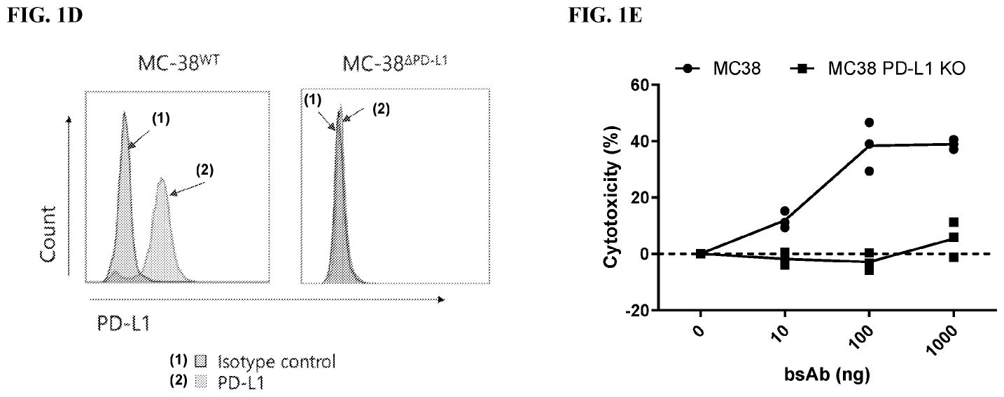

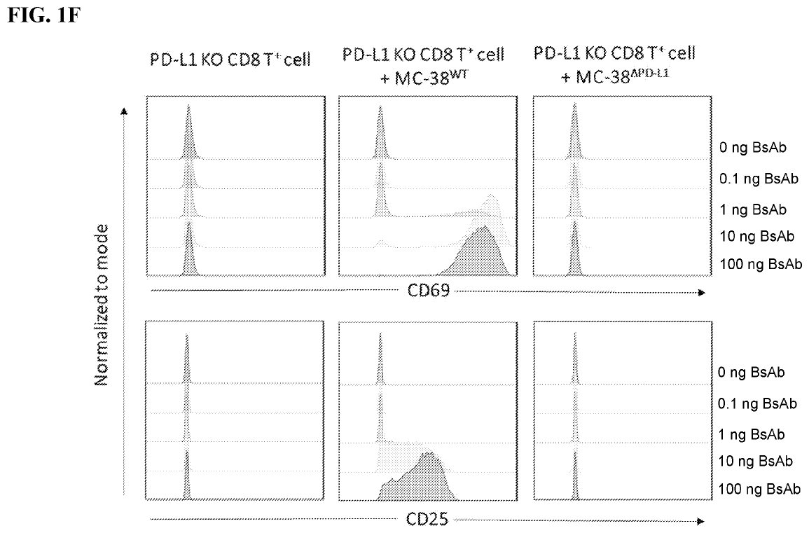

To assess the anti-tumor effects of an anti-PD-L1/CD3 bi-specific T-cell engager (“BsAb”) antibody (SEQ ID NO: 73), a colon adenocarcinoma mouse model was used. Briefly, MC-38 colon adenocarcinoma tumor cells (1×10⁵, subcutaneously) were transplanted into C57BL/6 mice. Palpable tumors were detected on day 5 post tumor inoculation. On day 5 post tumor inoculation, the animals were intravenously administered phosphate buffered saline (“PBS”) (control group), or 0.2 μg BsAb, 1.0 μg BsAb, or 5.0 μg BsAb daily for 5 days. See FIG. 1A. Both tumor volume and body weight were measured periodically after the last treatment. Body weight was measured as a marker for toxicity of the antibody in vivo. FIG. lA provides a graphical depiction of the dosing schedule and Table 1 (below) provides the different treatment groups.

TABLE 1
Treatment Groups

| Group | Administered Dose | Number of Animals |
|---|---|---|
| Phosphate buffered | | 5 |
| saline (Control) | | |
| Anti-PD-L1/CD3 BsAb | 0.2 μg per day for 5 days | 5 |
| Anti-PD-L1/CD3 BsAb | 1.0 μg per day for 5 days | 5 |
| Anti-PD-L1/CD3 BsAb | 5.0 μg per day for 5 days | 4 |

As shown in FIG. 1B, the tumor volumes of the control group and the animals treated with 0.2 μg BsAb per day for 5 days significantly increased, as compared to the animals in the 1.0 μg BsAb and 5.0 μg BsAb treatment groups. The tumor volumes of animal in the control group and the 0.2 μg BsAb treatment group increased to about 1300 mm³. In contrast, the tumor volumes of the animals in the 1.0 μg BsAb and 5.0 μg BsAb treatment groups were significantly lower, suggesting that BsAb at a dose of 0.2 μg was ineffective in treating tumor. Between 1.0 μg BsAb and 5.0 μg BsAb, there did not appear to be a significant difference on tumor volume.

Consistent with the lack of anti-tumor efficacy, 0.2 μg BsAb treatment group exhibited minimal body weight loss after the treatment. See FIG. 1C. In both the 1.0 μg BsAb and 5.0 μg BsAb treatment groups, there was a significant decrease in body weight soon after the treatment, but the animals eventually returned to normal body weight. However, the loss of body weight was much greater and the animals took longer to return to normal body weight in the 5.0 μg BsAb treatment group. This result suggested that BsAb at too high of a dose may be toxic with minimal increase in therapeutic effects.

Next, to confirm that the anti-PD-L1/CD3 BsAb is capable of targeting PD-L1 expressed on tumor cells, splenocytes (comprising CD8+ T cells) were isolated from PD-L1 knockout mice and co-cultured for 48 hours with either MC-38 wild-type (“MC-38^{WT}”; expressed PD-L1, see FIG. 1D) or PD-L1 -depleted (“MC-38^{ΔPD-L1}”) tumor cells in the presence of varying concentrations of the BsAb (i.e., 0 ng, 0.1 ng, 1 ng, 10 ng, 100 ng, or 1000 ng). Then, both the cytotoxicity and the activation of the CD8+ T cells were assessed.

As shown in FIG. 1E, in the presence of BsAb (e.g., starting at about 1-10 ng), the CD8+ T cells were able to specifically target wild-type MC-38 tumor cells. In support, the CD8+ T cells that were cultured with the wild-type MC-38 tumor cells in the presence of BsAb also had an activated phenotype (as evidenced by increased CD69 and CD25 expression) (see FIG. 1F).

Collectively, the results provided in this Example demonstrate that the anti-tumor effects of the anti-PD-L1/CD3 BsAbs described herein.

### Example 2: Analysis of the Anti-Tumor Effect of Anti-PD-L1/CD3 BsAb and IL-7 Protein Combination Therapy

To assess whether an IL-7 protein disclosed herein can improve the anti-tumor effects of anti-PD-L1/CD3 BsAb, MC-38 colon adenocarcinoma tumor cells (1×10⁵, subcutaneously) were again transplanted into C57BL/6 mice. Palpable tumors were detected on day 7 post tumor inoculation. On day 7 post tumor inoculation, the animals subcutaneously received a single dose (1.25 mpk) of a modified IL-7 protein (e.g., disclosed herein) or buffer alone. Three days later (day 10 post tumor inoculation), the animals were treated intravenously with different doses of BsAb or PBS daily for five days. The different treatment groups are shown in Table 2 (below). After the last treatment, the animals were monitored over a course of about thirteen days, and both tumor volume and the body weight of the animals were measured.

TABLE 2
Combination Therapy Treatment Groups

| | | | Number |
|---|---|---|---|
| | | | of |
| Group | Treatment | Administered Dose | Animals |
| Control | Buffer + PBS | | 6 |
| 1 | IL-7 + PBS | Single 1.25 mpk dose of IL-7 | 5 |
| | | and PBS daily for 5 days | |
| 2 | Buffer + anti- | 1.0 μg BsAb per day for 5 days | 6 |
| | PD-L1/CD3 | | |
| | BsAb | | |
| 3 | IL-7 + anti-PD- | Single dose of IL-7 and | 7 |
| | L1/CD3 BsAb | 0.04 μg BsAb per day for 5 days | |
| 4 | IL-7 + anti-PD- | Single dose of IL-7 and | 6 |
| | L1/CD3 BsAb | 0.2 μg BsAb per day for 5 days | |
| 5 | IL-7 + anti-PD- | Single dose of IL-7 and | 4 |
| | L1/CD3 BsAb | 1.0 μg BsAb per day for 5 days | |

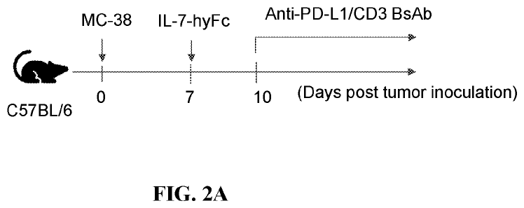

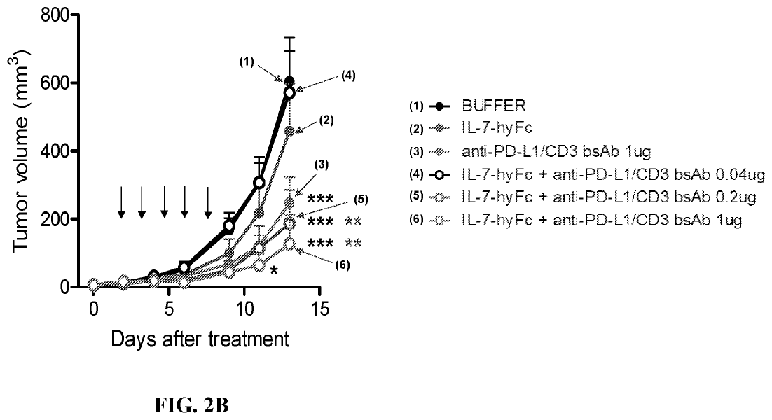

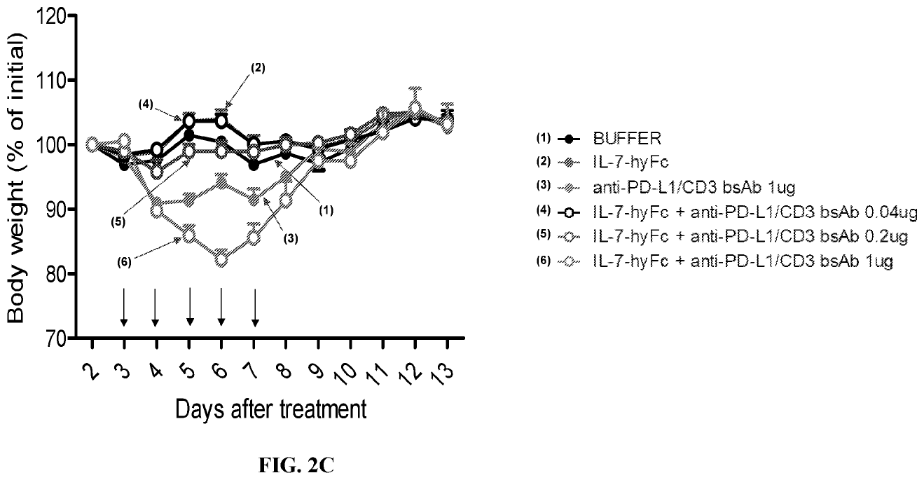

Consistent with the earlier Example, animals treated with 1 μg BsAb alone had decreased tumor volume and moderate loss in body weight that eventually returned to normal. See FIGS. 2B and 2C. Surprisingly, animals treated with 0.2 μg BsAb (which was shown to have no anti-tumor effects when administered alone) in combination with the modified IL-7 protein exhibited significant anti-tumor effects (FIG. 2B) with minimal loss in body weight (FIG. 2C). Compared to the 1 μg BsAb alone treated group, animals treated with the combination of 0.2 μg BsAb and IL-7 protein not only had moderately improved anti-tumor effects but more importantly, had significantly less body weight loss. The most favorable anti-tumor effects with minimal toxicity was observed in animals treated with 0.2 μg BsAb and IL-7 protein.

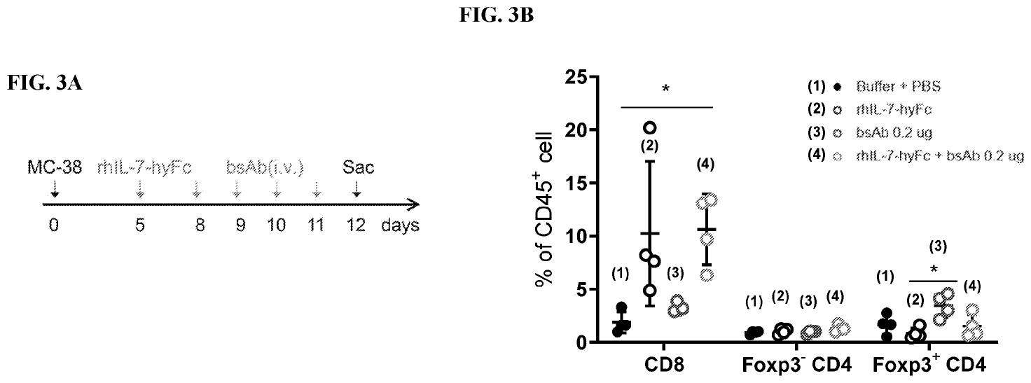

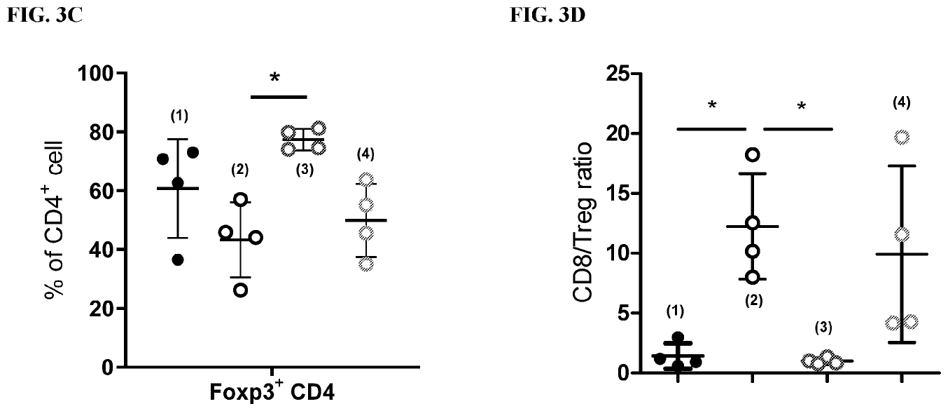

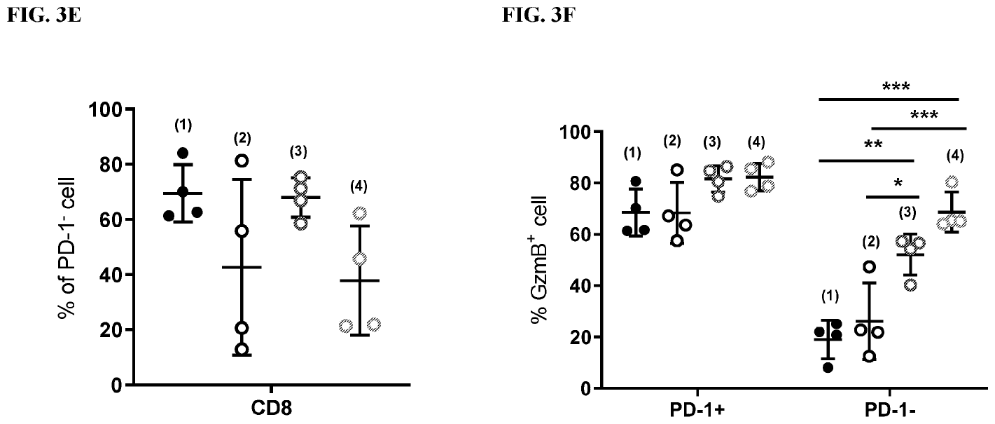

To further assess the above effects, MC-38 tumor animals were treated with one of the following as shown in FIG. 3A: (i) no treatment; (ii) IL-7 protein alone (subcutaneously); (iii) BsAb alone (0.2 μg; intravenously); and (iv) IL-7 protein (subcutaneously) in combination with BsAb (0.2 μg; intravenously). A single dose of the IL-7 protein (1.25 mpk) was administered to the relevant animals at day 5 post tumor inoculation. The BsAb was administered to the relevant animals on days 8, 9, 10, and 11 post tumor inoculation for a total of four administrations (each administration=0.2 μg). Then, the animals were sacrificed at day 12 post-tumor inoculation, and both the frequency and phenotypic characteristics of the different T cell populations were assessed.

As shown in FIGS. 3B-3F, animals treated with the combination of IL-7 and BsAb exhibited increased frequency of CD8+ T cells (including bystander CD8+ T cells) in the tumors compared to animals that received no treatment or treated with BsAb alone. Animals from the combination group also appeared to have reduced number of Foxp3+ regulatory CD4+ T cells (see FIGS. 3B-3D). In addition to the increased frequency, the CD8+ T cells in the tumors of animals (as well as the bystander CD8+ T cells) treated with both the IL-7 and BsAb were also largely PD-1− and had greater expression of granzyme B (see FIGS. 3E and 3F), demonstrating their improved effector phenotype. Moreover, the activation of the bystander CD8+ T cells suggests that the observed antitumor responses could be partially mediated by the targeted activation of bystander CD8+ T cells.

The above results demonstrate the IL-7 proteins disclosed herein can greatly improve the anti-tumor effects of systemically administered bispecific antibodies, such as BsAb, while causing minimal toxicity.

### Example 3: Anti-Tumor Effect of Anti-PD-L1/CD3 BsAb and IL-7 Protein Combination Therapy After Intratumoral Administration

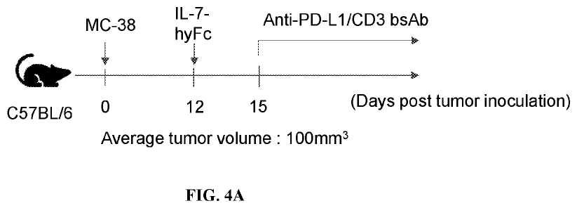

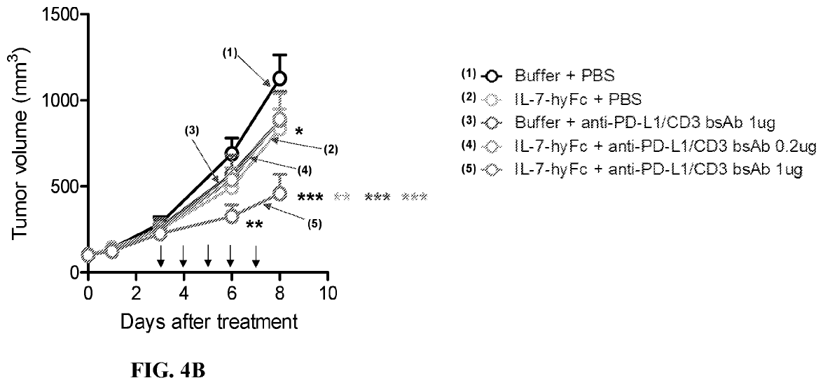

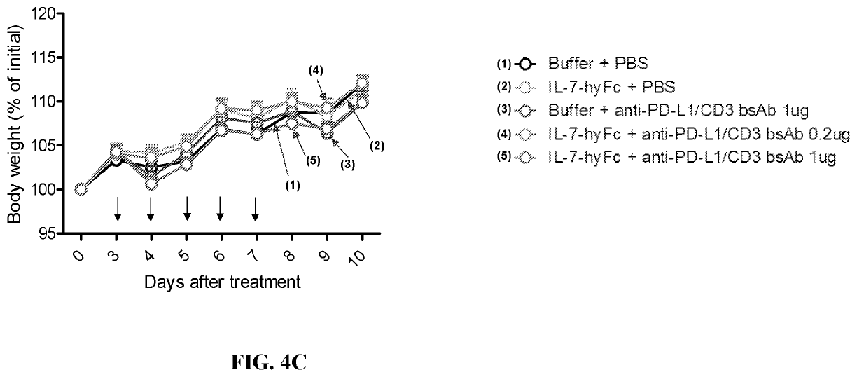

To assess whether administration route can have an effect on the anti-tumor effects of PD-L1/CD3 BsAb and IL-7 protein combination treatment, a colon adenocarcinoma mouse model were used again. Briefly, MC-38 colon adenocarcinoma tumor cells were transplanted into mice as described above in Examples 1 and 2. Once palpable tumors were confirmed (tumor diameter of between 5 mm-7 mm and/or tumor volume of about 100±30 mm³) (i.e., around day 12 post tumor inoculation), the animals received a single subcutaneous administration of buffer alone or a modified IL-7 protein (e.g., disclosed herein). Three days later, the animals received different doses of anti-PD-L1/CD3 BsAb intratumorally daily for five days. After the last administration, both tumor volume and body weight of the animals were measured. FIG. 4A provides a graphical depiction of the dosing schedule and Table 3 (below) provides the different treatment groups.

TABLE 3
Combination Therapy Treatment Groups

| | | | Number |
|---|---|---|---|
| | | | of |
| Group | Treatment | Dose | Animals |
| Control | PBS | | 9 |
| 1 | IL-7 + PBS | Single 1.25 mpk dose of IL-7 | 9 |
| | | and PBS daily for 5 days | |
| 2 | Buffer + anti- | Buffer + 1.0 μg BsAb per day | 10 |
| | PD-L1/CD3 | for 5 days | |
| | BsAb | | |
| 3 | IL-7 + anti-PD- | Single 1.25 mpk dose of IL-7, | 10 |
| | L1/CD3 BsAb | and 0.2 μg BsAb per day for 5 days | |
| 4 | IL-7 + anti-PD- | Single 1.25 mpk dose of IL-7, | 10 |
| | L1/CD3 BsAb | and 1.0 μg BsAb per day for 5 days | |

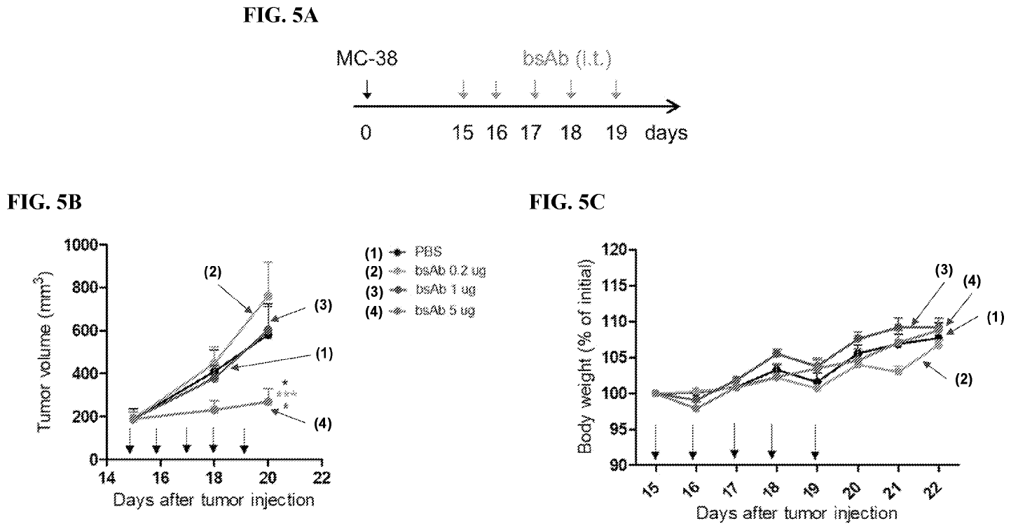

Contrary to the results observed in Examples 1 and 2 (where BsAb was administered to the animals intravenously), animals that received an intratumoral administration of 1 μg BsAb alone did not exhibit decreased tumor volume compared to the control animals (e.g., animals that received IL-7 protein alone, see FIG. 4B). With BsAb intratumoral administration alone, much higher doses of the BsAb (e.g., 5 μg) were required to observe an anti-tumor effect (see FIGS. 5A-5C). However, when administered in combination with the IL-7 protein, there was a significant decrease in tumor volume in the animals treated with the 1 μg BsAb, demonstrating the synergistic effect of the combination treatment. As for body weight, no noticeable loss was observed in any of the treatment groups, suggesting that intratumoral administration of the BsAb had no systemic toxicity (see FIG. 4C). The above results demonstrate that the IL-7 proteins disclosed herein can greatly improve the anti-tumor effects of bispecific antibodies, such as BsAb, with no toxicity through intratumoral administration.

Collectively, the results described above suggest that the combination treatment described herein (IL-7 protein in combination with bispecific antibodies) could be a promising strategy for cancer immunotherapy.

What is claimed is:

1. A method of treating a tumor in a subject in need thereof, comprising administering to the subject an effective amount of a modified IL-7 protein and a bispecific antibody.

2. The method of claim 1, wherein a tumor volume is reduced in the subject after the administration compared to a reference tumor volume (e.g., tumor volume in the subject prior to administration and/or tumor volume in a subject after administration of either the modified IL-7 protein or the bispecific antibody alone).

3. The method of claim 2, wherein the tumor volume is reduced by at least about 5%, at least about 10%, at least about 20%, at least about 30%, at least about 40%, at least about 50%, at least about 60%, at least about 70%, at least about 80%, at least about 90%, or at least about 100% after the administration compared to the reference tumor volume.

4. The method of any one of claims 1 to 3, wherein a body weight of the subject is not decreased after the administration compared to a reference body weight (e.g., body weight of the subject prior to administration and/or body weight of a subject after administration of either the modified IL-7 protein or the bispecific antibody alone).

5. The method of claim 4, wherein the body weight of the subject is not decreased by more than about 1%, more than about 2%, more than about 3%, more than about 4%, more than about 5%, more than about 6%, more than about 7%, more than about 8%, more than about 9%, or more than about 10% after the administration compared to the reference body weight.

6. A method of enhancing an anti-tumor activity of a bispecific antibody in a subject in need thereof, comprising administering to the subject an effective amount of a bispecific antibody in combination with a modified IL-7 protein.

7. The method of claim 6, wherein the anti-tumor activity comprises a reduction in tumor volume and/or lack of loss of body weight in the subject.

8. The method of claim 7, wherein the tumor volume is reduced by at least about 5%, at least about 10%, at least about 20%, at least about 30%, at least about 40%, at least about 50%, at least about 60%, at least about 70%, at least about 80%, at least about 90%, or at least about 100% after the administration compared to a reference tumor volume (e.g., tumor volume in the subject prior to administration and/or tumor volume in a subject after administration of either the modified IL-7 protein or the bispecific antibody alone).

9. The method of claim 7 or 8, wherein the body weight of the subject is not decreased by more than about 1%, more than about 2%, more than about 3%, more than about 4%, more than about 5%, more than about 6%, more than about 7%, more than about 8%, more than about 9%, or more than about 10% after the administration compared to a reference body weight (e.g., body weight of the subject prior to administration and/or body weight of a subject after administration of either the modified IL-7 protein or the bispecific antibody alone).

10. The method of any one of claims 1 to 9, wherein the modified IL-7 protein comprises an oligopeptide consisting of 1 to 10 amino acid residues.

11. The method of claim 10, wherein the oligopeptide comprises methionine (M), glycine (G), methionine-methionine (MM), glycine-glycine (GG), methionine-glycine (MG), glycine-methionine (GM), methionine-methionine-methionine (MMM), methionine-methionine-glycine (MMG), methionine-glycine-methionine (MGM), glycine-methionine-methionine (GMM), methionine-glycine-glycine (MGG), glycine-methionine-glycine (GMG), glycine-glycine-methionine (GGM), glycine-glycine-glycine (GGG), methionine-glycine-glycine-methionine (MGGM) (SEQ ID NO: 41), methionine-methionine-glycine-glycine (MMGG) (SEQ ID NO: 42), glycine-glycine-methionine-methionine (GGMM) (SEQ ID NO: 43), methionine-glycine-methionine-glycine (MGMG) (SEQ ID NO: 44), glycine-methionine-methionine-glycine (GMMG) (SEQ ID NO: 45), glycine-glycine-glycine-methionine (GGGM) (SEQ ID NO: 46), methionine-glycine-glycine-glycine (MGGG) (SEQ ID NO: 47), glycine-methionine-glycine-glycine (GMGG) (SEQ ID NO: 48), glycine-glycine-methionine-glycine (GGMG) (SEQ ID NO: 49), glycine-glycine-methionine-methionine-methionine (GGMMM) (SEQ ID NO: 50), glycine-glycine-glycine-methionine-methionine (GGGMM) (SEQ ID NO: 51), glycine-glycine-glycine-glycine-methionine (GGGGM) (SEQ ID NO: 52), methionine-glycine-methionine-methionine-methionine (MGMMM) (SEQ ID NO: 53), methionine-glycine-glycine-methionine-methionine (MGGMM) (SEQ ID NO: 54), methionine-glycine-glycine-glycine-methionine (MGGGM) (SEQ ID NO: 55), methionine-methionine-glycine-methionine-methionine (MMGMM) (SEQ ID NO: 56), methionine-methionine-glycine-glycine-methionine (MMGGM) (SEQ ID NO: 57), methionine-methionine-glycine-glycine-glycine (MMGGG) (SEQ ID NO: 58), methionine-methionine-methionine-glycine-methionine (MMMGM) (SEQ ID NO: 59), methionine-glycine-methionine-glycine-methionine (MGMGM) (SEQ ID NO: 60), glycine-methionine-glycine-methionine-glycine (GMGMG) (SEQ ID NO: 61), glycine-methionine-methionine-methionine-glycine (GMMMG) (SEQ ID NO: 62), glycine-glycine-methionine-glycine-methionine (GGMGM) (SEQ ID NO: 63), glycine-glycine-methionine-methionine-glycine (GGMMG) (SEQ ID NO: 64), glycine-methionine-methionine-glycine-methionine (GMMGM) (SEQ ID NO: 65), methionine-glycine-methionine-methionine-glycine (MGMMG) (SEQ ID NO: 66), glycine-methionine-glycine-glycine-methionine (GMGGM) (SEQ ID NO: 67), methionine-methionine-glycine-methionine-glycine (MMGMG) (SEQ ID NO: 68), glycine-methionine-methionine-glycine-glycine (GMMGG) (SEQ ID NO: 69), glycine-methionine-glycine-glycine-glycine (GMGGG) (SEQ ID NO: 70), glycine-glycine-methionine-glycine-glycine (GGMGG) (SEQ ID NO: 71), glycine-glycine-glycine-glycine-glycine (GGGGG) (SEQ ID NO: 72), or combinations thereof.

12. The method of claim 11, wherein the oligopeptide is methionine-glycine-methionine (MGM).

13. The method of any one of claims 1 to 12, wherein the modified IL-7 protein comprises a half-life extending moiety.

14. The method of claim 13, wherein the half-life extending moiety comprises an Fc, albumin, an albumin-binding polypeptide, Pro/Ala/Ser (PAS), a C-terminal peptide (CTP) of the β subunit of human chorionic gonadotropin, polyethylene glycol (PEG), long unstructured hydrophilic sequences of amino acids (XTEN), hydroxyethyl starch (HES), an albumin-binding small molecule, or a combination thereof.

15. The method of claim 14, wherein the half-life extending moiety is an Fc.

16. The method of claim 15, wherein the Fc is a hybrid Fc, comprising a hinge region, a CH2 domain, and a CH3 domain,
wherein the hinge region comprises a human IgD hinge region,
wherein the CH2 domain comprises a part of human IgD CH2 domain and a part of human IgG4 CH2 domain, and
wherein the CH3 domain comprises a part of human IgG4 CH3 domain.

17. The method of any one of claims 1 to 16, wherein the modified IL-7 protein comprises an amino acid sequence having a sequence identity of at least about 70%, at least about 75%, at least about 80%, at least about 85%, at least about 90%, at least about 95%, at least about 96%, at least about 97%, at least about 98%, at least about 99%, or about 100% to SEQ ID NOs: 1-6 and 15-25.

18. The method of any one of claims 1 to 17, wherein the bispecific antibody comprises a T cell engager (e.g., bispecific T-cell engager (BiTE) antibody), dual-affinity retargeting molecule (DART), CrossMAb antibody, DutaMab™ antibody, DuoBody antibody, Triomab, TandAb, bispecific NanoBody, Tandem scFv, diabody, single chain diabody, HSA body, (scFv)2 HSA Antibody, scFv-IgG antibody, Dock and Lock bispecific antibody, DVD-IgG antibody, TBTI DVD-IgG, IgG-fynomer, Tetravalent bispecific tandem IgG antibody, dual-targeting domain antibody, chemically linked bispecific (Fab′)2 molecule, crosslinked mAb, Dual-action Fab IgG (DAF-IgG), orthoFab-IgG, bispecific CovX-Body, bispecific hexavalent trimerbody, 2 scFv linked to diphtheria toxin, ART-Ig, IgM T-cell engager, or combinations thereof.

19. The method of claim 18, wherein the bispecific antibody comprises a T-cell engager (e.g., bispecific T cell engager (BiTE) antibody).

20. The method of any one of claims 1 to 19, wherein the bispecific antibody binds to a tumor antigen and an antigen expressed on an immune cell.

21. The method of claim 20, wherein the antigen expressed on an immune cell comprises CD2, CD3, CD4, CD5, CD8, CD11b, CD14, CD16, CD19, CD28, CD32, CD45, CD56, CD64, KLRG-1, NKG2D, NKp30, DNAM-1, or combinations thereof.

22. The method of claim 20 or 21, wherein the tumor antigen comprises guanylate cyclase C (GC-C), epidermal growth factor receptor (EGFR or erbB-1), human epidermal growth factor receptor 2 (HER2 or erbB2), erbB-3, erbB-4, MUC-1, melanoma-associated chondroitin sulfate proteoglycan (MCSP), mesothelin (MSLN), folate receptor 1 (FOLR1), CD4, CD19, CD20, CD22, CD30, CD33, CD38, CD44, CD44v6, CD44v7/8, CD70, CD123, CD138, CD171, CEA, CSPG4, CXCR5, c-Met, HERV-envelope protein, eriostin, Bigh3, SPARC, BCR, CD79, CD37, EGFRvIII, EGP2, EGP40, IGFr, L1CAM, AXL, Tissue Factor (TF), CD74, EpCAM, EphA2, MRP3cadherin 19 (CDH19), epidermal growth factor 2 (HER2), 5T4, 8H9, αᵥβ₆ integrin, BCMA, B7-H3, B7-H6, CAIX, CA9, FAP, FBP, fetal AchR, FRcc, GD2, GD3, Glypican-1 (GPC1), Glypican-2 (GPC2), Glypican-3 (GPC3), HLA-A1+MAGE1, HLA-A1+NY-ESO-1, IL-13Rcc2, Lewis-Y, KDR, MCSP, Mesothelin, Muc1, Muc16, NCAM, NKG2D ligands, NY-ESO-1, PRAME, PSC1, PSCA, PSMA, ROR1, ROR2, SP17, surviving, TAG72, TEMs, carcinoembryonic antigen, HMW-MAA, VEGF, CLDN18.2, or combinations thereof.

23. The method of claim 20 or 21, wherein the tumor antigen comprises an immune checkpoint molecule.

24. The method of claim 23, wherein the immune checkpoint molecule comprises a PD-1 ligand (e.g., PD-L1), LAG3 ligand, TIM-3 ligand (e.g., galectin 9), CTLA-4 ligand (e.g., CD28), OX40 ligand, CD28 ligand (e.g., B7H3 or B7H4), or combinations thereof.

25. The method of any one of claims 20 to 24, wherein the immune cell comprises a T-cell.

26. The method of claim 25 wherein the T-cell comprises a tumor-infiltrating lymphocyte (TIL).

27. The method of any one of claims 1 to 26, wherein the modified IL-7 protein and the bispecific antibody are administered concurrently.

28. The method of any one of claims 1 to 26, wherein the modified IL-7 protein and the bispecific antibody are administered sequentially.

29. The method of claim 28, wherein the IL-7 protein is administered to the subject prior to administering the bispecific antibody.

30. The method of any one of claims 1 to 29, wherein the modified IL-7 protein is administered at a dose of greater than about 600 μg/kg, greater than about 700 μg/kg, greater than about 800 μg/kg, greater than about 900 μg/kg, greater than about 1,000 μg/kg, greater than about 1,100 μg/kg, greater than about 1,200 μg/kg, greater than about 1,300 μg/kg, greater than about 1,400 μg/kg, greater than about 1,500 μg/kg, greater than about 1,600 μg/kg, greater than about 1,700 μg/kg, greater than about 1,800 μg/kg, greater than about 1,900 μg/kg, or greater than about 2,000 μg/kg.

31. The method of any one of claims 1 to 30, wherein the modified IL-7 protein is administered at a dose of between about 610 μg/kg and about 1,200 μg/kg, between about 650 μg/kg and about 1,200 μg/kg, between about 700 μg/kg and about 1,200 μg/kg, between about 750 μg/kg and about 1,200 μg/kg, between about 800 μg/kg and about 1,200 μg/kg, between about 850 μg/kg and about 1,200 μg/kg, between about 900 μg/kg and about 1,200 μg/kg, between about 950 μg/kg and about 1,200 μg/kg, between about 1,000 μg/kg and about 1,200 μg/kg, between about 1,050 μg/kg and about 1,200 μg/kg, between about 1,100 μg/kg and about 1,200 μg/kg, between about 1,200 μg/kg and about 2,000 μg/kg, between about 1,300 μg/kg and about 2,000 μg/kg, between about 1,500 μg/kg and about 2,000 μg/kg, between about 1,700 μg/kg and about 2,000 μg/kg, between about 610 μg/kg and about 1,000 μg/kg, between about 650 μg/kg and about 1,000 μg/kg, between about 700 μg/kg and about 1,000 μg/kg, between about 750 μg/kg and about 1,000 μg/kg, between about 800 μg/kg and about 1,000 μg/kg, between about 850 μg/kg and about 1,000 μg/kg, between about 900 μg/kg and about 1,000 μg/kg, or between about 950 μg/kg and about 1,000 μg/kg.

32. The method of any one of claims 1 to 31, wherein the modified IL-7 protein is administered at a dose of between about 700 μg/kg and about 900 μg/kg, between about 750 μg/kg and about 950 μg/kg, between about 700 μg/kg and about 850 μg/kg, between about 750 μg/kg and about 850 μg/kg, between about 700 μg/kg and about 800 μg/kg, between about 800 μg/kg and about 900 μg/kg, between about 750 μg/kg and about 850 μg/kg, or between about 850 μg/kg and about 950 μg/kg.

33. The method of any one of claims 1 to 32, wherein the modified IL-7 protein is administered at a dose of about 650 μg/kg, about 680 μg/kg, about 700 μg/kg, about 720 μg/kg, about 740 μg/kg, about 750 μg/kg, about 760 μg/kg, about 780 μg/kg, about 800 μg/kg, about 820 μg/kg, about 840 μg/kg, about 850 μg/kg, about 860 μg/kg, about 880 μg/kg, about 900 μg/kg, about 920 μg/kg, about 940 μg/kg, about 950 μg/kg, about 960 μg/kg, about 980 μg/kg, about 1000 μg/kg, about 1100 μg/kg, about 1200 μg/kg, about 1,300 μg/kg, about 1,400 μg/kg, about 1,440 μg/kg, about 1,500 μg/kg, about 1,600 μg/kg, about 1,700 μg/kg, about 1,800 μg/kg, about 1,900 μg/kg, or about 2,000 μg/kg.

34. The method of any one of claims 1 to 33, wherein the modified IL-7 protein is administered at a dosing frequency of once a week, once in two weeks, once in three weeks, once in four weeks, once in five weeks, once in six weeks, once in seven weeks, once in eight weeks, once in nine weeks, once in 10 weeks, once in 11 weeks, or once in 12 weeks.

35. The method of any one of claims 1 to 34, wherein the bispecific antibody is administered to the subject at a dose of about 0.1 mg/kg to about 20 mg/kg.

36. The method of any one of claims 1 to 35, wherein the modified IL-7 protein is administered to the subject parenthetically, intramuscularly, subcutaneously, ophthalmic, intravenously, intraperitoneally, intradermally, intraorbitally, intracerebrally, intracranially, intraspinally, intraventricular, intrathecally, intracistemally, intracapsularly, or intratumorally.

37. The method of any one of claims 1 to 36, wherein the bispecific antibody is administered to the subject parenthetically, intramuscularly, subcutaneously, ophthalmic, intravenously, intraperitoneally, intradermally, intraorbitally, intracerebrally, intracranially, intraspinally, intraventricular, intrathecally, intracistemally, intracapsularly, or intratumorally.

38. The method of claim 37, wherein the bispecific antibody is administered intratumorally.

39. The method of any one of claims 1 to 38, further comprising administering at least one additional therapeutic agent to the subject.

40. The method of any one of claims 1 to 39, wherein the tumor is derived from a cancer comprising a breast cancer, head and neck cancer, uterine cancer, brain cancer, skin cancer, renal cancer, lung cancer, colorectal cancer, prostate cancer, liver cancer, bladder cancer, kidney cancer, pancreatic cancer, thyroid cancer, esophageal cancer, eye cancer, stomach (gastric) cancer, gastrointestinal cancer, ovarian cancer, carcinoma, sarcoma, leukemia, lymphoma, myeloma, or a combination thereof.
